(19) United States
(12) Patent Application Publication
(10) Pub. No.: US 2021/0285042 A1
(43) Pub. Date: Sep. 16, 2021

(54) SYSTEMS AND METHODS FOR CALLING VARIANTS USING METHYLATION SEQUENCING DATA

(71) Applicant: GRAIL, Inc., Menlo Park, CA (US)

(72) Inventors: Pranav Singh, Menlo Park, CA (US); Christopher Chang, Mountain View, CA (US); Collin Melton, Menlo Park, CA (US); Oliver Claude Venn, San Francisco, CA (US)

(21) Appl. No.: 17/185,885

(22) Filed: Feb. 25, 2021

Related U.S. Application Data
(60) Provisional application No. 62/983,404, filed on Feb. 28, 2020.

(51) Int. Cl. C12Q 1/6869

(52) U.S. Cl. CPC C12Q 1/6869; C12Q 2600/154

(57) ABSTRACT

An allelic position variant calling method using a prior genotype probability at the allelic position is provided. A strand specific base count set in forward and reverse directions for the allelic position is obtained, using strand orientation and identity of a respective base at the allelic position in each respective nucleic acid fragment sequence that maps to the allelic position, where bases at the allelic position whose identity can be affected by conversion of cytosine to uracil do not contribute to the strand specific base count set. Respective forward and reverse strand conditional probabilities are computed for each candidate genotype for the allelic position using the strand specific base count set and sequencing error estimate. Likelihoods are computed using a combination of these conditional probabilities and the prior genotype probability. From this, a determination is made as to whether the likelihoods support a variant call at the allelic position.

### CROSS REFERENCE TO RELATED PATENT APPLICATION

This application claims priority to U.S. Provisional Patent Application No. 62/983,404, entitled “SYSTEMS AND METHODS FOR CALLING VARIANTS USING METHYLATION SEQUENCING DATA,” filed Feb. 28, 2020 which is hereby incorporated by reference.

### TECHNICAL FIELD

This specification describes using methylation sequencing, in particular, sequencing of nucleic acid samples from biological samples obtained from a subject, to determine genomic variants of a subject.

### BACKGROUND

The increasing knowledge of the molecular basis for cancer and the rapid development of next-generation sequencing techniques are advancing the study of early molecular alterations involved in cancer development in body fluids. Large scale sequencing technologies, such as next-generation sequencing (NGS), have afforded the opportunity to achieve sequencing at costs that are less than one U.S. dollar per million bases, and in fact costs of less than ten U.S. cents per million bases have been realized. Specific genetic and epigenetic alterations associated with such cancer development are found in plasma, serum, and urine cell-free DNA (cfDNA). Such alterations could potentially be used as diagnostic biomarkers for several classes of cancers.

Cell-free DNA (cfDNA) can be found in serum, plasma, urine, and other body fluids representing a “liquid biopsy,” which is a circulating picture of a specific disease. This represents a potential, non-invasive method of screening for a variety of cancers.

cfDNA originates from necrotic or apoptotic cells, and it is generally released by all types of cells. Specific cancer alterations can be found in cfDNA of patients. cfDNA contains specific tumor-related alterations, such as mutations, methylation, and copy number variations (CNVs).

The presence of cfDNA in plasma or serum is well characterized. However, ucfDNA can also be a promising source of biomarkers.

In blood, apoptosis is a frequent event that determines the amount of cfDNA. In cancer patients, however, the amount of cfDNA can also be influenced by necrosis. Since apoptosis seems to be the main release mechanism circulating cfDNA has a size distribution that reveals an enrichment in short fragments of about 167 bp, corresponding to nucleosomes generated by apoptotic cells.

The amount of circulating cfDNA in serum and plasma seems to be significantly higher in patients with tumors than in healthy controls, especially in those with advanced-stage tumors than in early-stage tumors. The variability of the amount of circulating cfDNA is higher in cancer patients than in healthy individuals and the amount of circulating cfDNA is influenced by several physiological and pathological conditions, including proinflammatory diseases.

Methylation status and other epigenetic modifications can be correlated with the presence of some disease conditions such as cancer. And specific patterns of methylation have been determined to be associated with particular cancer conditions. The methylation patterns can be observed even in cell-free DNA.

Given the promise of circulating cfDNA, as well as other forms of genotypic data, as a diagnostic indicator, ways of assessing such data for genomic variant information are needed in the art.

### SUMMARY

The present disclosure addresses the shortcomings identified in the background by providing robust techniques for determining genomic variants from biological samples obtained from a subject using nucleic acid data. The combination of methylation data with whole genome or targeted genome sequencing data provides additional diagnostic power beyond previous screening methods.

Technical solutions (e.g., computing systems, methods, and non-transitory computer-readable storage mediums) for addressing the above-identified problems with analyzing datasets are provided in the present disclosure.

The following presents a summary of the invention in order to provide a basic understanding of some of the aspects of the invention. This summary is not an extensive overview of the invention. It is not intended to identify key/critical elements of the invention or to delineate the scope of the invention. Its sole purpose is to present some of the concepts of the invention in a simplified form as a prelude to the more detailed description that is presented later.

One aspect of the present disclosure provides a method of calling a variant at an allelic position in a test subject. The method comprises, at a computer system having one or more processors, and memory storing one or more programs for execution by the one or more processors, obtaining a prior probability of genotype at the allelic position, for each respective candidate genotype in a set of candidate genotypes, using nucleic acid data acquired from a reference population. The method further comprises obtaining, for the allelic position, a strand-specific base count set. The strand-specific base count set comprises a strand-specific count for each base in a set of bases at the allelic position, in a forward direction and a reverse direction. Each strand-specific base count is acquired by determining (i) a strand orientation and (ii) an identity of a respective base at the allelic position in each respective nucleic acid fragment sequence in a first plurality of nucleic acid fragment sequences, in electronic form, that map to the allelic position, acquired from a first plurality of nucleic acid fragments in a first biological sample of the test subject by methylation sequencing. Bases at the allelic position in the first plurality of nucleic acid fragment sequences whose identity can be affected by conversion of methylated or unmethylated cytosine do not contribute to the strand-specific base count set.

The method further comprises computing a respective forward strand conditional probability and a respective reverse strand conditional probability for each respective candidate genotype in the set of candidate genotypes for the allelic position using the strand-specific base count set and a sequencing error estimate thereby computing a plurality of forward strand conditional probabilities and a plurality of reverse strand conditional probabilities. The method continues by computing a plurality of likelihoods, each respective likelihood in the plurality of likelihoods for a respective candidate genotype in the set of candidate genotypes, using a combination of (i) the respective forward strand conditional probability for the respective candidate genotype in the plurality of forward strand conditional probabilities, (ii) the respective reverse strand conditional probability for the respective candidate genotype in the plurality of reverse strand conditional probabilities, and (iii) the prior probability of genotype for the respective candidate genotype. The method further comprises determining whether the plurality of likelihoods supports a variant call at the allelic position.

In some embodiments, the first biological sample is a liquid biological sample and each respective nucleic acid fragment sequence in the first plurality of nucleic acid fragment sequences represents all or a portion of a respective cell-free nucleic acid molecule in a population of cell-free nucleic acid molecules in the liquid biological sample.

In some embodiments, the first biological sample is a tissue sample and each respective nucleic acid fragment sequence in the first plurality of nucleic acid fragment sequences represents all or a portion of a respective nucleic acid molecule in a population of nucleic acid molecules in the tissue sample. In some embodiments, the tissue sample is a tumor sample from the test subject.

In some embodiments, the reference population comprises at least one hundred reference subjects.

In some embodiments, the first biological sample comprises or consists of blood, whole blood, plasma, serum, urine, cerebrospinal fluid, fecal, saliva, sweat, tears, pleural fluid, pericardial fluid, or peritoneal fluid of the test subject. In some embodiments, the test subject is human.

In some embodiments, the forward direction is a F1R2 read orientation and the reverse direction is a F2R1 read orientation.

In some embodiments, each respective candidate genotype in the set of genotypes is of the form X/Y. In some embodiments, X (e.g., representing maternal allele inheritance) is an identity of the base in the set of bases {A, C, T, G} at the allelic position in a reference genome, and Y (e.g., representing paternal allele inheritance) is an identity of the base in the set of bases {A, C, T, G} at the allelic position in the test subject.

In some embodiments, the set of candidate genotypes consists of between two and ten genotypes in the set {A/A, A/C, A/G, A/T, C/C, C/G, C/T, G/G, G/T, and T/T}. In some embodiments, the set of candidate genotypes comprises at least two genotypes in the set {A/A, A/C, A/G, A/T, C/C, C/G, C/T, G/G, G/T, and T/T}. In some embodiments, the set of candidate genotypes consists of the set {A/A, A/C, A/G, A/T, C/C, C/G, C/T, G/G, G/T, and T/T}.

In some embodiments, a respective likelihood for a respective candidate genotype in the set of candidate genotypes has the form:

Pr(F_{A},F_{G},F_{CT}|F_{ACGT},genotype,ϵ)*Pr(R_{AG},R_{C},R_{T}|R_{ACGT},genotype,ϵ)*Pr(G).

In some such embodiments, Pr(F_{A}, F_{G}, F_{CT}|F_{ACGT}, genotype, ϵ) is the respective forward strand conditional probability for the respective candidate genotype, Pr(R_{AG}, R_{C}, R_{T}|R_{ACGT} genotype, ϵ) is the respective reverse strand conditional probability for the respective candidate genotype, Pr(G) is the prior probability of genotype at the allelic position, acquired by the obtaining step (A) of claim 1, for the respective candidate genotype, ϵ is the sequencing error estimate, genotype is the respective candidate genotype, F_{A} is the forward direction base count for base A at the allelic position across the first plurality of nucleic acid fragment sequences that map to the allelic position from the first biological sample, in the strand specific base count set, F_{G} is the forward direction base count for base G at the allelic position across the first plurality of nucleic acid fragment sequences that map to the allelic position from the first biological sample, in the strand specific base count set, F_{CT} is a summation of (i) the forward direction base count for base C and (ii) the forward direction base count for base T at the allelic position across the first plurality of nucleic acid fragment sequences that map to the allelic position from the first biological sample, in the strand specific base count set, R_{C} is the reverse direction base count for base C at the allelic position across the first plurality of nucleic acid fragment sequences that map to the allelic position from the first biological sample, in the strand specific base count set, R_{T} is the reverse direction base count for base T at the allelic position across the first plurality of nucleic acid fragment sequences that map to the allelic position from the first biological sample, in the strand specific base count set, and R_{AG} is a summation of (i) the reverse direction base count for base A and (ii) the reverse direction base count for base G at the allelic position across the first plurality of nucleic acid fragment sequences that map to the allelic position from the first biological sample, in the strand specific base count set.

In some embodiments, the methylation sequencing is whole-genome methylation sequencing. In some embodiments, the methylation sequencing is targeted DNA methylation sequencing using a plurality of nucleic acid probes. In some embodiments, the plurality of nucleic acid probes comprises one hundred or more probes. In some embodiments, the methylation sequencing detects one or more 5-methylcytosine (5mC) and/or 5-hydroxymethylcytosine (5hmC) in respective nucleic acid fragments in the first plurality of nucleic acid fragments. In some embodiments, the methylation sequencing is bisulfite sequencing where nucleic acid samples are treated with bisulfite to converted unmethylated cytosines to uracils that are subsequently detected as thymines during sequencing analysis. In some embodiments, methylated cytosines undergo enzymatic treatment to be converted to uracils (or a derivative thereof such as dihydrouracils) that are subsequently detected as thymines during sequencing analysis. Unmodified cytosines constitute for about 95% of the total cytosines in the human genome. Conversion of methylated cytosines instead of unmethylated cytosines can lead to fewer alterations to the genome and offer more information for additional analysis such as variant analysis.

In some embodiments, the methylation sequencing comprises conversion of one or more unmethylated cytosines or one or more methylated cytosines, in the nucleic acid fragments in the first plurality of nucleic acid fragments, to a corresponding one or more uracils. In some embodiments, the one or more uracils are detected during the methylation sequencing as one or more corresponding thymines. In some embodiments, the conversion of one or more unmethylated cytosines or one or more methylated cytosines comprises a chemical conversion, an enzymatic conversion, or combinations thereof. In some embodiments, the allelic position is a single base position and the variant is a single nucleotide polymorphism. In some embodiments, the allelic position is a single base position and the variant is a single nucleotide variant.

In some embodiments, the sequencing error estimate is between 0.01 and 0.0001. In some embodiments, the determining whether the plurality of likelihoods support a variant call at the allelic position comprises determining whether the likelihood in the plurality of likelihood corresponding to the reference genotype for the allelic position satisfies a variant threshold, where when the allelic position satisfies a variant threshold, a variant at the allelic position is called. In some embodiments, the reference genotype for the allelic position is A/A, G/G, C/C or T/T.

In some embodiments, the likelihood is expressed as a log-likelihood and the variant threshold is satisfied when the log-likelihood for the reference genotype for the allelic position is less than −10. In some embodiments, the likelihood is expressed as a log-likelihood and the variant threshold is between −25 and −5.

In some embodiments, the method further comprises, when a variant at the allelic position is called, determining an identity of the variant by selecting the candidate genotype in the set of candidate genotypes for the allelic position that has the best likelihood in the plurality of likelihoods as the variant.

In some embodiments, the method further comprises performing the obtaining a respective prior probability of genotype, obtaining a respective strand-specific base count set, computing a respective forward strand conditional probability and a respective reverse strand conditional probability, computing a respective plurality of likelihoods, and determining whether the respective plurality of likelihoods supports a respective variant call for each allelic position in a plurality of allelic positions thereby obtaining a plurality of variant calls for the test subject, where each variant call in the plurality of variant calls is at a different genomic position in a reference genome.

In some embodiments, the method further comprising performing the obtaining a respective prior probability of genotype, obtaining a respective strand-specific base count set, computing a respective forward strand conditional probability and a respective reverse strand conditional probability, computing a respective plurality of likelihoods, and determining whether the respective plurality of likelihoods supports a respective variant call each allelic position in a plurality of allelic positions thereby obtaining a plurality of variant calls for the test subject, where each variant call in the plurality of variant calls is at a different genomic position in a reference genome, and where the first biological sample is a tissue sample, and the methylation sequencing is whole-genome bisulfite sequencing. In some embodiments, the plurality of variant calls comprises 200 variant calls.

In some embodiments, the method further comprises obtaining a second plurality of variant calls using a second plurality of nucleic acid fragment sequences, in electronic form, acquired from a second plurality of nucleic acid fragments in a second biological sample of the test subject by whole genome sequencing, where the second plurality of nucleic acid fragments are cell-free nucleic acid fragments and where the second biological sample is a liquid biological sample, and removing a respective variant call from the plurality of variant calls that is also in the second plurality of variant calls.

In some embodiments, the method further comprises removing a respective variant call from the plurality of variant calls that is in a list of known germline variants. In some embodiments, the method further comprises removing a respective variant call from the plurality of variant calls when the respective variant call is found in a tissue sample of a subject other than the test subject. In some embodiments, the method further comprises removing a respective variant call from the plurality of variant calls when the respective variant call fails to satisfy a quality metric.

In some embodiments, the quality metric is a minimum variant allele fraction in the first plurality of nucleic acid fragment sequences, in electronic form, that map to the allelic position of the respective variant call. In some embodiments, the minimum variant allele fraction is ten percent. In some embodiments, the quality metric is a maximum variant allele fraction in the first plurality of nucleic acid fragment sequences, in electronic form, that map to the allelic position of the respective variant call. In some embodiments, the maximum variant allele fraction is ninety percent. In some embodiments, the quality metric is a minimum depth in the first plurality of nucleic acid fragment sequences, in electronic form, that map to the allelic position of the respective variant call. In some embodiments, the minimum depth is ten.

In some embodiments, the method further comprises using the plurality of variant calls, after the removing, to perform tumor fraction estimation. In some embodiments, the method further comprises using the plurality of variant calls, after the removing, to quantify (e.g., determine or estimate) white blood cell clonal expansion. In some embodiments, the method further comprises using the plurality of variant calls to assess a genetic risk of the subject through germline analysis using the plurality of variant calls.

Another aspect of the present disclosure provides a computing system, comprising one or more processors, and memory storing one or more programs to be executed by the one or more processor. The one or more programs comprise instructions of instructions for calling a variant at an allelic position in a test subject by a method. The method comprises obtaining a prior probability of genotype at the allelic position, for each respective candidate genotype in a set of candidate genotypes, using nucleic acid data acquired from a reference population. The method further comprises obtaining, for the allelic position, a strand-specific base count set, where the strand-specific base count set comprises a strand-specific count for each base in a set of bases {A, C, T, G} at the allelic position, in a forward direction and a reverse direction, that is acquired by determining (i) a strand orientation and (ii) an identity of a respective base at the allelic position in each respective nucleic acid fragment sequence in a first plurality of nucleic acid fragment sequences, in electronic form, that map to the allelic position, acquired from a first plurality of nucleic acid fragments in a first biological sample of the test subject by a methylation sequencing and where bases at the allelic position in the first plurality of nucleic acid fragment sequences whose identity can be affected by conversion of unmethylated cytosine to uracil do not contribute to the strand-specific base count set. The method further comprises computing a respective forward strand conditional probability and a respective reverse strand conditional probability for each respective candidate genotype in the set of candidate genotypes for the allelic position using the strand-specific base count set and a sequencing error estimate thereby computing a plurality of forward strand conditional probabilities and a plurality of reverse strand conditional probabilities. The method further comprises computing a plurality of likelihoods, each respective likelihood in the plurality of likelihoods for a respective candidate genotype in the set of candidate genotypes, using a combination of (i) the respective forward strand conditional probability for the respective candidate genotype in the plurality of forward strand conditional probabilities, (ii) the respective reverse strand conditional probability for the respective candidate genotype in the plurality of reverse strand conditional probabilities, and (iii) the prior probability of genotype for the respective candidate genotype. The method further comprises determining whether the plurality of likelihoods supports a variant call at the allelic position. Another aspect of the present disclosure provides a computing system including the above disclosed one or more programs that further comprise instructions for performing any of the above-disclosed methods alone or in combination.

Another aspect of the present disclosure provides a non-transitory computer-readable storage medium storing one or more programs for calling a variant at an allelic position in a test subject. The one or more programs are configured for execution by a computer. Moreover, the one or more programs comprise instructions for obtaining a prior probability of genotype at the allelic position, for each respective candidate genotype in a set of candidate genotypes, using nucleic acid data acquired from a reference population. The one or more programs further comprise instructions for obtaining, for the allelic position, a strand-specific base count set, where the strand-specific base count set comprises a strand-specific count for each base in a set of bases {A, C, T, G} at the allelic position, in a forward direction and a reverse direction, that is acquired by determining (i) a strand orientation and (ii) an identity of a respective base at the allelic position in each respective nucleic acid fragment sequence in a first plurality of nucleic acid fragment sequences, in electronic form, that map to the allelic position, acquired from a first plurality of nucleic acid fragments in a first biological sample of the test subject by a methylation sequencing and where bases at the allelic position in the first plurality of nucleic acid fragment sequences whose identity can be affected by conversion of unmethylated cytosine to uracil do not contribute to the strand-specific base count set. The one or more programs further comprise instructions for computing a respective forward strand conditional probability and a respective reverse strand conditional probability for each respective candidate genotype in the set of candidate genotypes for the allelic position using the strand-specific base count set and a sequencing error estimate thereby computing a plurality of forward strand conditional probabilities and a plurality of reverse strand conditional probabilities. The one or more programs further comprise instructions for computing a plurality of likelihoods, each respective likelihood in the plurality of likelihoods for a respective candidate genotype in the set of candidate genotypes, using a combination of (i) the respective forward strand conditional probability for the respective candidate genotype in the plurality of forward strand conditional probabilities, (ii) the respective reverse strand conditional probability for the respective candidate genotype in the plurality of reverse strand conditional probabilities, and (iii) the prior probability of genotype for the respective candidate genotype. The one or more programs further comprise instructions for determining whether the plurality of likelihoods support a variant call at the allelic position.

Another aspect of the present disclosure provides non-transitory computer-readable storage medium comprising the above-disclosed one or more programs in which the one or more programs further comprise instructions for performing any of the above-disclosed methods alone or in combination. The one or more programs are configured for execution by a computer.

Still another aspect of the present disclosure provides a computing system comprising one or more processors and memory storing one or more programs to be executed by the one or more processor, the one or more programs comprising instructions performing any of the methods disclosed above.

Various embodiments of systems, methods, and devices within the scope of the appended claims each have several aspects, no single one of which is solely responsible for the desirable attributes described herein. Without limiting the scope of the appended claims, some prominent features are described herein. After considering this discussion, and particularly after reading the section entitled “Detailed Description” one will understand how the features of various embodiments are used.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIG. 1 illustrates an example Venn diagram of subject variants in chromosome 1, in accordance with the prior art, in which a set of variants 20 is identified through whole-genome bisulfite sequencing and an additional set of variants 10 is identified using freebayes reference (Zook et al. 2014, “Integrating human sequence data sets provides a resource of benchmark SNP and indel genotype calls” Nat. Biotech. 32, 246-251). Of the set of somatic variants in the example, three-quarters are not included or identified by current methods.

FIG. 2 illustrates an example block diagram illustrating a computing device in accordance with some embodiments of the present disclosure.

FIGS. 3A, 3B, 3C, and 3D collectively illustrate an example flowchart of a method of calling a variant allele in which dashed boxes represent optional steps in accordance with some embodiments of the present disclosure.

FIG. 4 illustrates an example of germline variants identified from bisulfite-treated biological samples from subjects, in accordance with some embodiments of the present disclosure.

FIG. 5 illustrates an example of somatic variants identified from bisulfite-treated biological samples from subjects, with single strand support for each variant, in accordance with some embodiments of the present disclosure.

FIG. 6 illustrates an example of somatic variants identified from paired whole-genome bisulfite sequencing (WGBS) and whole-genome sequencing (WGS) cell-free nucleic acid fragments, in accordance with some embodiments of the present disclosure.

FIG. 7 illustrates a flowchart of a method for preparing a nucleic acid sample for sequencing in accordance with some embodiments of the present disclosure.

FIG. 8 is a graphical representation of the process for obtaining sequence reads in accordance with some embodiments of the present disclosure

FIG. 9 illustrates an example flowchart of a method for obtaining methylation information for the purposes of screening for a cancer condition in a test subject in accordance with some embodiments of the present disclosure

FIG. 10 illustrates an example calculation of candidate genotype log-likelihoods, in accordance with some embodiments of the present disclosure.

FIG. 11 illustrates an example of blacklisting a portion of a genome for analysis of tissue fraction, in accordance with some embodiments of the present disclosure.

FIG. 12 illustrates an example of filtering variants on the bases of likelihood thresholds, in accordance with some embodiments of the present disclosure.

FIGS. 13A and 13B illustrate two examples of tumor fraction estimation (e.g., 1300 and 1302) that can be performed in accordance with some embodiments of the present disclosure.

FIG. 14 illustrate an example of processing samples for tumor fraction estimation, in accordance with the method of FIG. 13B.

FIG. 15 illustrate performance of the method of FIG. 13B, as further illustrated in FIG. 14, at each stage in a series of filtering steps in accordance with an embodiment of the present disclosure.

FIG. 16 show the sensitivity, specificity, true positive rate, and false positive rate for calling alleles using threshold values of 0, −10, −20, −30, −40, −50, −60, −70, −80 and −90 with paired whole genome bisulfite sequencing (WGBS)/whole genome sequencing (WGS) sequencing data in accordance with an embodiment of the present disclosure.

FIGS. 17A and 17B illustrate two different python scripts for computing tumor fraction in accordance with embodiments of the present disclosure.

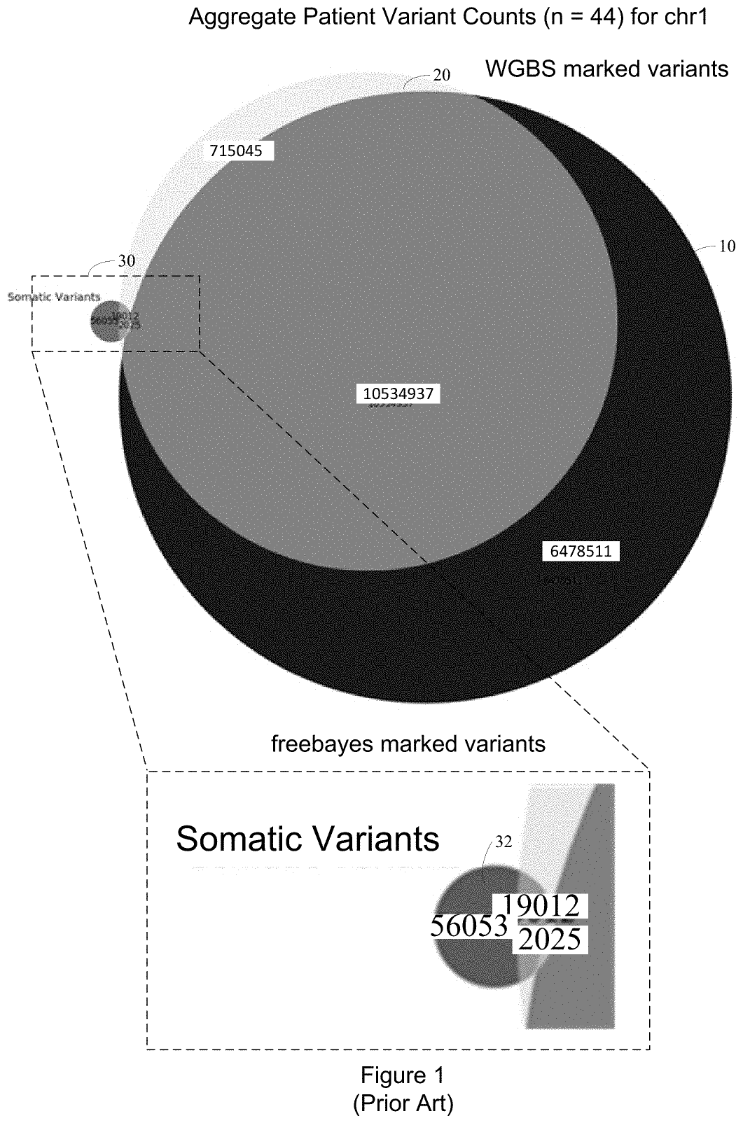

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

The implementations described herein provide various technical solutions for determining variant call at an allelic position for a subject. Prior genotype probabilities are obtained for each respective candidate genotype in a set of candidate genotypes for an allelic position. For the subject, a strand-specific base count set is obtained in a forward and reverse direction for the allelic position. The forward and reverse strand-specific base counts are determined using strand orientation information and identity of a respective base at the allelic position in each respective nucleic acid fragment sequence that maps to the allelic position. Bases at the allelic position whose identity can be affected by conversion of methylated or unmethylated cytosine to uracil do not contribute to the strand-specific base count set. Respective forward and reverse strand conditional probabilities are computed, based on the strand-specific base count set for the subject and an error estimate, for each respective candidate genotype in the set of candidate genotypes. A plurality of candidate genotype likelihoods are computed, each respective likelihood in the plurality of likelihoods for a respective candidate genotype in the set of candidate genotypes. Each likelihood is calculated using a combination of (i) the respective forward strand conditional probability for the respective candidate genotype in the plurality of forward strand conditional probabilities, (ii) the respective reverse strand conditional probability for the respective candidate genotype in the plurality of reverse strand conditional probabilities, and (iii) the prior probability of genotype for the respective candidate genotype. A determination is made whether the plurality of likelihoods supports a variant call at the allelic position for the subject.

### Definitions

As used herein, the term “about” or “approximately” means within an acceptable error range for the particular value as determined by one of ordinary skill in the art, which depends in part on how the value is measured or determined, e.g., the limitations of the measurement system. For example, in some embodiments “about” mean within 1 or more than 1 standard deviation, per the practice in the art. In some embodiments, “about” means a range of ±20%, ±10%, ±5%, or ±1% of a given value. In some embodiments, the term “about” or “approximately” means within an order of magnitude, within 5-fold, or within 2-fold, of a value. Where particular values are described in the application and claims, unless otherwise stated the term “about” meaning within an acceptable error range for the particular value can be assumed. The term “about” can have the meaning as commonly understood by one of ordinary skill in the art. In some embodiments, the term “about” refers to ±10%. In some embodiments, the term “about” refers to ±5%.

As used herein, the term “assay” refers to a technique for determining a property of a substance, e.g., a nucleic acid, a protein, a cell, a tissue, or an organ. An assay (e.g., a first assay or a second assay) can comprise a technique for determining the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay can be used to detect any of the properties of nucleic acids mentioned herein. Properties of a nucleic acids can include a sequence, genomic identity, copy number, methylation state at one or more nucleotide positions, size of the nucleic acid, presence or absence of a mutation in the nucleic acid at one or more nucleotide positions, and pattern of fragmentation of a nucleic acid (e.g., the nucleotide position(s) at which a nucleic acid fragments). An assay or method can have a particular sensitivity and/or specificity, and their relative usefulness as a diagnostic tool can be measured using ROC-AUC statistics.

As disclosed herein, the term “biological sample” refers to any sample taken from a subject, which can reflect a biological state associated with the subject, and that includes cell-free DNA. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. A biological sample can include any tissue or material derived from a living or dead subject. A biological sample can be a cell-free sample. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis.

As disclosed herein, the terms “nucleic acid” and “nucleic acid molecule” are used interchangeably. The terms refer to nucleic acids of any composition form, such as deoxyribonucleic acid (DNA, e.g., complementary DNA (cDNA), genomic DNA (gDNA) and the like), ribonucleic acid (RNA, e.g., message RNA (mRNA), short inhibitory RNA (siRNA), ribosomal RNA (rRNA), transfer RNA (tRNA), microRNA, RNA highly expressed by the fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid in some embodiments can be from a single chromosome or fragment thereof (e.g., a nucleic acid sample may be from one chromosome of a sample obtained from a diploid organism). In certain embodiments, nucleic acids comprise nucleosomes, fragments or parts of nucleosomes or nucleosome-like structures. Nucleic acids sometimes comprise protein (e.g., histones, DNA binding proteins, and the like). Nucleic acids analyzed by processes described herein sometimes are substantially isolated and are not substantially associated with protein or other molecules. Nucleic acids also include derivatives, variants and analogs of RNA or DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides include deoxyadenosine, deoxycytidine, deoxyguanosine, and deoxythymidine. For RNA, the base cytosine is replaced with uracil and the sugar 2′ position includes a hydroxyl moiety. A nucleic acid may be prepared using a nucleic acid obtained from a subject as a template.

As disclosed herein, the terms “cell-free nucleic acid,” “cell-free DNA,” and “cfDNA” interchangeably refer to nucleic acid fragments that circulate in a subject's body (e.g., in a bodily fluid such as the bloodstream) and originate from one or more healthy cells and/or from one or more cancer cells. Cell-free DNA may be recovered from bodily fluids such as blood, whole blood, plasma, serum, urine, cerebrospinal fluid, fecal, saliva, sweat, sweat, tears, pleural fluid, pericardial fluid, or peritoneal fluid of a subject. Cell-free nucleic acids are used interchangeably with circulating nucleic acids. Examples of the cell-free nucleic acids include but are not limited to RNA, mitochondrial DNA, or genomic DNA.

As disclosed herein, the term “circulating tumor DNA” or “ctDNA” refers to nucleic acid fragments that originate from aberrant tissue, such as the cells of a tumor or other types of cancer, which may be released into a subject's bloodstream as result of biological processes such as apoptosis or necrosis of dying cells or actively released by viable tumor cells.

As disclosed herein, the term “reference genome” refers to any particular known, sequenced or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As disclosed herein, the term “regions of a reference genome,” “genomic region,” or “chromosomal region” refers to any portion of a reference genome, contiguous or non-contiguous. It can also be referred to, for example, as a bin, a partition, a genomic portion, a portion of a reference genome, a portion of a chromosome and the like. In some embodiments, a genomic section is based on a particular length of the genomic sequence. In some embodiments, a method can include analysis of multiple mapped sequence reads to a plurality of genomic regions. Genomic regions can be approximately the same length or the genomic sections can be different lengths. In some embodiments, genomic regions are of about equal length. In some embodiments, genomic regions of different lengths are adjusted or weighted. In some embodiments, a genomic region is about 10 kilobases (kb) to about 500 kb, about 20 kb to about 400 kb, about 30 kb to about 300 kb, about 40 kb to about 200 kb, and sometimes about 50 kb to about 100 kb. In some embodiments, a genomic region is about 100 kb to about 200 kb. A genomic region is not limited to contiguous runs of sequence. Thus, genomic regions can be made up of contiguous and/or non-contiguous sequences. A genomic region is not limited to a single chromosome. In some embodiments, a genomic region includes all or part of one chromosome or all or part of two or more chromosomes. In some embodiments, genomic regions may span one, two, or more entire chromosomes. In addition, the genomic regions may span joint or disjointed portions of multiple chromosomes.

As used herein, the term “nucleic acid fragment sequence” refers to all or a portion of a polynucleotide sequence of at least three consecutive nucleotides. In the context of sequencing nucleic acid fragments found in a biological sample, the term “nucleic acid fragment sequence” refers to the sequence of a nucleic acid molecule (e.g., a DNA fragment) that is found in the biological sample or a representation thereof (e.g., an electronic representation of the sequence). Sequencing data (e.g., raw or corrected sequence reads from whole-genome sequencing, targeted sequencing, etc.) from a unique nucleic acid fragment (e.g., a cell-free nucleic acid) are used to determine a nucleic acid fragment sequence. Such sequence reads, which in fact may be obtained from sequencing of PCR duplicates of the original nucleic acid fragment, therefore “represent” or “support” the nucleic acid fragment sequence. There may be a plurality of sequence reads that each represents or supports a particular nucleic acid fragment in a biological sample (e.g., PCR duplicates), however, there may be one nucleic acid fragment sequence for the particular nucleic acid fragment. In some embodiments, duplicate sequence reads generated for the original nucleic acid fragment are combined or removed (e.g., collapsed into a single sequence, e.g., the nucleic acid fragment sequence). Accordingly, when determining metrics relating to a population of nucleic acid fragments, in a sample, that each encompass a particular locus (e.g., an abundance value for the locus or a metric based on a characteristic of the distribution of the fragment lengths), the nucleic acid fragment sequences for the population of nucleic acid fragments, rather than the supporting sequence reads (e.g., which may be generated from PCR duplicates of the nucleic acid fragments in the population, can be used to determine the metric. This is because, in such embodiments, one copy of the sequence is used to represent the original (e.g., unique) nucleic acid fragment (e.g., unique nucleic acid molecule). It is noted that the nucleic acid fragment sequences for a population of nucleic acid fragments may include several identical sequences, each of which represents a different original nucleic acid fragment, rather than duplicates of the same original nucleic acid fragment. In some embodiments, a cell-free nucleic acid is considered a nucleic acid fragment.

The terms “sequence reads” or “reads,” used interchangeably herein, refer to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130 bp, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp.

As disclosed herein, the terms “sequencing,” “sequence determination,” and the like as used herein refers generally to any and all biochemical processes that may be used to determine the order of biological macromolecules such as nucleic acids or proteins. For example, sequencing data can include all or a portion of the nucleotide bases in a nucleic acid molecule such as a DNA fragment.

As disclosed herein, the term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.”

As used herein, the term “methylation” refers to a modification of deoxyribonucleic acid (DNA) where a hydrogen atom on the pyrimidine ring of a cytosine base is converted to a methyl group, forming 5-methylcytosine. In particular, methylation tends to occur at dinucleotides of cytosine and guanine referred to herein as “CpG sites”. In other instances, methylation may occur at a cytosine not part of a CpG site or at another nucleotide that's not cytosine; however, these are rarer occurrences. In this present disclosure, methylation is discussed in reference to CpG sites for the sake of clarity. Anomalous cfDNA methylation can be identified as hypermethylation or hypomethylation, both of which may be indicative of cancer status. As is well known in the art, DNA methylation anomalies (compared to healthy controls) can cause different effects, which may contribute to cancer.

Various challenges arise in the identification of anomalously methylated cfDNA fragments. First, determining a subject's cfDNA to be anomalously methylated only holds weight in comparison with a group of control subjects, such that if the control group is small in number, the determination loses confidence with the small control group. Additionally, among a group of control subjects' methylation status can vary which can be difficult to account for when determining a subject's cfDNA to be anomalously methylated. On another note, methylation of a cytosine at a CpG site causally influences methylation at a subsequent CpG site.

The principles described herein are equally applicable for the detection of methylation in a non-CpG context, including non-cytosine methylation. Further, the methylation state vectors may contain elements that are generally vectors of sites where methylation has or has not occurred (even if those sites are not CpG sites specifically). With that substitution, the remainder of the processes described herein are the same, and consequently, the inventive concepts described herein are applicable to those other forms of methylation.

As used herein the term “methylation index” for each genomic site (e.g., a CpG site, a region of DNA where a cytosine nucleotide is followed by a guanine nucleotide in the linear sequence of bases along its 5′→3′ direction) can refer to the proportion of sequence reads showing methylation at the site over the total number of reads covering that site. The “methylation density” of a region can be the number of reads at sites within a region showing methylation divided by the total number of reads covering the sites in the region. The sites can have specific characteristics, (e.g., the sites can be CpG sites). The “CpG methylation density” of a region can be the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of unconverted cytosines (which can correspond to methylated cytosine) at CpG sites as a proportion of all CpG sites covered by sequence reads mapped to the 100-kb region. In some embodiments, this analysis is performed for other bin sizes, e.g., 50-kb or 1-Mb, etc. In some embodiments, a region is an entire genome or a chromosome or part of a chromosome (e.g., a chromosomal arm). A methylation index of a CpG site can be the same as the methylation density for a region when the region includes that CpG site. The “proportion of methylated cytosines” can refer the number of cytosine sites, “C's,” that are shown to be methylated (for example unconverted after bisulfite conversion) over the total number of analyzed cytosine residues, e.g., including cytosines outside of the CpG context, in the region. The methylation index, methylation density and proportion of methylated cytosines are examples of “methylation levels.”

As used herein, the term “methylation profile” (also called methylation status) can include information related to DNA methylation for a region. Information related to DNA methylation can include a methylation index of a CpG site, a methylation density of CpG sites in a region, a distribution of CpG sites over a contiguous region, a pattern or level of methylation for each individual CpG site within a region that contains more than one CpG site, and non-CpG methylation. A methylation profile of a substantial part of the genome can be considered equivalent to the methylome. “DNA methylation” in mammalian genomes can refer to the addition of a methyl group to position 5 of the heterocyclic ring of cytosine (e.g., to produce 5-methylcytosine) among CpG dinucleotides. Methylation of cytosine can occur in cytosines in other sequence contexts, for example, 5′-CHG-3′ and 5′-CHH-3′, where H is adenine, cytosine or thymine. Cytosine methylation can also be in the form of 5-hydroxymethylcytosine. Methylation of DNA can include methylation of non-cytosine nucleotides, such as N6-methyladenine.

As disclosed herein, the term “subject,” “reference subject,” or “test subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale, and shark. The terms “subject” and “patient” are used interchangeably herein and refer to a human or non-human animal who is known to have, or potentially has, a medical condition or disorder, such as, e.g., a cancer. In some embodiments, a subject is a male or female of any stage (e.g., a man, a woman, or a child).

A subject from whom a sample is taken, or is treated by any of the methods or compositions described herein can be of any age and can be an adult, infant or child. In some cases, the subject, e.g., patient is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 years old, or within a range therein (e.g., between about 2 and about 20 years old, between about 20 and about 40 years old, or between about 40 and about 90 years old). A particular class of subjects, e.g., patients that can benefit from a method of the present disclosure is subjects, e.g., patients over the age of 40.

Another particular class of subjects, e.g., patients that can benefit from a method of the present disclosure is pediatric patients, who can be at higher risk of chronic heart symptoms. Furthermore, a subject, e.g., a patient from whom a sample is taken, or is treated by any of the methods or compositions described herein, can be male or female.

The term “normalize” as used herein means transforming a value or a set of values to a common frame of reference for comparison purposes. For example, when a diagnostic ctDNA level is “normalized” with a baseline ctDNA level, the diagnostic ctDNA level is compared to the baseline ctDNA level so that the amount by which the diagnostic ctDNA level differs from the baseline ctDNA level can be determined.

As used herein the term “cancer” or “tumor” refers to an abnormal mass of tissue in which the growth of the mass surpasses and is not coordinated with the growth of normal tissue. A cancer or tumor can be defined as “benign” or “malignant” depending on the following characteristics: a degree of cellular differentiation including morphology and functionality, rate of growth, local invasion and metastasis. A “benign” tumor can be well-differentiated, have characteristically slower growth than a malignant tumor and remain localized to the site of origin. In addition, in some cases a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites.

As used herein, the term “tissue” corresponds to a group of cells that group together as a functional unit. More than one type of cell can be found in a single tissue. Different types of tissue may consist of different types of cells (e.g., hepatocytes, alveolar cells or blood cells), but also can correspond to tissue from different organisms (mother vs. fetus) or to healthy cells vs. tumor cells. The term “tissue” can generally refer to any group of cells found in the human body (e.g., heart tissue, lung tissue, kidney tissue, nasopharyngeal tissue, oropharyngeal tissue). In some aspects, the term “tissue” or “tissue type” can be used to refer to a tissue from which a cell-free nucleic acid originates. In one example, viral nucleic acid fragments can be derived from blood tissue. In another example, viral nucleic acid fragments can be derived from tumor tissue.

As used herein the term “untrained classifier” refers to a classifier that has not been trained on a target dataset. For instance, consider the case of a first canonical set of methylation state vectors and a second canonical set of methylation state vectors discussed below. The respective canonical sets of methylation state vectors are applied as collective input to an untrained classifier, in conjunction with the cell source of each respective reference subject represented by the first canonical set of methylation state vectors (hereinafter “primary training dataset”) to train the untrained classifier on cell source thereby obtaining a trained classifier. Moreover, it will be appreciated that the term “untrained classifier” does not exclude the possibility that transfer learning techniques are used in such training of the untrained classifier. For instance, Fernandes et al., 2017, “Transfer Learning with Partial Observability Applied to Cervical Cancer Screening,” Pattern Recognition and Image Analysis: 8^{th} Iberian Conference Proceedings, 243-250, which is hereby incorporated by reference, provides non-limiting examples of such transfer learning. In instances where transfer learning is used, the untrained classifier described above is provided with additional data over and beyond that of the primary training dataset. That is, in non-limiting examples of transfer learning embodiments, the untrained classifier receives (i) canonical sets of methylation state vectors and the cell source labels of each of the reference subjects represented by canonical sets of methylation state vectors (“primary training dataset”) and (ii) additional data. Typically, this additional data is in the form of coefficients (e.g., regression coefficients) that were learned from another, auxiliary training dataset. Moreover, while a description of a single auxiliary training dataset has been disclosed, it will be appreciated that there is no limit on the number of auxiliary training datasets that may be used to complement the primary training dataset in training the untrained classifier in the present disclosure. For instance, in some embodiments, two or more auxiliary training datasets, three or more auxiliary training datasets, four or more auxiliary training datasets or five or more auxiliary training datasets are used to complement the primary training dataset through transfer learning, where each such auxiliary dataset is different than the primary training dataset. Any manner of transfer learning may be used in such embodiments. For instance, consider the case where there is a first auxiliary training dataset and a second auxiliary training dataset in addition to the primary training dataset. The coefficients learned from the first auxiliary training dataset (by application of a classifier such as regression to the first auxiliary training dataset) may be applied to the second auxiliary training dataset using transfer learning techniques (e.g., the above described two-dimensional matrix multiplication), which in turn may result in a trained intermediate classifier whose coefficients are then applied to the primary training dataset and this, in conjunction with the primary training dataset itself, is applied to the untrained classifier. Alternatively, a first set of coefficients learned from the first auxiliary training dataset (by application of a classifier such as regression to the first auxiliary training dataset) and a second set of coefficients learned from the second auxiliary training dataset (by application of a classifier such as regression to the second auxiliary training dataset) may each individually be applied to a separate instance of the primary training dataset (e.g., by separate independent matrix multiplications) and both such applications of the coefficients to separate instances of the primary training dataset in conjunction with the primary training dataset itself (or some reduced form of the primary training dataset such as principal components or regression coefficients learned from the primary training set) may then be applied to the untrained classifier in order to train the untrained classifier. In either example, knowledge regarding cell source (e.g., cancer type, etc.) derived from the first and second auxiliary training datasets is used, in conjunction with the cell source labeled primary training dataset), to train the untrained classifier.

The term “classification” can refer to any number(s) or other characters(s) that are associated with a particular property of a sample. For example, a “+” symbol (or the word “positive”) can signify that a sample is classified as having deletions or amplifications. In another example, the term “classification” refers to an amount of tumor tissue in the subject and/or sample, a size of the tumor in the subject and/or sample, a stage of the tumor in the subject, a tumor load in the subject and/or sample, and presence of tumor metastasis in the subject. In some embodiments, the classification is binary (e.g., positive or negative) or has more levels of classification (e.g., a scale from 1 to 10 or 0 to 1). In some embodiments, the terms “cutoff” and “threshold” refer to predetermined numbers used in an operation. In one example, a cutoff size refers to a size above which fragments are excluded. In some embodiments, a threshold value is a value above or below which a particular classification applies. Either of these terms can be used in either of these contexts.

As used herein, the terms “control,” “control sample,” “reference,” “reference sample,” “normal,” and “normal sample” describe a sample from a subject that does not have a particular condition, or is otherwise healthy. In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map sequence reads obtained from sequencing a sample from the subject. A reference genome can refer to a haploid or diploid genome to which sequence reads from the biological sample and a constitutional sample can be aligned and compared. An example of a constitutional sample can be DNA of white blood cells obtained from the subject. For a haploid genome, there can be only one nucleotide at each locus. For a diploid genome, heterozygous loci can be identified; each heterozygous locus can have two alleles, where either allele can allow a match for alignment to the locus.

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

### Exemplary System Embodiments

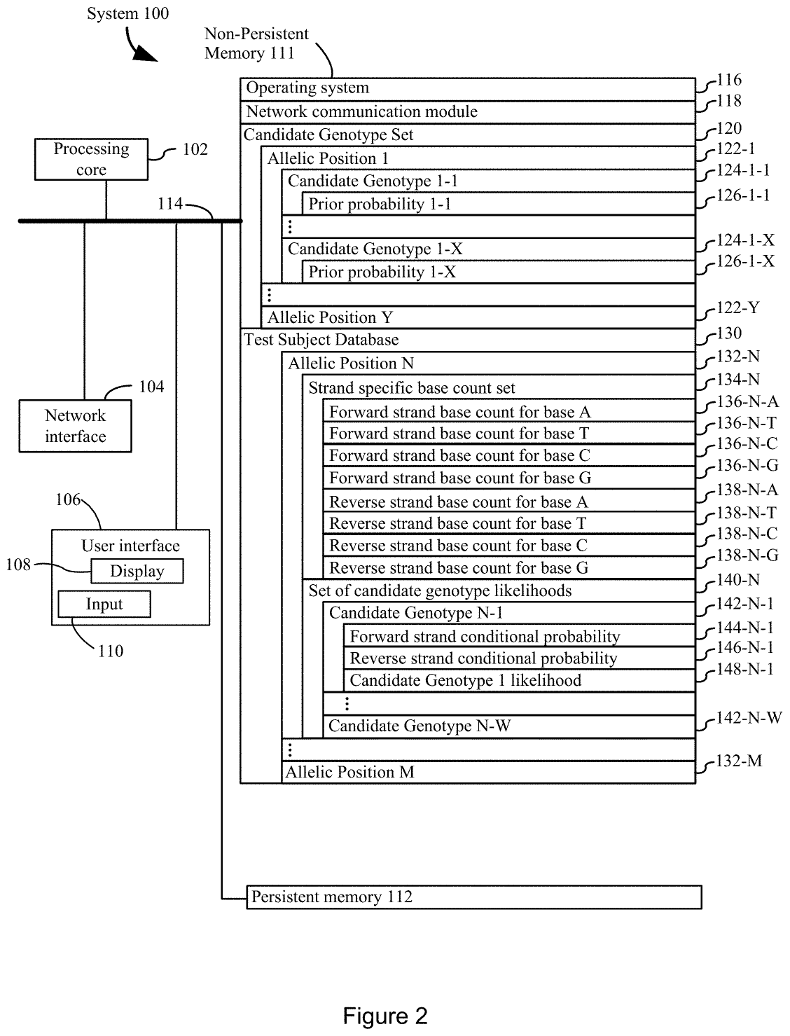

Details of an exemplary system are now described in conjunction with FIG. 2. FIG. 2 is a block diagram illustrating system 100 in accordance with some implementations. Device 100 in some implementations includes one or more processing units CPU(s) 102 (also referred to as processors or processing core), one or more network interfaces 104, user interface 106, non-persistent memory 111, persistent memory 112, and one or more communication buses 114 for interconnecting these components. One or more communication buses 114 optionally include circuitry (sometimes called a chipset) that interconnects and controls communications between system components. Non-persistent memory 111 typically includes high-speed random access memory, such as DRAM, SRAM, DDR RAM, ROM, EEPROM, flash memory, whereas persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid-state storage devices. Persistent memory 112 optionally includes one or more storage devices remotely located from the CPU(s) 102. Persistent memory 112, and the non-volatile memory device(s) within non-persistent memory 112, comprise non-transitory computer-readable storage medium. In some implementations, non-persistent memory 111 or alternatively non-transitory computer-readable storage medium stores the following programs, modules and data structures, or a subset thereof, sometimes in conjunction with persistent memory 112:
optional instructions, programs, data, or information associated with optional operating system 116, which includes procedures for handling various basic system services and for performing hardware dependent tasks;
instructions, programs, data, or information associated with an optional network communication module (or instructions) 118 for connecting the system 100 with other devices, or a communication network;
instructions, programs, data, or information associated with a candidate genotype set 120 that stores, for each allelic position 122 in a reference genome for a species, a respective candidate genotype 124 and a corresponding prior probability 126 of said candidate genotype, where the prior probabilities are based on nucleic acid sequence data collected from a population of reference subjects of the species; and
a test subject database including, for at least one allelic position 132-N, a strand-specific base count set 134-N and a set of candidate genotype probabilities 140-N, where the strand specific base count set 134-N comprises a respective forward strand base count 136 and a respective reverse strand base count 138 for each base in the set of {A, T, C, G}, and the set of candidate genotype probabilities 140 comprises, for each candidate genotype 142-N of the allelic position 132-N, a respective forward strand conditional probability 144, a respective reverse strand conditional probability 146, and a candidate genotype likelihood 148.

In some implementations, one or more of the above-identified elements are stored in one or more of the previously mentioned memory devices, and correspond to a set of instructions for performing a function described above. The above-identified modules, data, or programs (e.g., sets of instructions) may not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above-identified elements is stored in a computer system, other than that of visualization system 100, that is addressable by visualization system 100 so that visualization system 100 may retrieve all or a portion of such data.

Although FIG. 2 depicts a “system 100,” the figure is intended more as functional description of the various features which may be present in computer systems than as a structural schematic of the implementations described herein. In practice, items shown separately could be combined and some items can be separated. Moreover, although FIG. 2 depicts certain data and modules in non-persistent memory 111, some or all of these data and modules may be in persistent memory 112.

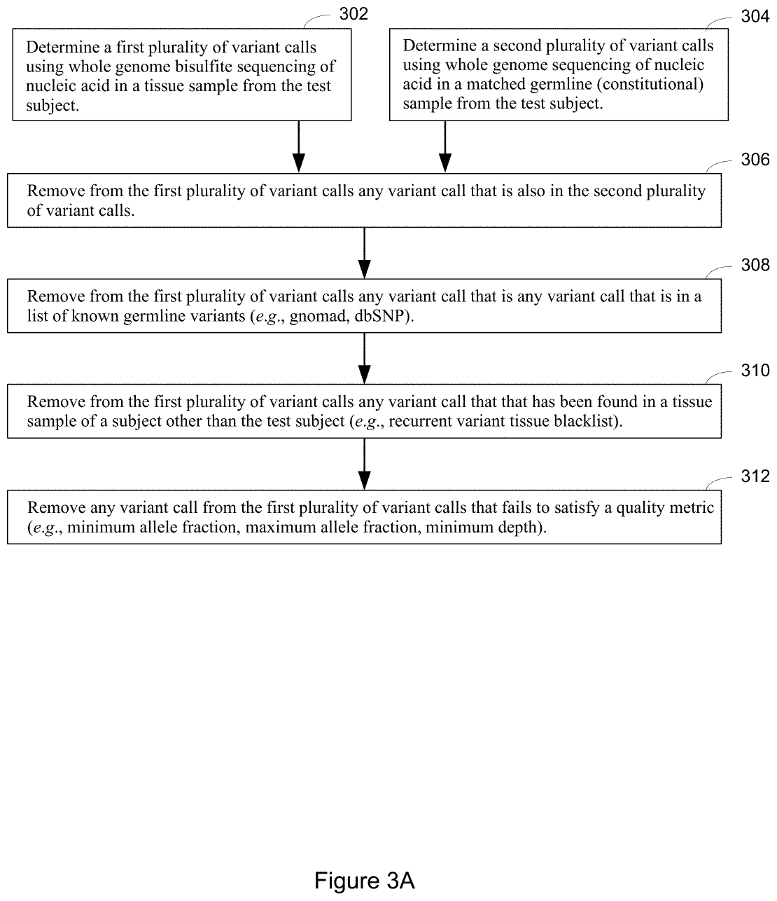

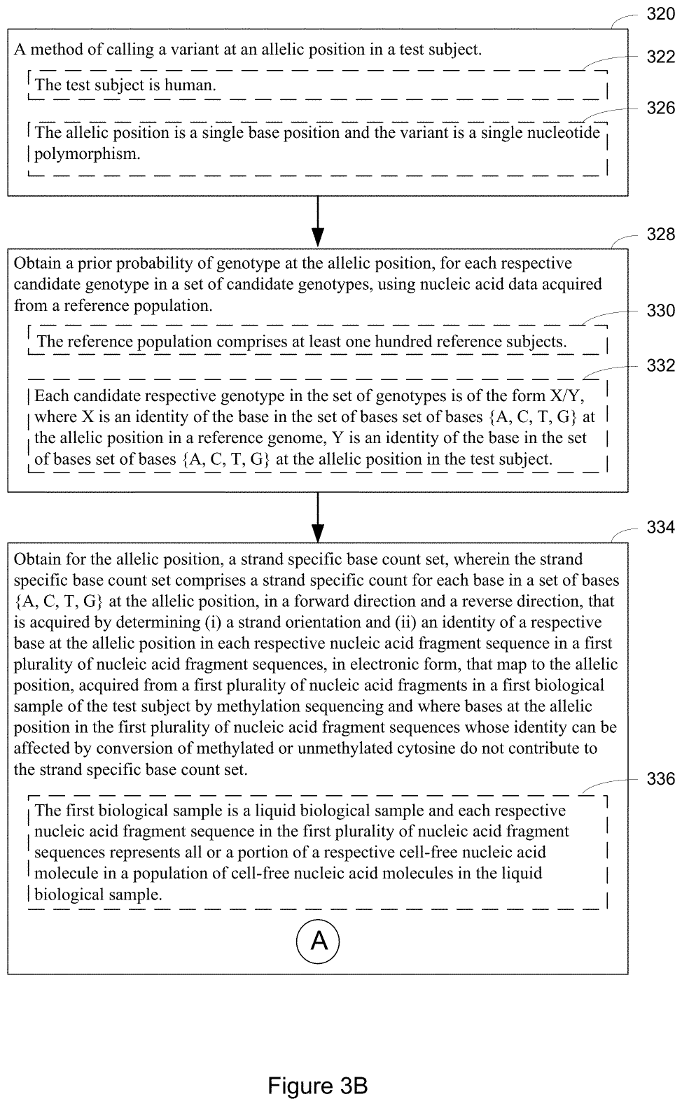

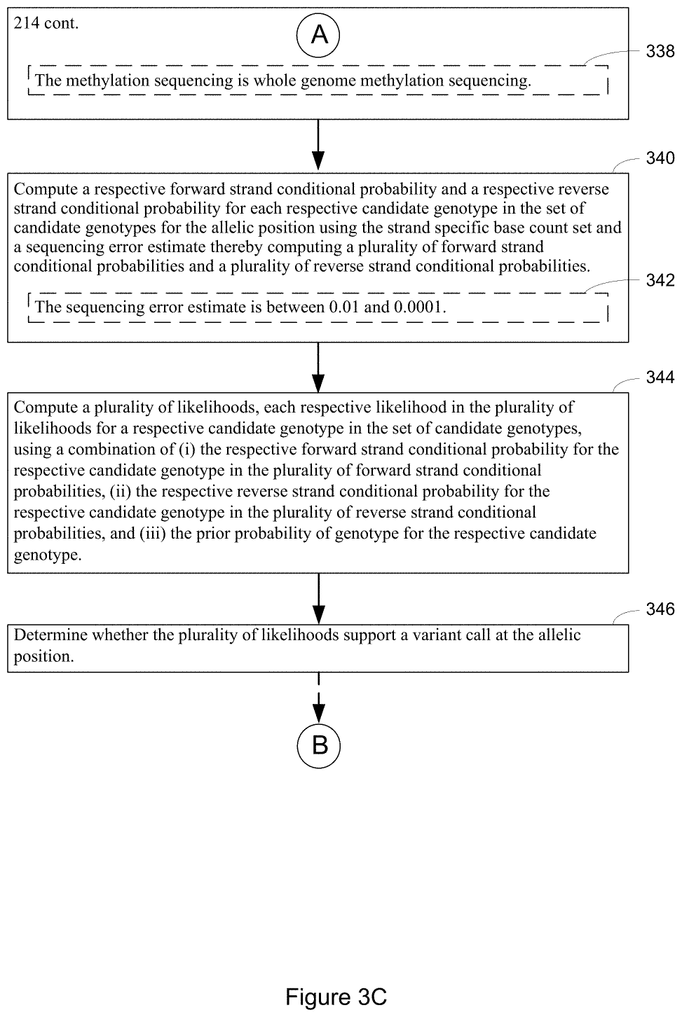

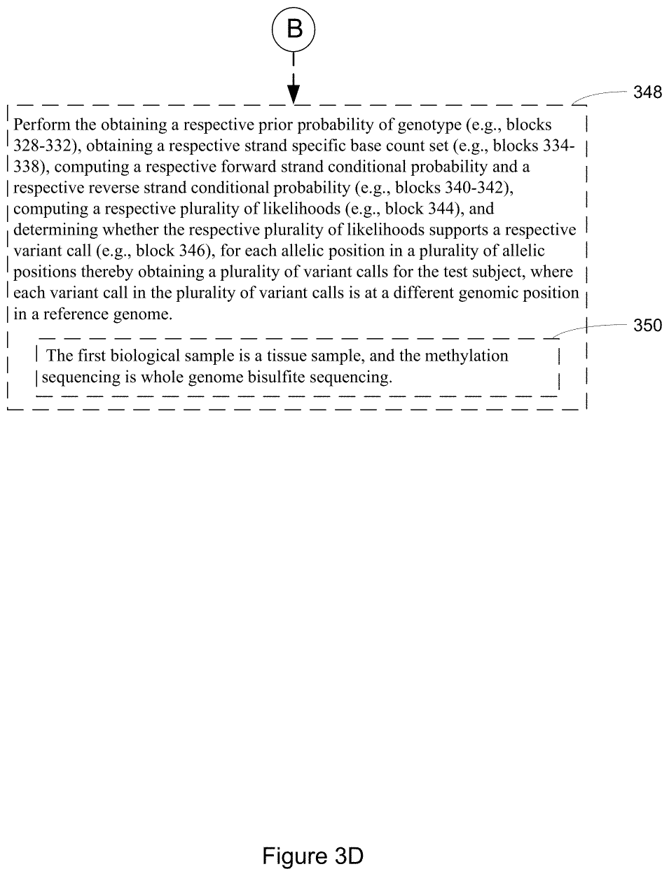

While a system in accordance with the present disclosure has been disclosed with reference to FIG. 2, methods in accordance with the present disclosure are now detailed with reference to FIGS. 3A-3D. Any of the disclosed methods can make use of any of the assays or algorithms disclosed in U.S. patent application Ser. No. 15/793,830, filed Oct. 25, 2017, and/or International Patent Publication No. WO 2018/081130, entitled “Methods and Systems for Tumor Detection,” each of which is hereby incorporated by reference, in order to determine a cancer condition in a test subject or a likelihood that the subject has the cancer condition. For instance, any of the disclosed methods can work in conjunction with any of the disclosed methods or algorithms disclosed in U.S. patent application Ser. No. 15/793,830, filed Oct. 25, 2017, and/or International Patent Publication No. WO 2018/081130, entitled “Methods and Systems for Tumor Detection.”

Identifying Somatic Variants.

FIG. 3A provides an overview of a method of identifying somatic variants in a test subject.

Referring to block 302, in some embodiments, the systems and methods of the present disclosure determine a (first) plurality of variant calls using whole-genome bisulfite sequencing or targeted bisulfate sequencing of nucleic acid in a first sample from a test subject. In some such embodiments the first sample is a tissue sample.

In some embodiments, with reference to block 304, a different (second) plurality of variant calls is determined using whole-genome sequencing or targeted bisulfite sequence of nucleic acid (e.g., cell-free nucleic acid fragments) in a matched germline sample from the test subject. In some embodiments, the a matched germline sample from the test subject is whole blood.

Referring to block 306, in some embodiments, the method proceeds by removing from the first plurality of variant calls any variant call that is also in the second plurality of variant calls.

Referring to block 308, in some embodiments, the method further comprises removing from the first plurality of variant calls any variant call that is any variant call in a list of known germline variants (e.g., gnomad, dbSNP). GnomAD and dbSNP refer to reference databases of known germline variants. See Karczewski et al., 2019, “Variation across 141,456 human exomes and genomes reveals the spectrum of loss-of-function intolerance across human protein-coding genes,” bioRxiv doi.org/10.1101/531210 and Sherry et al., 2011, “dbSNP: the NCBI database of genetic variation” Nuc. Acids. Res. 29, 308-311, respectively. In some embodiments, any other known germline variants are removed from the first plurality of variant calls.

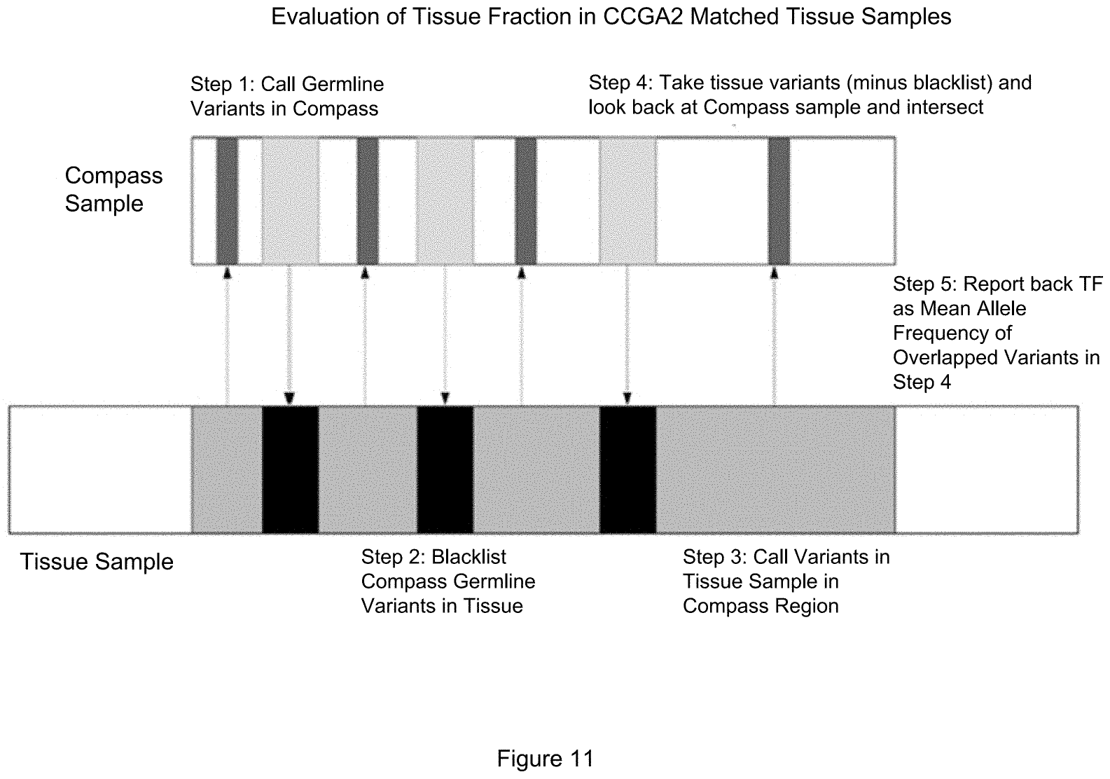

Referring to block 310, in some embodiments, the method continues by removing from the first plurality of variant calls any variant call that that has been found in a tissue sample of a subject other than the test subject (e.g., recurrent variant tissue blacklist). FIG. 11, for example, demonstrates how, in some embodiments, certain portions of a reference genome are determined to have higher information value (e.g., to be more informative in determining variants or in downstream analysis).

Referring to block 312, in some embodiments, the method further removes any variant call from the first plurality of variant calls that fails to satisfy a quality metric (e.g., minimum allele fraction, maximum allele fraction, quality of base calls (e.g. Phred scores), minimum depth, etc.).

In this way, the method identifies somatic variants through a combination of cell-free nucleic acid whole genome sequencing and biopsy whole genome bisulfite sequencing, where somatic variants are identified through analysis of the biopsy sequencing information.

Determining Whether to Call a Variant at an Allelic Position in a Test Subject.

While FIG. 3A discussed methods for pruning a plurality of variant calls for a test subject in order to ensure that such variants are somatic, as opposed to germline variants, FIGS. 3B, 3C, and 3D collectively illustrate an additional embodiment of the present disclosure that are directed to identifying variants for the test subject in the first place using methylation sequencing data from the test subject.

Blocks 202-326.

Accordingly, referring to block 320, a method of calling a variant (e.g., an SNV, insertion, deletion, or other genomic variation) at an allelic position in a test subject of a given species is provided. Referring to block 322, in some embodiments, the test subject is a human subject. In some embodiments, the test subject is a mammalian. Referring to block 326, in some embodiments, the allelic position is a single base position and the variant is a single nucleotide variant (SNV) or single nucleotide polymorphism (SNP). In some embodiments, the allelic position is two or more base positions, and the variant is an insertion or a deletion. In some embodiments, the allelic position is a portion or region of a reference genome.

Blocks 328-332.

A prior probability of genotype at the allelic position is derived (e.g., in electronic format), for each respective candidate genotype in a set of candidate genotypes, using nucleic acid data acquired from a reference population (e.g., a population of a plurality of reference subjects of the given species). With regard to block 330 in FIG. 3A, in some embodiments, the reference population comprises at least one hundred reference subjects. In some embodiments, the reference population comprises at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1000 reference subjects.

Referring to block 322, in some embodiments, each respective candidate genotype in the set of genotypes is of the form X/Y, where X is an identity of the base in the set of bases {A, C, T, G} representing one of the maternal or paternal alleles and Y is an identity of the base in the set of bases {A, C, T, G} representing the other of the maternal or paternal alleles at the allelic position in the test subject. In other words, in some embodiments, each candidate genotype in the set of genotypes represents a respective diploid genotype, and the paternal and maternal alleles at the allelic position is indicated by X and Y, respectively.

At the single nucleotide level, in some embodiments there are ten possible genotypes for each autosomal position. In some embodiments, the set of candidate genotypes consists of between two and ten genotypes in the set {A/A, A/C, A/G, A/T, C/C, C/G, C/T, G/G, G/T, and T/T}. In some embodiments, the set of candidate genotypes comprises at least two, there, four, five, six, seven, eight, or nine genotypes in the set {A/A, A/C, A/G, A/T, C/C, C/G, C/T, G/G, G/T, and T/T}. In some embodiments, the set of candidate genotypes consists of the entire set {A/A, A/C, A/G, A/T, C/C, C/G, C/T, G/G, G/T, and T/T}.

Block 334.

The method continues by obtaining (e.g., through computer system 100), for the allelic position 132, a strand-specific base count set 134 that comprises a respective forward strand base count 136 and a respective reverse strand base count 138 for each base in the set of {A, T, C, G} at the allelic position, in a forward direction and a reverse direction, which are based on determining (i) a strand orientation and (ii) an identity of a respective base at the allelic position in each respective nucleic acid fragment sequence in a corresponding plurality of nucleic acid fragment sequences that map, in electronic format, to the allelic position. In some embodiments, two or more, three or more, four or more, five or more, six or more, 10 or more, 15 or more, 20 or more, 25 or more, 30 or more, 50 or more, or 100 or more fragment sequences map to the allelic position and are accounted for in the strand-specific base count. The corresponding plurality of nucleic acid fragment sequences is acquired from a first plurality of nucleic acid fragments in a first biological sample of the test subject by methylation sequencing. In some embodiments, bases at the allelic position 132 in the nucleic acid fragment sequences whose identity can be affected by conversion of methylated or unmethylated cytosine do not contribute to the strand-specific base count set 134. In some embodiments, nucleic acid fragments are obtained as discussed in Example 2 and with reference to block 336 below.

In some embodiments, the forward direction is a F1R2 read (sense) orientation and the reverse direction is a F2R1 (antisense) read orientation. These pair of orientations refer to whether a respective nucleic acid fragment sequence originated from a 5′ or 3′ strand of the fragment for a given allelic position. For example, a F1R2 read orientation refers to a sequence read originating from a positive (sense) strand of a nucleic acid fragment, and a F2R1 read orientation refers to a sequence read originating from a negative (antisense) strand of a nucleic acid fragment. In some embodiments, the forward direction is a F1R2 or R2F1 read (sense) orientation and the reverse direction is a F2R1 or R1F2 (antisense) read orientation. See Tran et al., 2013 “Characterization of the imprinting signature of mouse embryo fibroblasts by RNA deep sequencing,” Nucleic Acids Research 42(3), 1772-1783 where this nomenclature is used.

In some embodiments, a strand-specific base count set is used to account for bisulfite conversion. Methylation sequencing inherently results in strand-specific chemistry that affects the detection of C and T alleles at the allelic position. For instance, bisulfite conversion results in a C to T conversion on the forward strand of a nucleic acid fragment and an A to G conversion on the corresponding reverse strand. Since A and G alleles are not directly affected by bisulfite conversion it is possible to resolve allele counts for the positive strand, where C and T alleles on the positive strand are identified by A and G alleles on the negative strand. As a verification, the total C and T allele count sum will be unaffected by bisulfite conversion.

Referring to block 336, in some embodiments, the first biological sample is a liquid biological sample (e.g., of the test subject) and each respective nucleic acid fragment sequence in the first plurality of nucleic acid fragment sequences represents all or a portion of a respective cell-free nucleic acid molecule in a population of cell-free nucleic acid molecules in the liquid biological sample. For instance, in some embodiments, the first biological sample comprises or consists of blood, whole blood, plasma, serum, urine, cerebrospinal fluid, fecal, saliva, sweat, tears, pleural fluid, pericardial fluid, or peritoneal fluid of the subject. In such embodiments, the first biological sample may include the blood, whole blood, plasma, serum, urine, cerebrospinal fluid, fecal, saliva, sweat, tears, pleural fluid, pericardial fluid, or peritoneal fluid of the subject as well as other components (e.g., solid tissues, etc.) of the subject.

In some embodiments, the first biological sample is a tissue biological sample (e.g., of the test subject) and each respective nucleic acid fragment sequence in the first plurality of nucleic acid fragment sequences represents all or a portion of a respective nucleic acid molecule in a population of nucleic acid molecules in the tissue sample. In some embodiments, the tissue sample is a tumor sample from the test subject. In some embodiments, the tumor sample is of a homogenous tumor. In some embodiments, the tumor sample is of a heterogenous tumor.

In some embodiments, the biological sample comprises or contains cell-free nucleic acid fragments (e.g., cfDNA fragments). In some embodiments, the biological sample is processed to extract the cell-free nucleic acids in preparation for sequencing analysis. By way of a non-limiting example, in some embodiments, cell-free nucleic acid fragments are extracted from a biological sample (e.g., blood sample) collected from a subject in K2 EDTA tubes. In the case where the biological samples are blood, in some embodiments by way of nonlimiting example, the samples are processed within two hours of collection by double spinning of the biological sample first at ten minutes at 1000 g, and then the resulting plasma is spun ten minutes at 2000 g. The plasma is then stored in 1 ml aliquots at −80° C. In this way, a suitable amount of plasma (e.g. 1-5 ml) is prepared from the biological sample for the purposes of cell-free nucleic acid extraction.

In some embodiments, cell-free nucleic acid is extracted using the QIAamp Circulating Nucleic Acid kit (Qiagen) and eluted into DNA Suspension Buffer (Sigma).

In some embodiments, the purified cell-free nucleic acid is stored at −20° C. until use. See, for example, Swanton, et al., 2017, “Phylogenetic ctDNA analysis depicts early stage lung cancer evolution,” Nature, 545(7655): 446-451, which is hereby incorporated by reference.

Other equivalent methods can be used to prepare cell-free nucleic acid from biological methods for the purpose of sequencing, and all such methods are within the scope of the present disclosure.

In some embodiments, the cell-free nucleic acid fragments that are obtained from a biological sample are any form of nucleic acid defined in the present disclosure, or a combination thereof. For example, in some embodiments, the cell-free nucleic acid that is obtained from a biological sample is a mixture of RNA and DNA.

In some embodiments, the cell-free nucleic acid fragments from a subject comprises 100 or more cell-free nucleic acid fragments, 1000 or more cell-free nucleic acid fragments, 10,000 or more cell-free nucleic acid fragments, 100,000 or more cell-free nucleic acid fragments, 1,000,000 or more cell-free nucleic acid fragments, or 10,000,000 or more nucleic acid fragments.

Sequencing of Cell-Free Nucleic Acid Fragments.

After obtaining a plurality of cell-free nucleic acid fragments from a biological sample, the cell-free nucleic acid fragments are sequenced. In some embodiments, the sequencing comprises methylation sequencing. Referring to block 338, in some embodiments, the methylation sequencing is whole-genome methylation sequencing. In some embodiments, the methylation sequencing is targeted DNA methylation sequencing using a plurality of nucleic acid probes. In some embodiments, the plurality of nucleic acid probes comprises one hundred or more probes. In some embodiments, the plurality of nucleic acid probes comprises 100 or more, 200 or more, 300 or more, 400 or more, 500 or more, 600 or more, 700 or more, 800 or more, 900 or more, 1000 or more, 2000 or more, 3000 or more, 4000 or more 5000 or more, 6000 or more, 7000 or more, 8000 or more, 9000 or more, 10,000 or more, 25,000 or more, or 50,000 or more probes. In some embodiments, some or all of the probes uniquely map to a genomic region described in International Patent Publication No. WO2020154682A3, entitled “Detecting Cancer, Cancer Tissue or Origin, or Cancer Type,” which is hereby incorporated by reference, including the Sequence Listing referenced therein. In some embodiments, some or all of the probes uniquely map to a genomic region described in International Patent Publication No. WO2020/069350A1, entitled “Methylated Markers and Targeted Methylation Probe Panel,” which is hereby incorporated by reference, including the Sequence Listing referenced therein. In some embodiments, some or all of the probes uniquely map to a genomic region described in International Patent Publication No. WO2019/195268A2, entitled “Methylated Markers and Targeted Methylation Probe Panels,” which is hereby incorporated by reference, including the Sequence Listing referenced therein.

In some embodiments, the methylation sequencing detects one or more 5-methylcytosine (5mC) and/or 5-hydroxymethylcytosine (5hmC) in respective nucleic acid fragments in the first plurality of nucleic acid fragments. In some embodiments, the methylation sequencing comprises the conversion of one or more unmethylated cytosines or one or more methylated cytosines, in the nucleic acid fragments in the first plurality of nucleic acid fragments, to a corresponding one or more uracils. In some embodiments, the one or more uracils are converted during amplification and detected during the methylation sequencing as one or more corresponding thymines. In some embodiments, the conversion of one or more unmethylated cytosines or one or more methylated cytosines comprises a chemical conversion, an enzymatic conversion, or combinations thereof.

In some such embodiments, prior to sequencing the cell-free nucleic acid fragments are treated to convert unmethylated cytosines to uracils. In some embodiments, the method uses a bisulfite treatment of the DNA that converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, Calif.)) is used for the bisulfite conversion in some embodiments. In some embodiments, the conversion of unmethylated cytosines to uracils is accomplished using an enzymatic reaction. For example, the conversion can use a commercially available kit for the conversion of unmethylated cytosines to uracils, such as APOBEC-Seq (NEBiolabs, Ipswich, Mass.).

From the converted cell-free nucleic acid fragments, a sequencing library is prepared. Optionally, the sequencing library is enriched for cell-free nucleic acid fragments, or genomic regions, that are informative for cell origin using a plurality of hybridization probes, such as any combination of regions disclosed in, for example, International Patent Publication No. WO2020154682A3, entitled “Detecting Cancer, Cancer Tissue or Origin, or Cancer Type,” International Patent Publication No. WO2020/069350A1, entitled “Methylated Markers and Targeted Methylation Probe Panel,” and/or International Patent Publication No. WO2019/195268A2, entitled “Methylated Markers and Targeted Methylation Probe Panels,” each of which is hereby incorporated by reference. In some embodiments, the hybridization probes are short oligonucleotides that hybridize to particularly specified cell-free nucleic acid fragments, or targeted regions, and enrich for those fragments or regions for subsequent sequencing and analysis as disclosed in for example, International Patent Publication No. WO2020154682A3, entitled “Detecting Cancer, Cancer Tissue or Origin, or Cancer Type,” International Patent Publication No. WO2020/069350A1, entitled “Methylated Markers and Targeted Methylation Probe Panel,” and/or International Patent Publication No. WO2019/195268A2, entitled “Methylated Markers and Targeted Methylation Probe Panels,” each of which is hereby incorporated by reference. In some embodiments, hybridization probes are used to perform targeted, high-depth analysis of a set of specified CpG sites that are informative for cell origin. Once prepared, the sequencing library or a portion thereof is sequenced to obtain a plurality of sequence reads.

In this way, in some embodiments, more than 1000, 5000, 10,000, 50,000, 100,000, 200,000, 500,000, 1×10⁶, 1×10⁷, or more than 1×10⁸ sequence reads are recovered from the biological sample. In some embodiments, the sequence reads recovered from the biological sample provide an average coverage rate of 1× or greater, 2× or greater, 5× or greater, 10× or greater, 20× or greater, 30× or greater, 40× or greater, 50× or greater, 100× or greater, or 200× or greater across at least two percent, at least five percent, at least ten percent, at least twenty percent, at least thirty percent, at least forty percent, at least fifty percent, at least sixty percent, at least seventy percent, at least eighty percent, at least ninety percent, at least ninety-eight percent, or at least ninety-nine percent of the genome of the subject. In embodiments where the biological sample comprises or contains cell-free nucleic acid fragments, the resulting sequence reads are thus of cell-free nucleic acid fragments in the biological sample.

In some embodiments, any form of sequencing can be used to obtain the sequence reads from the cell-free nucleic acid fragments obtained from the biological sample. Example sequencing methods include, but are not limited to, high-throughput sequencing systems such as the Roche 454 platform, the Applied Biosystems SOLID platform, the Helicos True Single Molecule DNA sequencing technology, the sequencing-by-hybridization platform from Affymetrix Inc., the single-molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and nanopore sequencing also can be used to obtain sequence reads from the cell-free nucleic acid obtained from the biological sample.

In some embodiments, sequencing-by-synthesis and reversible terminator-based sequencing (e.g., Illumina's Genome Analyzer; Genome Analyzer II; HISEQ 2000; HISEQ 2500 (Illumina, San Diego Calif.)) is used to obtain sequence reads from the cell-free nucleic acid obtained from the biological sample. In some such embodiments, millions of cell-free nucleic acid (e.g., DNA) fragments are sequenced in parallel. In one example of this type of sequencing technology, a flow cell is used that contains an optically transparent slide with eight individual lanes on the surfaces of which are bound oligonucleotide anchors (e.g., adaptor primers). A flow cell often is a solid support that is configured to retain and/or allow the orderly passage of reagent solutions over bound analytes. In some instances, flow cells are planar in shape, optically transparent, generally in the millimeter or sub-millimeter scale, and often have channels or lanes in which the analyte/reagent interaction occurs. In some embodiments, a cell-free nucleic acid sample can include a signal or tag that facilitates detection. In some such embodiments, the acquisition of sequence reads from the cell-free nucleic acid obtained from the biological sample includes obtaining quantification information of the signal or tag via a variety of techniques such as, for example, flow cytometry, quantitative polymerase chain reaction (qPCR), gel electrophoresis, gene-chip analysis, microarray, mass spectrometry, cytofluorimetric analysis, fluorescence microscopy, confocal laser scanning microscopy, laser scanning cytometry, affinity chromatography, manual batch mode separation, electric field suspension, sequencing, and combination thereof.

In some embodiments, the sequence reads are corrected for background copy number. For instance, sequence reads that arise from chromosomes or portions of chromosomes that are duplicated in the subject are corrected for this duplication. This can be done by normalizing before running this inference.

Whole Genome Bisulfite Sequencing Assay.

In some embodiments, the subject is human and the sequence reads are obtained through bisulfite sequencing and are evaluated for methylation status on a genome-wide basis. In some embodiments, the whole-genome bisulfite sequencing assay looks for variations in methylation patterns in the genome. See, for example, Example 6. See also, United States Patent Publication No. US 2019-0287652 A1, entitled “Anomalous Fragment Detection and Classification,” which is hereby incorporated by reference.

Block 340.

Referring to block 340 of FIG. 3C, in some embodiments, the systems and methods of the present disclosure compute a respective forward strand conditional probability and a respective reverse strand conditional probability for each respective candidate genotype in the set of candidate genotypes for the allelic position using the strand-specific base count set and a sequencing error estimate thereby computing a plurality of forward strand conditional probabilities and a plurality of reverse strand conditional probabilities for the allelic position.

Referring to block 342, in some embodiments, the sequencing error estimate is between 0.01 and 0.0001. In some embodiments, the sequencing error estimate is less than 0.01, less than 0.009, less than 0.008, less than 0.007, less than 0.006, less than 0.005, less than 0.004, less than 0.003, less than 0.002, less than 0.001, less than 0.00075, less than 0.0005, or less than 0.0075. In some embodiments, a respective sequencing error estimate is used for each candidate genotype in the set of candidate genotypes. In some embodiments, the same sequencing error estimate is used for each candidate genotypes in the set of candidate genotypes. In some embodiments, one or more of the candidate genotypes has a corresponding sequencing error estimate that is distinct from the sequencing error estimate used for the remaining candidate genotypes in the set of candidate genotypes. In some embodiments, symmetric error estimates are assumed for each genotype.

In some embodiments, for example for calling germline variants, the sequencing error (e.g., c) is fixed at a constant value between 0.1 and 0.9, such as 0.5. In some embodiments, for example for somatic variant calling, the sequencing error estimate is allowed to vary.

Block 344.

Referring to block 344 of FIG. 3C, in some embodiments, the systems and methods of the present disclosure compute a plurality of likelihoods for an allelic position. Each respective likelihood in the plurality of likelihoods is for a respective candidate genotype in the set of candidate genotypes. In some embodiments, the plurality of likelihoods are computed using a combination of (i) the respective forward strand conditional probability for the respective candidate genotype in the plurality of forward strand conditional probabilities, (ii) the respective reverse strand conditional probability for the respective candidate genotype in the plurality of reverse strand conditional probabilities, and (iii) the prior probability of genotype for the respective candidate genotype.

In some embodiments, Bayes' theorem is used to compute the likelihood of observing a respective genotype. In some embodiments, the prior likelihood for each respective genotype is calculated using observed allele frequencies. In some embodiments, each candidate genotype in the set of candidate genotypes for an allelic position is ranked in order of respective Bayesian probability.

In some embodiments, a respective likelihood for a respective candidate genotype in the set of candidate genotypes is represented as:

Pr(F_{A},F_{G},F_{CT}|F_{ACGT},genotype,ϵ)*Pr(R_{AG},R_{C},R_{T}|R_{ACGT},genotype,ϵ)*Pr(G)

where Pr(F_{A}, F_{G}, F_{CT}|F_{ACGT}, genotype, ϵ) is the respective forward strand conditional probability for the respective candidate genotype, Pr(R_{C}, R_{T}, R_{AG}|R_{ACGT}, genotype, ϵ) is the respective reverse strand conditional probability for the respective candidate genotype, Pr(G) is the prior probability of genotype at the allelic position for the respective candidate genotype, ϵ is the sequencing error estimate, genotype refers to the respective candidate genotype, F_{A} is the forward direction base count for base A at the allelic position across the first plurality of nucleic acid fragment sequences that map to the allelic position from the first biological sample, in the strand specific base count set, F_{G} is the forward direction base count for base G at the allelic position across the first plurality of nucleic acid fragment sequences that map to the allelic position from the first biological sample, in the strand specific base count set, F_{CT} is a summation of (i) the forward direction base count for base C and (ii) the forward direction base count for base T at the allelic position across the first plurality of nucleic acid fragment sequences that map to the allelic position from the first biological sample, in the strand specific base count set, R_{C} is the reverse direction base count for base C at the allelic position across the first plurality of nucleic acid fragment sequences that map to the allelic position from the first biological sample, in the strand specific base count set, R_{T} is the reverse direction base count for base T at the allelic position across the first plurality of nucleic acid fragment sequences that map to the allelic position from the first biological sample, in the strand specific base count set, and R_{AG} is a summation of (i) the reverse direction base count for base A and (ii) the reverse direction base count for base G at the allelic position across the first plurality of nucleic acid fragment sequences that map to the allelic position from the first biological sample, in the strand specific base count set.

In some embodiments, this multiplication depends on the assumption of symmetric sequencing error estimates for each candidate genome. In some embodiments, the likelihood is a log-likelihood, which is determined by taking the log of the above-defined equation.

In some embodiments, the respective candidate genotype G is A/A and computing the respective likelihood:

Pr(F_{A},F_{G},F_{CT}|F_{ACGT},genotype,ϵ)*Pr(R_{AG},R_{G},R_{T}|R_{ACGT},genotype,ϵ)*Pr(A/A),

for A/A comprises calculating:

$$[ {( 1 - ϵ )}^{{F}_{A}} {( \frac{ϵ}{3} )}^{{F}_{G}} {( \frac{2 ϵ}{3} )}^{{F}_{CT}} ] * [ {( 1 - \frac{2 ϵ}{3} )}^{{R}_{AG}} {( \frac{ϵ}{3} )}^{{R}_{C}} {( \frac{ϵ}{3} )}^{{R}_{T}} ] * Pr ( A \text{/} A ) .$$

In some embodiments, the respective candidate genotype G is A/A and computing the respective likelihood:

Pr(F_{A},F_{G},F_{CT}|F_{ACGT},genotype,ϵ)*Pr(R_{AG},R_{C},R_{T}|R_{ACGT},genotype,ϵ)*Pr(A/A),

for A/A comprises calculating the log-likelihood:

$${log ( 1 - ϵ )}^{{F}_{A}} + {log ( \frac{ϵ}{3} )}^{{F}_{G}} + {log ( \frac{2 ϵ}{3} )}^{{F}_{CT}} + {log ( 1 - \frac{2 ϵ}{3} )}^{{R}_{AG}} + {log ( \frac{ϵ}{3} )}^{{R}_{C}} + {log ( \frac{ϵ}{3} )}^{{R}_{T}} + log ( Pr ( A \text{/} A ) ) .$$

In some embodiments, the respective candidate genotype G is A/C and computing the respective likelihood:

Pr(F_{A},F_{G},F_{CT}|F_{ACGT},genotype,ϵ)*Pr(R_{AG},R_{C},R_{T}|R_{ACGT},genotype,ϵ)*Pr(A/C),

for A/C comprises calculating:

$$[ {( 0.5 - \frac{ϵ}{3} )}^{{F}_{A}} {( \frac{ϵ}{3} )}^{{F}_{G}} {( 0.5 )}^{{F}_{CT}} ] * [ {( 0.5 )}^{{R}_{AG}} {( 0.5 - \frac{ϵ}{3} )}^{{R}_{C}} {( \frac{ϵ}{3} )}^{{R}_{T}} ] * Pr ( A \text{/} C ) .$$

In some embodiments, the respective candidate genotype is G is A/C and computing the respective likelihood:

Pr(F_{A},F_{G},F_{CT}|F_{ACGT},genotype,ϵ)*Pr(R_{AG},R_{C},R_{T}|R_{ACGT},genotype,ϵ)*Pr(A/C),

for A/C comprises calculating the log-likelihood:

$${log ( 0.5 - \frac{ϵ}{3} )}^{{F}_{A}} + {log ( \frac{ϵ}{3} )}^{{F}_{G}} + {log ( 0.5 )}^{{F}_{CT}} + {log ( 0.5 )}^{{R}_{AG}} + {log ( 0.5 - \frac{ϵ}{3} )}^{{R}_{C}} + {log ( \frac{ϵ}{3} )}^{{R}_{T}} + log ( Pr ( A \text{/} C ) ) .$$

In some embodiments, the respective candidate genotype is G is A/G and computing the respective likelihood:

Pr(F_{A},F_{G},F_{CT}|F_{ACGT},genotype,ϵ)*Pr(R_{AG},R_{C},R_{T}|R_{ACGT},genotype,ϵ)*Pr(A/G),

for A/G comprises calculating:

$$[ {( 0.5 - \frac{ϵ}{3} )}^{{F}_{A}} {( 0.5 - \frac{ϵ}{3} )}^{{F}_{G}} {( \frac{2 ϵ}{3} )}^{{F}_{CT}} ] * [ {( 1 - \frac{2 ϵ}{3} )}^{{R}_{AG}} {( \frac{ϵ}{3} )}^{{R}_{C}} {( \frac{ϵ}{3} )}^{{R}_{T}} ] * Pr ( A \text{/} G ) .$$

In some embodiments, the respective candidate genotype G is A/G and computing the respective likelihood:

Pr(F_{A},F_{G},F_{CT}|F_{ACGT},genotype,ϵ)*Pr(R_{AG},R_{C},R_{T}|R_{ACGT},genotype,ϵ)*Pr(A/G),

for A/G comprises calculating the log-likelihood:

$$log {( 0.5 - \frac{ϵ}{3} )}^{{F}_{A}} + log {( 0.5 - \frac{ϵ}{3} )}^{{F}_{G}} + log {( \frac{2 ϵ}{3} )}^{{F}_{C T}} + {log ( 1 - \frac{2 ϵ}{3} )}^{{R}_{A G}} + {log ( \frac{ϵ}{3} )}^{{R}_{C}} + log {( \frac{ϵ}{3} )}^{{R}_{T}} + log ( P r ( A / G ) ) .$$

In some embodiments, the respective candidate genotype G is A/T and computing the respective likelihood:

Pr(F_{A},F_{G},F_{CT}|F_{ACGT},genotype,ϵ)*Pr(R_{AG},R_{C},R_{T}|R_{ACGT},genotype,ϵ)*Pr(A/T),

for A/T comprises calculating:

$$[ {( 0.5 - \frac{ϵ}{3} )}^{{F}_{A}} {( \frac{ϵ}{3} )}^{{F}_{G}} {( 0.5 )}^{{F}_{C T}} ] * [ {( 0.5 )}^{{R}_{A G}} {( \frac{ϵ}{3} )}^{{R}_{C}} {( 0.5 - \frac{ϵ}{3} )}^{{R}_{T}} ] * P r ( A / T ) .$$

In some embodiments, the respective candidate genotype G is A/T and computing the respective likelihood:

Pr(F_{A},F_{G},F_{CT}|F_{ACGT},genotype,ϵ)*Pr(R_{AG},R_{C},R_{T}|R_{ACGT},genotype,ϵ)*Pr(A/T),

for A/T comprises calculating the log-likelihood:

$$log {( 0.5 - \frac{ϵ}{3} )}^{{F}_{A}} + log {( \frac{ϵ}{3} )}^{{F}_{G}} + log {( 0.5 )}^{{F}_{C T}} + log {( 0.5 )}^{{R}_{A G}} + log {( \frac{ϵ}{3} )}^{{R}_{C}} + log {( 0.5 - \frac{ϵ}{3} )}^{{R}_{T}} + log ( P r ( A / T ) ) .$$

In some embodiments, the respective candidate genotype G is C/C and computing the respective likelihood:

Pr(F_{A},F_{G},F_{CT}|F_{ACGT},genotype,ϵ)*Pr(R_{AG},R_{C},R_{T}|R_{ACGT},genotype,ϵ)*Pr(C/C),

for C/C comprises calculating:

$$[ {( \frac{ϵ}{3} )}^{{F}_{A}} {( \frac{ϵ}{3} )}^{{F}_{G}} {( 1 - \frac{2 ϵ}{3} )}^{{F}_{C T}} ] * [ {( \frac{2 ϵ}{3} )}^{{R}_{A G}} {( 1 - ϵ )}^{{R}_{C}} {( \frac{ϵ}{3} )}^{{R}_{T}} ] * P r ( C / C ) .$$

In some embodiments, the respective candidate genotype G is C/C and computing the respective likelihood:

Pr(F_{A},F_{G},F_{CT}|F_{ACGT},genotype,ϵ)*Pr(R_{AG},R_{C},R_{T}|R_{ACGT},genotype,ϵ)*Pr(C/C),

for C/C comprises calculating the log-likelihood:

$$log {( \frac{ϵ}{3} )}^{{F}_{A}} + log {( \frac{ϵ}{3} )}^{{F}_{G}} + log {( 1 - \frac{2 ϵ}{3} )}^{{F}_{C T}} + {log ( \frac{2 ϵ}{3} )}^{{R}_{A G}} + log {( 1 - ϵ )}^{{R}_{C}} + log {( \frac{ϵ}{3} )}^{{R}_{T}} + log ( P r ( C / C ) ) .$$

In some embodiments, the respective candidate genotype G is C/G and computing the respective likelihood:

Pr(F_{A},F_{G},F_{CT}|F_{ACGT},genotype,ϵ)*Pr(R_{AG},R_{C},R_{T}|R_{ACGT},genotype,ϵ)*Pr(C/G),

for C/G comprises calculating:

$$[ {( \frac{ϵ}{3} )}^{{F}_{A}} {( 0.5 - \frac{ϵ}{3} )}^{{F}_{G}} {( 0.5 )}^{{F}_{C T}} ] * [ {( 0.5 )}^{{R}_{A G}} {( 0.5 - \frac{ϵ}{3} )}^{{R}_{C}} {( \frac{ϵ}{3} )}^{{R}_{T}} ] * P r ( C / G ) .$$

In some embodiments, the respective candidate genotype G is C/G and computing the respective likelihood:

Pr(F_{A},F_{G},F_{CT}|F_{ACGT},genotype,ϵ)*Pr(R_{AG},R_{C},R_{T}|R_{ACGT},genotype,ϵ)*Pr(C/G),

for C/G comprises calculating the log-likelihood:

$$log {( \frac{ϵ}{3} )}^{{F}_{A}} + log {( 0.5 - \frac{ϵ}{3} )}^{{F}_{G}} + log {( 0.5 )}^{{F}_{C T}} + log {( 0.5 )}^{{R}_{A G}} + log {( 0.5 - \frac{ϵ}{3} )}^{{R}_{C}} + log {( \frac{ϵ}{3} )}^{{R}_{T}} + log ( P r ( C / G ) ) .$$

In some embodiments, the respective candidate genotype G is C/T and computing the respective likelihood:

Pr(F_{A},F_{G},F_{CT}|F_{ACGT},genotype,ϵ)*Pr(R_{AG},R_{C},R_{T}|R_{ACGT},genotype,ϵ)*Pr(C/T),

for C/T comprises calculating:

$$[ {( \frac{ϵ}{3} )}^{{F}_{A}} {( \frac{ϵ}{3} )}^{{F}_{G}} {( 1 - \frac{2 ϵ}{3} )}^{{F}_{C T}} ] * [ {( \frac{2 ϵ}{3} )}^{{R}_{A G}} {( 0.5 - \frac{ϵ}{3} )}^{{R}_{C}} {( 0.5 - \frac{ϵ}{3} )}^{{R}_{T}} ] * P r ( C / T ) .$$

In some embodiments, the respective candidate genotype G is C/T and computing the respective likelihood:

Pr(F_{A},F_{G},F_{CT}|F_{ACGT},genotype,ϵ)*Pr(R_{AG},R_{C},R_{T}|R_{ACGT},genotype,ϵ)*Pr(C/T),

for C/T comprises calculating the log-likelihood:

$$log {( \frac{ϵ}{3} )}^{{F}_{A}} + log {( \frac{ϵ}{3} )}^{{F}_{G}} + log {( 1 - \frac{2 ϵ}{3} )}^{{F}_{C T}} + {log ( \frac{2 ϵ}{3} )}^{{R}_{A G}} + log {( 0.5 - \frac{ϵ}{3} )}^{{R}_{C}} + log {( 0.5 - \frac{ϵ}{3} )}^{{R}_{T}} + log ( P r ( C / T ) ) .$$

In some embodiments, the respective candidate genotype G is G/G and computing the respective likelihood:

Pr(F_{A},F_{G},F_{CT}|F_{ACGT},genotype,ϵ)*Pr(R_{AG},R_{C},R_{T}|R_{ACGT},genotype,ϵ)*Pr(G/G),

for G/G comprises calculating:

$$[ {( \frac{ϵ}{3} )}^{{F}_{A}} {( 1 - ϵ )}^{{F}_{G}} {( \frac{2 ϵ}{3} )}^{{F}_{C T}} ] * [ {( 1 - \frac{2 ϵ}{3} )}^{{R}_{A G}} {( \frac{ϵ}{3} )}^{{R}_{C}} {( \frac{ϵ}{3} )}^{{R}_{T}} ] * P r ( G / G ) .$$

In some embodiments, the respective candidate genotype G is G/G and computing the respective likelihood:

Pr(F_{A},F_{G},F_{CT}|F_{ACGT},genotype,ϵ)*Pr(R_{AG},R_{C},R_{T}|R_{ACGT},genotype,ϵ)*Pr(G/G),

for G/G comprises calculating the log-likelihood:

$$log {( \frac{ϵ}{3} )}^{{F}_{A}} + log {( 1 - ϵ )}^{{F}_{G}} + log {( \frac{2 ϵ}{3} )}^{{F}_{C T}} + {log ( 1 - \frac{2 ϵ}{3} )}^{{R}_{A G}} + log {( \frac{ϵ}{3} )}^{{R}_{C}} + log {( \frac{ϵ}{3} )}^{{R}_{T}} + log ( P r ( G / G ) ) .$$

In some embodiments, the respective candidate genotype G is G/T and computing the respective likelihood:

Pr(F_{A},F_{G},F_{CT}|F_{ACGT},genotype,ϵ)*Pr(R_{AG},R_{C},R_{T}|R_{ACGT},genotype,ϵ)*Pr(G/T),

for G/T comprises calculating:

$$[ {( \frac{ϵ}{3} )}^{{F}_{A}} {( 0.5 - \frac{ϵ}{3} )}^{{F}_{G}} {( 0.5 )}^{{F}_{C T}} ] * [ {( 0.5 )}^{{R}_{A G}} {( \frac{ϵ}{3} )}^{{R}_{C}} {( 0.5 - \frac{ϵ}{3} )}^{{R}_{T}} ] * P r ( G / T ) .$$

In some embodiments, the respective candidate genotype G is G/T and computing the respective likelihood:

Pr(F_{A},F_{G},F_{CT}|F_{ACGT},genotype,ϵ)*Pr(R_{AG},R_{C},R_{T}|R_{ACGT},genotype,ϵ)*Pr(G/T),

for G/T comprises calculating the log-likelihood:

$$log {( \frac{ϵ}{3} )}^{{F}_{A}} + log {( 0.5 - \frac{ϵ}{3} )}^{{F}_{G}} + log {( 0.5 )}^{{F}_{C T}} + log {( 0.5 )}^{{R}_{A G}} + log {( \frac{ϵ}{3} )}^{{R}_{C}} + log {( 0.5 - \frac{ϵ}{3} )}^{{R}_{T}} + log ( P r ( G / T ) ) .$$

In some embodiments, the respective candidate genotype G is T/T and computing the respective likelihood:

Pr(F_{A},F_{G},F_{CT}|F_{ACGT},genotype,ϵ)*Pr(R_{AG},R_{C},R_{T}|R_{ACGT},genotype,ϵ)*Pr(T/T),

for T/T comprises calculating:

$$[ {( \frac{ϵ}{3} )}^{{F}_{A}} {( \frac{ϵ}{3} )}^{{F}_{G}} {( 1 - \frac{2 ϵ}{3} )}^{{F}_{C T}} ] * [ {( \frac{2 ϵ}{3} )}^{{R}_{A G}} {( \frac{ϵ}{3} )}^{{R}_{C}} {( 1 - ϵ )}^{{R}_{T}} ] * P r ( T / T ) .$$

In some embodiments, the respective candidate genotype G is T/T and computing the respective likelihood:

Pr(F_{A},F_{G},F_{CT}|F_{ACGT},genotype,ϵ)*Pr(R_{AG},R_{G},R_{T}|R_{ACGT},genotype,ϵ)*Pr(T in,

for T/T comprises calculating the log-likelihood:

$${log ( \frac{ϵ}{3} )}^{{F}_{A}} + log {( \frac{ϵ}{3} )}^{{F}_{G}} + log {( 1 - \frac{2 ϵ}{3} )}^{{F}_{C T}} + {log ( \frac{2 ϵ}{3} )}^{{R}_{A G}} + log {( \frac{ϵ}{3} )}^{{R}_{C}} + log {( ( 1 - ϵ ) )}^{{R}_{T}} + log ( P r ( T / T ) ) .$$

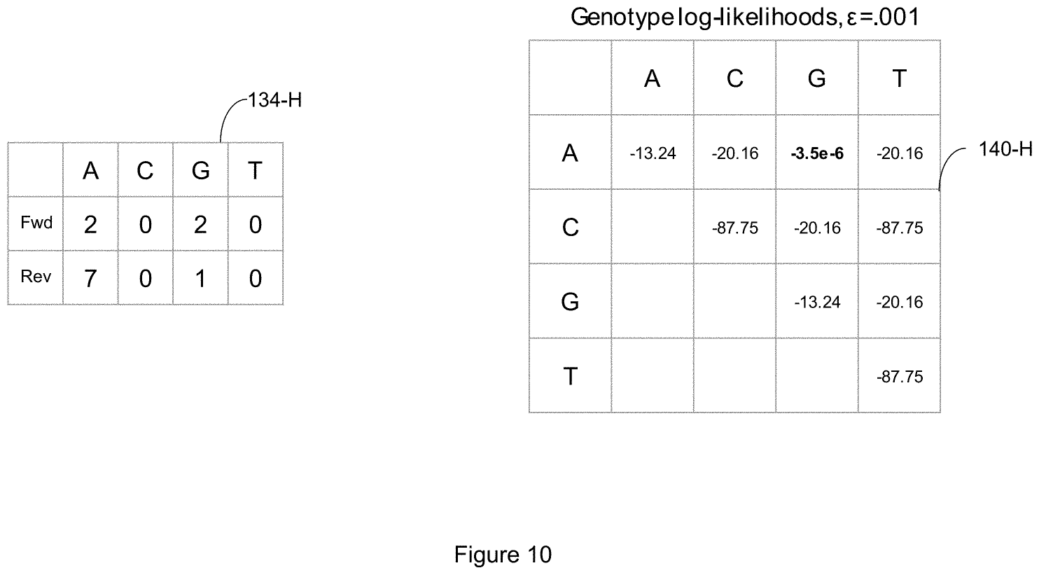

FIG. 10 provides an example of the conversion from a respective base count set 134-H to a corresponding set of candidate genotype log-likelihoods 140-H, in accordance with the calculations described above for each candidate genotype.

In some embodiments, one or more respective likelihood calculations further includes a corresponding bisulfite-conversion-rate prior to account for apparent disparities between the counts of C on corresponding forward and reverse strands. For example, if a higher number of C bases are observed on a forward strand, that would suggest that a T/T is ultimately less likely than a C/T of C/C genotype. Examples of likelihood calculations that account for bisulfite conversion rates, base quality scores, and other sequencing information are provided in Liu et al. 2012 “Bis-SNP: Combined DNA methylation and SNP calling for Bisulfite-seq data,” Genome Biol. 13(7), R61, which is hereby incorporated by reference in entirety.

Block 346.

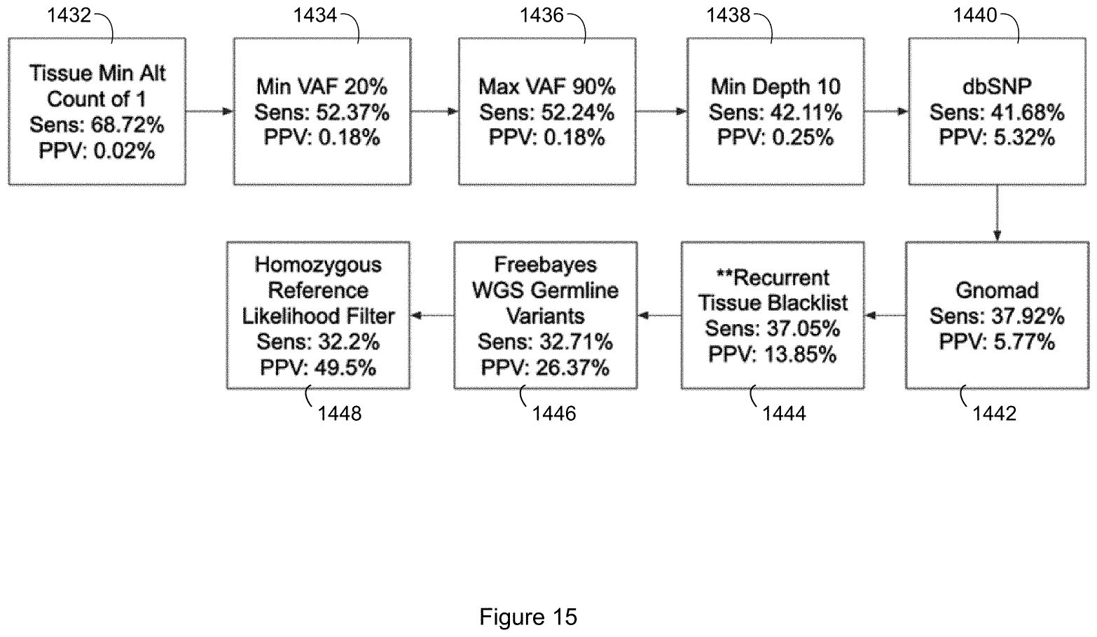

Referring to block 346 of FIG. 3C, in some embodiments, the systems and methods of the present disclosure determine whether the plurality of likelihoods computed in block 344 supports a variant call at the allelic position. In some embodiments, this comprises determining whether any likelihood in the plurality of likelihoods for any of the proposed genotypes for the allelic position satisfies a variant threshold. In some embodiments, when a likelihood for any of the proposed genotypes for the allelic position satisfies a variant threshold, a variant at the allelic position is called. Thus, from among the plurality of likelihoods corresponding to a plurality of different variant alleles, a variant allele is called from among the plurality of different variant alleles if the likelihood for the variant allele satisfies a threshold value. If more than two variant alleles satisfies the threshold value, than one with the greatest likelihood below the threshold is called. If none of the variant alleles satisfies the threshold value, no variant allele is called. Block 346 thus represents filter 1448 of FIG. 15.

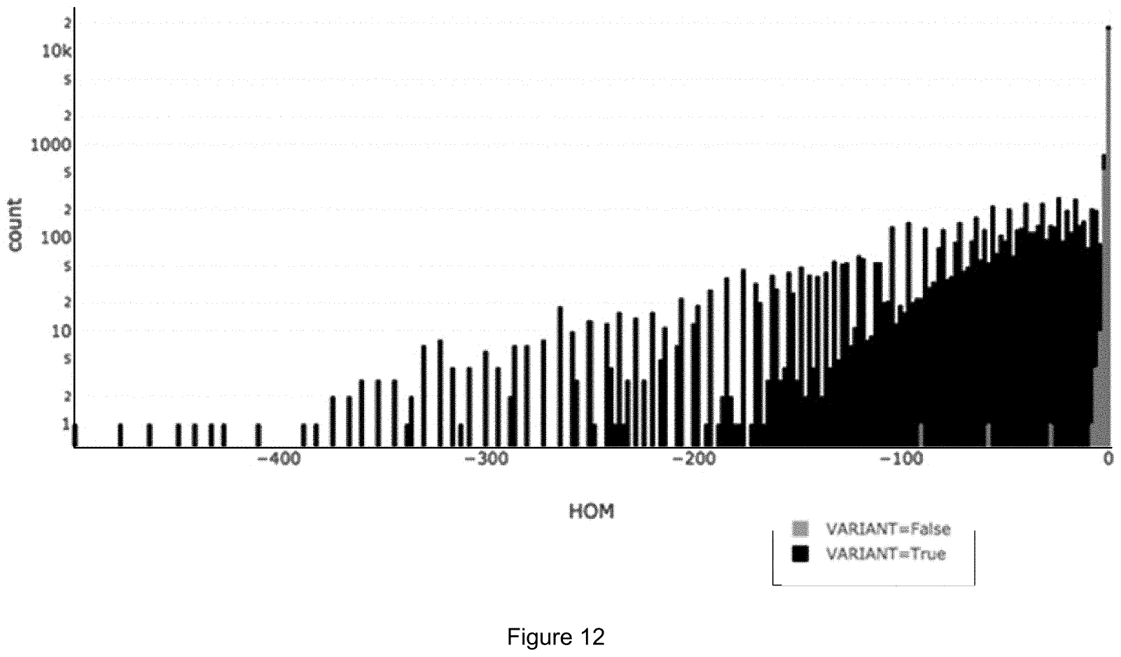

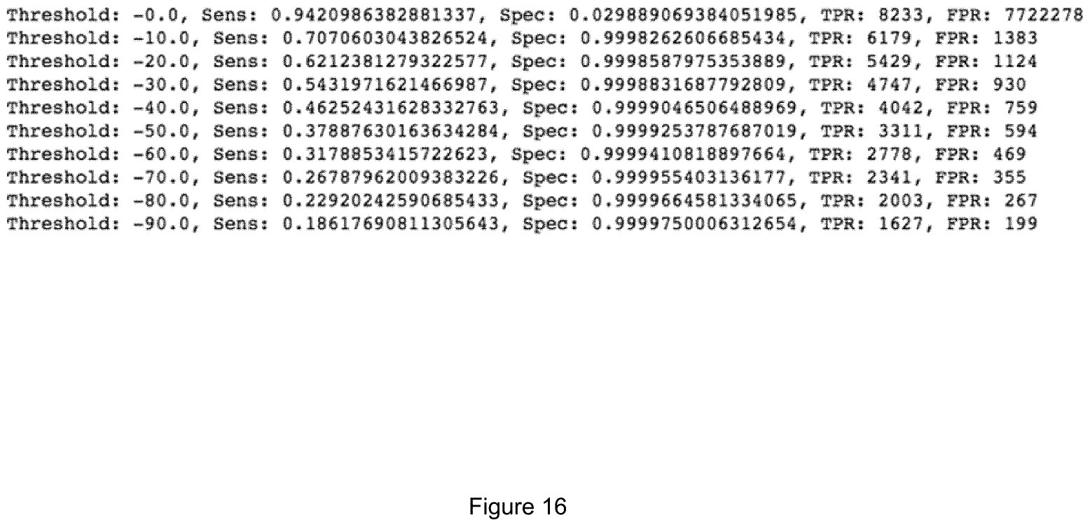

In FIG. 12, filtering of candidate variants is demonstrated with a threshold for the homozygous allele A/A of the reference allele ‘A.’ In FIG. 12, if a candidate variant has a likelihood below the threshold, it is determined to be a variant. The ultimate variant call is determined to be the variant with the highest likelihood (e.g., the maximum of A/C, A/G, and A/T for reference allele A). FIG. 16 show the sensitivity (Sens), specificity (Spec), true positive rate (TPR), and false positive rate (FPR) for threshold values of 0, −10, −20, −30, −40, −50, −60, −70, −80 and −90 using a paired whole genome bisulfite sequencing (WGBS)/whole genome sequencing (WGS) sequencing data described in Example 5. Thus, from at least the data used for FIG. 16, an empirical threshold of −10 for the genotype log-likelihood (as calculated in FIG. 10) provides the best performance. However, for other datasets other thresholds may be applicable. In some embodiments, the plurality of reference subjects (whose genotypes determine the variant threshold) comprises at least ten reference subjects. In some embodiments, the plurality of reference subjects comprises at least one hundred reference subjects. In some embodiments, the plurality of reference subjects comprises at least 10 reference subjects, at least 25 reference subjects, at least 50 reference subjects, at least 75 reference subjects, at least 100 reference subjects, at least 200 reference subjects, or at least 500 reference subjects. Moreover, in some embodiments, rather than using a threshold cutoff on log-likelihood or likelihood for filter 1448 a classifier that takes as input (i) the strand-specific base count set 134 (comprising the respective forward strand base count 136 and the respective reverse strand base count 138 for each base in the set of {A, T, C, G} at the allelic position, in the forward and reverse direction), and (ii) the prior probability of genotype for the respective candidate genotype to call the allelic position is used. In some embodiments this classifier is one or more neural networks, support vector machines, Naive Bayes classifiers, nearest neighbor classifiers, boosted trees classifier, random forest classifiers, decision tree classifiers, multinomial logistic regression classifiers, linear models, linear regression classifiers, or ensembles thereof.

In some embodiments, the likelihood is expressed as a log-likelihood (e.g., an unnormalized likelihood) and the variant threshold is satisfied when the log-likelihood for the reference genotype for the allelic position is less than −10. In some embodiments, a variant threshold is satisfied when the log-likelihood for the reference genotype for the allelic position is less than −1, less than −5, less than −10, less than −25, less than −50, or less than −100. In some embodiments, the likelihood is expressed as a log-likelihood and the variant threshold is satisfied when the log-likelihood for the reference genotype for the allelic position is between −25 and −5. In some embodiments, the likelihood is expressed as a log-likelihood and the variant threshold is satisfied when the log-likelihood for the reference genotype for the allelic position is between −10 and −1, between −10 and −5, between −25 and −1, between −25 and −10, between −25 and −15, between −50 and −1, between −50 and −5, between −50 and −10, or between −50 and −25.

In some embodiments, the likelihood is expressed as a normalized likelihood (e.g., a respective posterior probability for each reference genotype). For example, in some such embodiments, each reference genotype has a distinct normalized likelihood. In some embodiments, two or more reference genotypes have the same normalized likelihood. In some embodiments, the variant threshold is satisfied when the normalized likelihood for the reference genotype for the allelic position is less than −1, less than −5, less than −10, less than −25, less than −50, or less than −100. In some embodiments, the variant threshold is satisfied when the normalized likelihood for the reference genotype for the allelic position is between −10 and −1, between −10 and −5, between −25 and −1, between −25 and −10, between −25 and −15, between −50 and −1, between −50 and −5, between −50 and −10, or between −50 and −25.

In some embodiments, the systems and methods of the present disclosure further determine, when a variant at the allelic position is called, an identity of the variant by selecting the candidate genotype in the set of candidate genotypes for the allelic position that has the best likelihood in the plurality of likelihoods as the variant. In some embodiments, this determination requires ranking the candidate genotypes by their corresponding likelihoods or log-likelihoods.

In some embodiments, the reference genotype for the allelic position is homozygous (e.g., A/A, T/T, G/G, C/C).

Block 348.

In some embodiments, the systems and methods of the present disclosure further repeat the method for each allelic position in a plurality of allelic positions for the test subject (e.g., thereby obtaining a plurality of variant calls for the test subject). In some such embodiments, repeating the method comprises performing the obtaining a respective prior probability of genotype (e.g. blocks 328-332), obtaining a respective strand-specific base count set (e.g., blocks 334-338), computing a respective forward strand conditional probability and a respective reverse strand conditional probability (e.g., blocks 340-342), computing a respective plurality of likelihoods (e.g., block 344), and determining whether the respective plurality of likelihoods (or log-likelihoods) supports a respective variant call (e.g., block 346), for each allelic position in a plurality of allelic positions, thereby obtaining a plurality of variant calls for the test subject, where each variant call in the plurality of variant calls is at a different genomic position in a reference genome. In some such embodiments, the first biological sample is a tissue sample, and the methylation sequencing is whole-genome bisulfite sequencing. In some such embodiments, the first biological sample is a tissue sample, and the methylation sequencing is targeted bisulfite sequencing. Referring to block 350, in some embodiments the first biological sample is a tissue sample, and the methylation sequencing is whole genome bisulfite sequencing.

In some embodiments, the plurality of variant calls comprises 200 variant calls. In some embodiments, the plurality of variant calls comprises at least 10 variant calls, at least 20 variant calls, at least 30 variant calls, at least 40 variant calls, at least 50 variant calls, at least 60 variant calls, at least 70 variant calls, at least 80 variant calls, at least 90 variant calls, at least 100 variant calls, at least 200 variant calls, at least 300 variant calls, at least 400 variant calls, at least 500 variant calls, at least 600 variant calls, at least 700 variant calls, at least 800 variant calls, at least 900 variant calls, at least 1000 variant calls, at least 2000 variant calls, at least 3000 variant calls, at least 4000 variant calls, between 10 and 10,000 variant calls, between 50 and 5000 variant calls or between 100 and 4500 variant calls for the test subject using the sequencing data obtained from the biological sample of the test subject. In some embodiments, the systems and methods of the present disclosure compute the plurality of variant calls within one day, within one hour, within thirty minutes, within 15 minutes, within 5 minutes, or within on minute of obtaining the methylation sequencing data of the test subject.

In some embodiments, with reference to block 348 and/or block 350, the method further comprises obtaining a second plurality of variant calls using a second plurality of nucleic acid fragment sequences, in electronic form, acquired from a second plurality of nucleic acid fragments in a second biological sample of the test subject by whole genome sequencing, where the second plurality of nucleic acid fragments are cell-free nucleic acid fragments and where the second biological sample is a matched germline sample from the subject (e.g., a liquid biological sample such as whole blood), and removing each respective variant call from the plurality of variant calls that is also in the second plurality of variant calls (e.g., removing germline variant calls). This is further described in blocks 304 and 306 above.

In some embodiments, the method further comprises removing a respective variant call from the plurality of variant calls that is in a list of known germline variants as described in block 308 above. In some embodiments, the method further comprises removing a respective variant call from the plurality of variant calls when the respective variant call is found in a tissue sample of a subject other than the test subject as discussed in further detail in block 310 above.

In some embodiments, the method further comprises removing a respective variant call from the plurality of variant calls when the respective variant call fails to satisfy a quality metric as discussed in block 312 above. In some embodiments, the quality metric is a minimum variant allele fraction in the first plurality of nucleic acid fragment sequences, in electronic form, that map to the allelic position of the respective variant call. In some embodiments, the minimum variant allele fraction is ten percent. In some embodiments, the minimum variant allele fraction is less than 1 percent, less than 2 percent, less than 3 percent, less than 4 percent, less than 5 percent, less than 6 percent, less than 7 percent, less than 8 percent, less than 9 percent, less than 10 percent less than 15 percent, or less than 20 percent.

In some embodiments, the quality metric is a maximum variant allele fraction in the first plurality of nucleic acid fragment sequences, in electronic form, that map to the allelic position of the respective variant call. In some embodiments, the maximum variant allele fraction is ninety percent. In some embodiments, the maximum variant allele fraction is at least 55 percent, at least 60 percent, at least 70 percent, at least 80 percent, at least 90 percent, at least 95 percent, or at least 99 percent.

In some embodiments, the quality metric is a minimum depth in the first plurality of nucleic acid fragment sequences, in electronic form, that map to the allelic position of the respective variant call. In some embodiments, the minimum depth is ten. In some embodiments, the minimum depth is at least 5, at least 10, at least 50, at least 100, or at least 200.

In some embodiments, with reference to block 348 and/or block 350, in some embodiment the plurality of variant calls is filtered by one or more filters. In some embodiments, the filtering occurs prior to the determination of the plurality of variant calls for the test subject. In some embodiments, the filtering occurs after the method determines the plurality of variant calls for the test subject (e.g., thus resulting in a secondary, reduced plurality of variant calls that are reported to the test subject or that are used for tumor fraction determination).

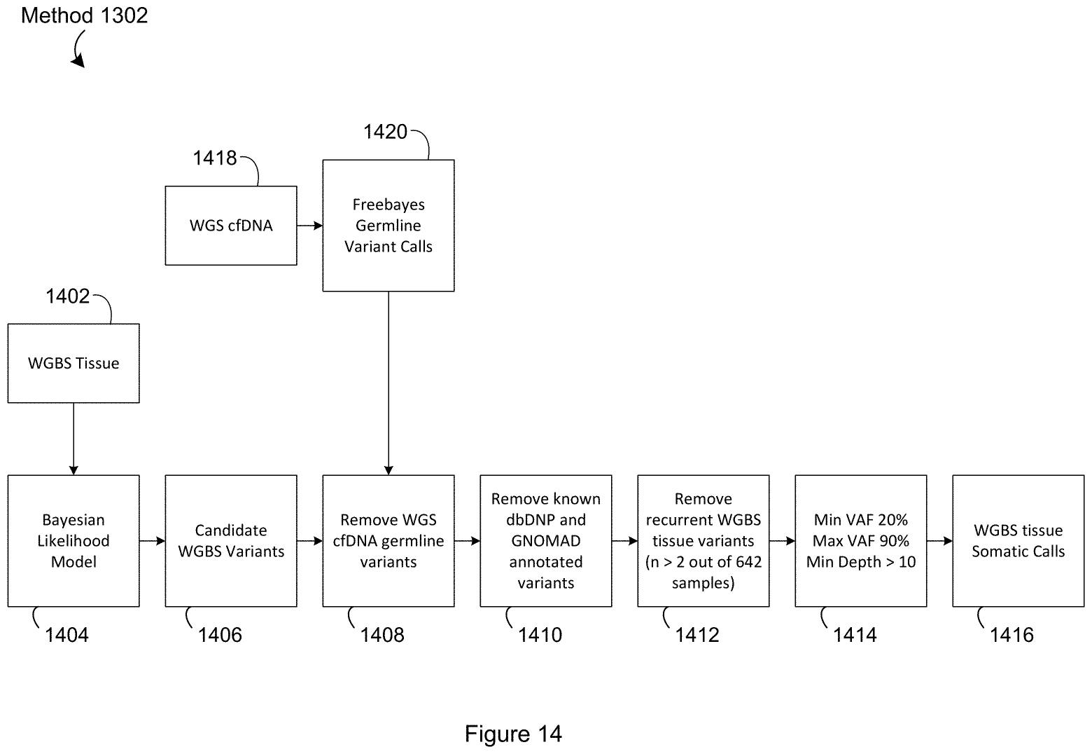

In some embodiments, the one or more filters are selected from the set comprising a minimum variant allele frequency (e.g. 1434 of FIG. 14), a maximum variant allele frequency (e.g., 1436 of FIG. 14B), a minimum sequencing depth for a respective allele (e.g., 1438 of FIG. 14B), a blacklist of germline variants from the test subject (e.g., as marked by freebayes) and further described in block 306 (e.g., block 1446), a blacklist of a custom database (e.g., the recurrent tissue blacklist 310 of FIG. 3A, and block 1444 of FIG. 14), or a blacklist of germline variants from a reference database (e.g., from the gnomad and/or dbSNP databases, blocks 1440 and 1442 of FIG. 14B and further described above with reference to block 308).

With reference to block 1432 of FIG. 14B, in some embodiments each variant allele that is identified using systems and methods described in conjunction with FIGS. 3B through 3D, in order to be retained for further use in a pipeline (e.g., to determine tumor fraction), must be supported by at least one nucleic acid fragment that has the variant allele. In other words, the sequence reads from the test subject must include sequencing information for at least one nucleic acid fragment from the test subject that maps to the genomic region of the variant allele. In alternative embodiments, the sequence reads from the test subject must include sequencing information for at least 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 200, 300, 400, 500, or 1000 different nucleic acid fragments from the test subject that map to the genomic region of the variant allele and have the variant allele in order for the variant allele to be retained for further use in a pipeline.

With reference to block 1434 of FIG. 14B, in some embodiments, each variant allele that is identified using systems and methods described in conjunction with FIGS. 3B through 3D, in order to be retained for further use in a pipeline (e.g., to determine tumor fraction), must have a minimum variant allele frequency (minimum VAF) of 20%. That is, the variant allele must occur in at least 20% of the nucleic acid fragments from the test subject. In alternative embodiments, the minimum allele frequency is at least 3%, at least 5%, at least 10%, at least 15%, at least 25%, at least 30%, at least 35%, at least 40%, at least 45%, or at least 50% of the nucleic acid fragments from the test subject.

With reference to block 1436 of FIG. 14B, in some embodiments, each variant allele that is identified using the systems and methods described in conjunction with FIGS. 3B through 3D, in order to be retained for further use in a pipeline (e.g., to determine tumor fraction), must have a maximum variant allele frequency (maximum VAF) of 90%. That is, the variant allele must occur in no more than 90% of the nucleic acid fragments from the test subject. In alternative embodiments, the maximum allele frequency 95% or less, 85% or less, 80% or less, 75% or less, 70% or less, 65% or less, 60% or less, 55% or less, or 50% or less of the nucleic acid fragments from the test subject.

With reference to block 1438 of FIG. 14B, in some embodiments, each variant allele that is identified using the systems and methods described in conjunction with FIGS. 3B through 3D, in order to be retained for further use in a pipeline (e.g., to determine tumor fraction), must be supported by an overall sequencing depth of at least 10. In other words, the sequence reads from the test subject must include sequencing information for at least 10 different nucleic acid fragments from the test subject that map to the genomic region of the variant allele. The filter of block 1438 does not require that each of these fragments have the variant allele. Rather, the filter of block 1438 is a sequencing depth requirement. In alternative embodiments, the sequence reads from the test subject must include sequencing information for at least 15, 20, 25, 30, 35, 40, 45, 50, 55, 60, 65, 70, 75, 80, 85, 90, 95, 100, 200, 300, 400, 500, or 1000 nucleic acid fragments from the test subject that map to the genomic region of the variant allele in order for the variant allele to be retained for further use in a pipeline.

With reference to block 1440 of FIG. 14B, in some embodiments, each variant allele that is identified using the systems and methods described in conjunction with FIGS. 3B through 3D, in order to be retained for further use in a pipeline (e.g., to determine tumor fraction), must not be present in a list of generally known germline variants, such as the dbSNP dataset. See Karczewski et al., 2019, “Variation across 141,456 human exomes and genomes reveals the spectrum of loss-of-function intolerance across human protein-coding genes,” bioRxiv doi.org/10.1101/531210 and Sherry et al., 2011, “dbSNP: the NCBI database of genetic variation” Nuc. Acids. Res. 29, 308-311, respectively.

With reference to block 1442 of FIG. 14B, in some embodiments, each variant allele that is identified using the systems and methods described in conjunction with FIGS. 3B through 3D, in order to be retained for further use in a pipeline (e.g., to determine tumor fraction), must not be present in a list of generally known germline variants, such as the gnomAD dataset. See Karczewski et al., 2019, “Variation across 141,456 human exomes and genomes reveals the spectrum of loss-of-function intolerance across human protein-coding genes,” bioRxiv doi.org/10.1101/531210 and Sherry et al., 2011, “dbSNP: the NCBI database of genetic variation” Nuc. Acids. Res. 29, 308-311, respectively.

With reference to block 1444 of FIG. 14B, in some embodiments, each variant allele that is identified using the systems and methods described in conjunction with FIGS. 3B through 3D, in order to be retained for further use in a pipeline (e.g., to determine tumor fraction), must not reside in a blacklist of known noisy genomic positions. In some embodiments such sites is based on a set of 642 samples from the CCGA Approach 1 method described above in Example 5). In some embodiments, the blacklist is all or a portion of the ENCODE blacklist. See Ameniya et al. 2019, “The ENCODE Blacklist: Identification of Problematic Regions of the Genome,” Scientific Reports 9, article number 9354.

With reference to block 1446 of FIG. 14B, in some embodiments, each variant allele that is identified using the systems and methods described in conjunction with FIGS. 3B through 3D, in order to be retained for further use in a pipeline (e.g., to determine tumor fraction), must not be identified as a germline variant. In some embodiments, a variant allele is identified as a germline variant when a variant caller algorithm, such as: FreeBayes, VarDict, MuTect, MuTect2, MuSE, FreeBayes, VarDict, and/or MuTect (see Bian, 2018, “Comparing the performance of selected variant callers using synthetic data and genome segmentation,” BMC Bioinformatics 19:429, which is hereby incorporated by reference) identifies the variant as a germline variant, private to a test subject within sample-matched WGS cfDNA.

Block 1448 of FIG. 14B shows the performance gain when the filter described above in conjunction with block is 346 is applied. Referring to block 346 of FIG. 3C, in some embodiments, the systems and methods of the present disclosure determine whether any of a plurality of likelihoods supports a variant call at the allelic position. In some embodiments, this comprises determining whether any likelihood in the plurality of likelihoods for any of the proposed genotypes for the allelic position satisfies a variant threshold. In some embodiments, when a likelihood for any of the proposed genotypes for the allelic position satisfies a variant threshold, a variant at the allelic position is called. In such embodiments, when a likelihood for any of the proposed genotypes for the allelic position does not satisfy a variant threshold, a variant at the allelic position is not called.

In some embodiments, two or more of the filters illustrated in FIG. 14B and discussed above are used to filter the plurality of variant calls.

In some embodiments, when two or more filters are used, the ordering of the two or more filters is predetermined.

In some embodiments, when two or more filters are used, there is no particular requirement on the order of the filters used. For instance, in some embodiments, there is no requirement that the filters be applied in the order illustrated in FIG. 14B or, in fact be in any particular order.

In some embodiments, all of the filters in the set comprising a minimum variant allele frequency, a maximum variant allele frequency, a minimum depth at the allele, a blacklist of germline variants from the test subject, a blacklist of a custom database, or a blacklist of germline variants from a reference database are used to filter the plurality of variant calls. In some embodiments, the plurality of filters illustrated in FIG. 14B and described in Example 7 are used to filter the plurality of variant calls. In some embodiments, one or more additional filters are used in filtering the plurality of variant calls.

White Blood Cell Clonal Expansion.

In some embodiments, the systems and methods of the present disclosure comprise using the plurality of variant calls, optionally after application of any combination of the filters described in the present disclosure, to quantify white blood cell clonal expansion (the expansion of a clonal population of blood cells with one or more somatic mutations). That is, the systems and methods of the present disclosure provide reliable methods for calling somatic SNPs as well as germ line SNPs. As such, this variant allele data can be used to ascertain clonal expansion/clinical hematopoiesis. For instance Sano, 2018, “Clonal Hematopoiesis and its Impact on Cardiovascular Disease, Circle J. 83(1), 2-11, Natarajan et al., “Clinal Hematopoiesis Somatic Mutations in Blood cells and Atherosclerosis,” Genomic and Precision Medicine 11(7); and Tajddin et al, 2016, “Large-Scale Exome-wide Association Analysis Identifies Loci for White Blood Cell Traits and Pleiotropy with Immune-Mediated Diseases,” Am J. Humn Gent 99(1), 22-39 disclose loci and alternate alleles that are associated with white blood cell clonal expansion. Such loci can be evaluated using the systems of the methods of the present disclosure to ascertain clonal expansion associated with specific diseases and/or the risk of clonal expansion associated with certain diseases.

Tumor Fraction Estimation.

In some embodiments, the systems and methods of the present disclosure further comprise using the plurality of variant calls that were discovered using any of the methods described in FIGS. 3B through 3D, optionally after the application of any combination of filters discussed in FIG. 3A and/or FIG. 14 and/or FIG. 15, to perform tumor fraction estimation. In some such embodiments, such tumor fraction estimates are used to detect cancer in the subject.

In some embodiments, the systems and methods of the present disclosure comprise using the plurality of variant calls to assess a genetic risk (e.g., a risk of carrying or of expressing a heritable disease) of the subject through germline analysis using the plurality of variant calls. In some embodiments, for example, if the biological sample for a respective reference subject is derived from cell-free nucleic acids, the cell-free nucleic acids may exhibit an appreciable tumor fraction. In some embodiments, the corresponding tumor fraction, with respect to the respective reference subject is at least two percent, at least five percent, at least ten percent, at least fifteen percent, at least twenty percent, at least twenty-five percent, at least fifty percent, at least seventy-five percent, at least ninety percent, at least ninety-five percent, or at least ninety-eight percent.

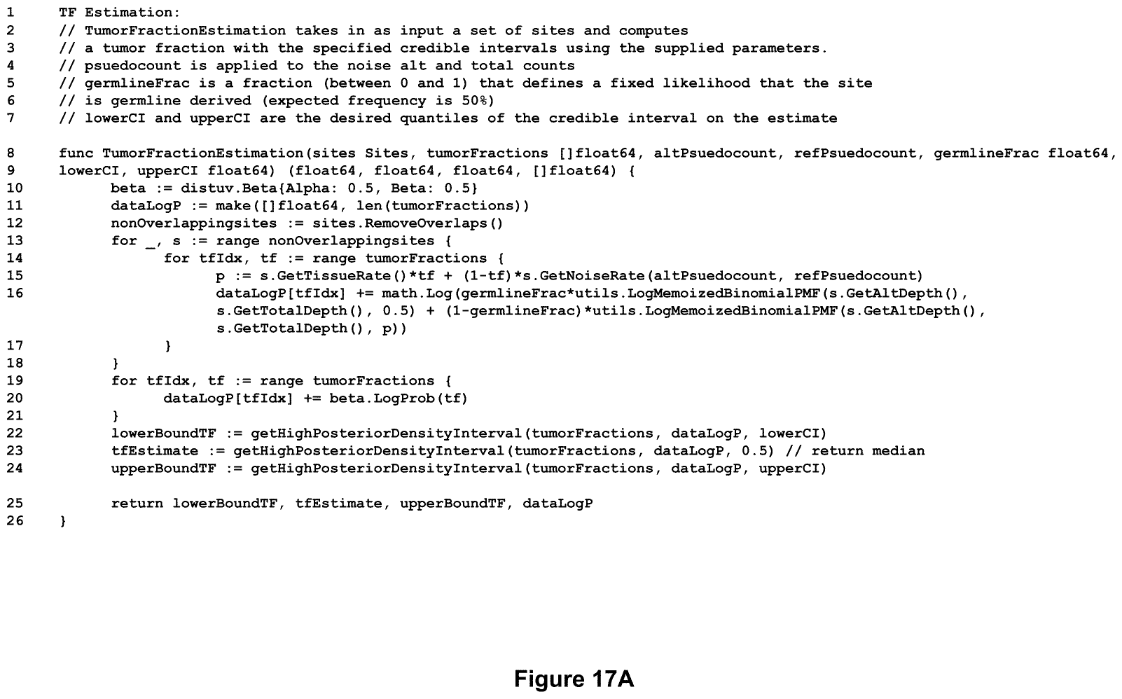

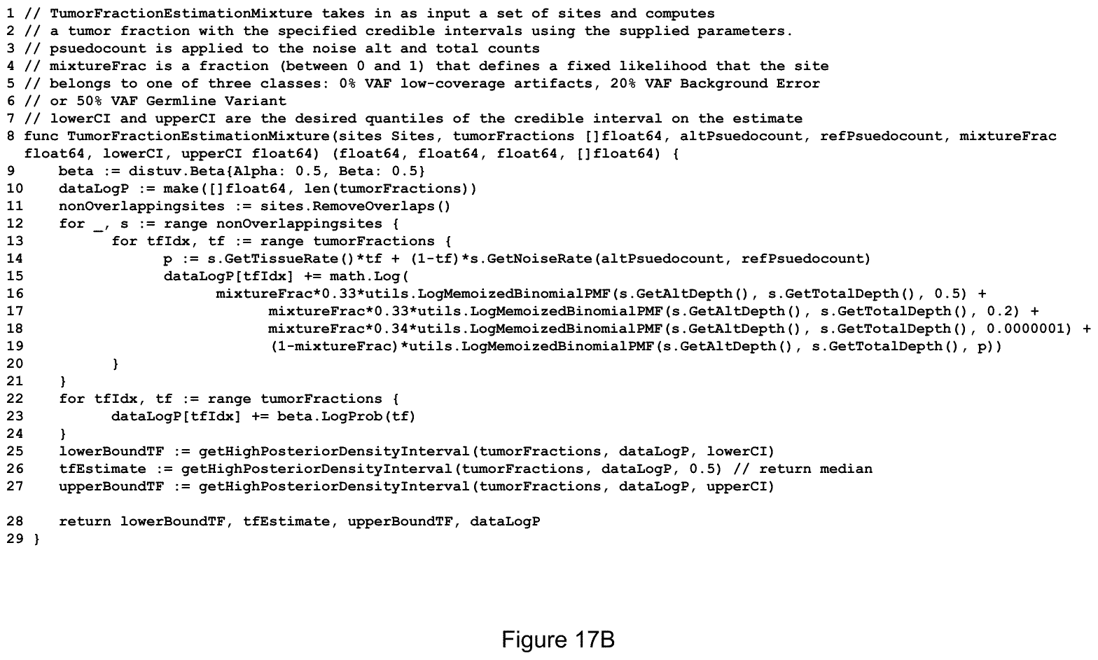

In some embodiments, the corresponding tumor fraction, with respect to the test subject, is determined using counts of fragments supporting and not supporting each variant that were generated from WGS sequencing of corresponding cfDNA samples matched to the WGBS data (e.g., the calls for each allele in the plurality of allelic positions from block 1448 of FIG. 15, block 1416 of FIG. 14, or block 348 of FIG. 3D). In some such embodiments, posterior tumor fraction estimates are calculated using a grid search over tumor fraction candidates and a per-variant likelihood defined as a mixture of binomial likelihoods is employed. The mixture components accounted for (1) observing fragments due to tumor shedding as well as (2) various error modes including germline variants and falsely called variants. Median and 95% credible intervals were calculated for each participant's tumor fraction. In this vain, FIGS. 17A and 17B illustrate two different methods for determining a tumor fraction estimate using the variant allele calls for the plurality of allelic positions from block 1448 of FIG. 15, block 1416 of FIG. 14, or block 348 of FIG. 3D. Lines 1-7 of FIG. 17A are comments that explain that the program illustrated in FIG. 17A is directed to taking as input a set of sites (e.g., plurality of allelic positions from block 1448 of FIG. 15, block 1416 of FIG. 14, or block 348 of FIG. 3D) and computing from them a tumor fraction within specified credible intervals (lower CI to upper CI) using the supplied parameters. The program makes an assumption on the germline fraction of the sample (germlineFrac) which is a fraction (between 0 and 1) that defines a fixed likelihood that any given allelic position (site) is germline derived. In FIG. 17A, this expected frequency is set to 50% but it can be changed to any value between zero and 100% in alternative embodiments. lowerCI and upperCI are the desired quantiles of the credible interval on the estimate. The lower bound (lowerboundTF) is a value less than the upper bound (upperBountTF), where both lowerboundTF and upperBountTF are each a different value between zero and 100 percent.

Lines 1-7 of FIG. 17B are comments that explain that the program illustrated in FIG. 17B is directed to taking as input a set of sites (e.g., the calls for each allele in the plurality of allelic positions from block 1448 of FIG. 15, block 1416 of FIG. 14, or block 348 of FIG. 3D) and computing from them a tumor fraction within specified credible intervals (lower CI to upper CI) using supplied parameters. The program makes an assumption on the mixture fraction of the sample (mixtureFrac), which is a fraction (between 0 and 1) that defines a fixed likelihood that any given allelic position (site) belongs to one of three classes 0% variant-allele frequency low-coverage artifacts, 20% variant allele background error, and 50% variant allele frequency germline variant. In some embodiments, the probabilities for these three classes are adjusted to different values between zero percent and 100 percent. In the program of FIG. 17B, lowerCI and upperCI are the desired quantiles of the credible interval on the tumor fraction estimate. The lower bound (lowerboundTF) is a value less than the upper bound (upperBountTF), where both lowerboundTF and upperBountTF are each a different value between zero and 100 percent.

Recurring Basis.

In some embodiments, the tumor fraction or clonal expansion assessment is determined on a recurring basis over time for minimal residual disease and recurrence monitoring. In some such embodiments, the determination of tumor fraction (or clonal expansion) is performed from a first sample obtained before and a second sample obtained after a cancer treatment to assess the efficacy of the cancer treatment.

In some embodiments, the method repeating the estimating the tumor fraction estimate (or clonal expansion estimate) for a test subject at each respective time point in a plurality of time points across an epoch, thus obtaining a corresponding tumor fraction estimate (or clonal expansion estimate), in a plurality of tumor fraction estimates (or clonal expansion estimate), for the test subject at each respective time point. In some embodiments this plurality of tumor fraction estimates (or clonal expansion estimates) is used to determine a state or progression of a disease condition in the test subject during the epoch in the form of an increase or decrease of tumor fraction (or clonal expansion) over the epoch.

In some embodiments, each epoch is a period of months and each time point in the plurality of time points is a different time point in the period of months. In some embodiments, the period of months is less than four months. In some embodiments, each epoch is one month long. In some embodiments, each epoch is two months long. In some embodiments, each epoch is three months long. In some embodiments, each epoch is four months long. In some embodiments, each epoch is five, six, seven, eight, nine, ten, eleven, twelve, thirteen, fourteen, fifteen, sixteen, seventeen, eighteen, nineteen, twenty, twenty-one, twenty-two, twenty-three or twenty-four months long.

In some embodiments, the epoch is a period of years and each time point in the plurality of time points is a different time point in the period of years. In some embodiments, the period of years is between one year and ten years. In some embodiments, the period of years is one year, two years, three years, four years, five years, six years, seven years, eight years, nine years, or ten years. In some embodiment the epoch is between one and thirty years.

In some embodiments, the epoch is a period of hours and each time point in the plurality of time points is a different time point in the period of hours. In some embodiments, the period of hours is between one hour and twenty-four hours. In some embodiments, the period of hours is 1, 2, 3, 4, 5, 6, 7, 8, 9, 10, 11, 12, 13, 14, 15, 16, 17, 18, 19, 20, 21, 22, 23, or 24 hours.

In some embodiments, a diagnosis of the test subject is changed when the tumor fraction estimate (or clonal expansion estimate) of the subject is observed to change by a threshold amount across the epoch. For instance, in some embodiments, the diagnosis is changed from having cancer to being in remission. As another example, in some embodiments, the diagnosis is changed from not having cancer to having cancer. As another example, in some embodiments, the diagnosis is changed from having a first stage of a cancer to having a second stage of a cancer. As another example, in some embodiments, the diagnosis is changed from having a second stage of a cancer to having a third stage of a cancer. As still another example, in some embodiments, the diagnosis is changed from having a third stage of a cancer to having a fourth stage of a cancer. As still another example, in some embodiments, the diagnosis is changed from having a cancer that has not metastasized to having a cancer that has metastasized.

In some embodiments, a prognosis of the test subject is changed when the tumor fraction estimate (or clonal expansion estimate) of the subject is observed to change by a threshold amount across the epoch. For example, in some embodiments, the prognosis involves life expectancy and the prognosis is changed from a first life expectancy to a second life expectancy, where the first and second life expectancy differ in their duration. In some embodiments, the change in prognosis increases the life expectancy of the subject. In some embodiments, the change in prognosis decreases the life expectancy of the subject.

In some embodiments, a treatment of the test subject is changed when the tumor fraction estimate (or clonal expansion estimate) of the subject is observed to change by a threshold amount across the epoch. In some embodiments, the changing of the treatment comprises initiating a cancer medication, increasing the dosage of a cancer medication, stopping a cancer medication, or decreasing the dosage of the cancer medication. In some embodiments, the changing of the treatment comprises initiating or terminating treatment of the subject with Lenalidomid, Pembrolizumab, Trastuzumab, Bevacizumab, Rituximab, Ibrutinib, Human Papillomavirus Quadrivalent (Types 6, 11, 16, and 18) Vaccine, Pertuzumab, Pemetrexed, Nilotinib, Nilotinib, Denosumab, Abiraterone acetate, Promacta, Imatinib, Everolimus, Palbociclib, Erlotinib, Bortezomib, Bortezomib, or a generic equivalent thereof. In some embodiments, the changing of the treatment comprises increasing or decreasing a dosage of Lenalidomid, Pembrolizumab, Trastuzumab, Bevacizumab, Rituximab, Ibrutinib, Human Papillomavirus Quadrivalent (Types 6, 11, 16, and 18) Vaccine, Pertuzumab, Pemetrexed, Nilotinib, Nilotinib, Denosumab, Abiraterone acetate, Promacta, Imatinib, Everolimus, Palbociclib, Erlotinib, Bortezomib, Bortezomib, or a generic equivalent thereof administered to the subject. In some embodiments, the threshold is greater than ten percent, greater than twenty percent, greater than thirty percent, greater than forty percent, greater than fifty percent, greater than two-fold, greater than three-fold, or greater than five-fold.

In some embodiments, the tumor fraction estimate for the test subject is between 0.003 and 1.0. In some embodiments, the tumor fraction estimate for the test subject is between 0.005 and 0.80. In some embodiments, the tumor fraction estimate for the test subject is between 0.01 and 0.70. In some embodiments, the tumor fraction estimate for the test subject is between 0.05 and 0.60.

In some embodiments, a treatment regimen is applied to the test subject based, at least in part, on a value of the tumor fraction estimate (or clonal expansion estimate) for the test subject. In some embodiments, the treatment regimen comprises applying an agent for cancer to the test subject. In some embodiments, the agent for cancer is a hormone, an immune therapy, radiography, or a cancer drug. In some embodiments, the agent for cancer is Lenalidomid, Pembrolizumab, Trastuzumab, Bevacizumab, Rituximab, Ibrutinib, Human Papillomavirus Quadrivalent (Types 6, 11, 16, and 18) Vaccine, Pertuzumab, Pemetrexed, Nilotinib, Nilotinib, Denosumab, Abiraterone acetate, Promacta, Imatinib, Everolimus, Palbociclib, Erlotinib, Bortezomib, Bortezomib, or a generic equivalent thereof.

In some embodiments, the test subject has been treated with an agent for cancer and the tumor fraction estimate for the test subject is used to evaluate a response of the subject to the agent for cancer. In some embodiments, the agent for cancer is a hormone, an immune therapy, radiography, or a cancer drug. In some embodiments, the agent for cancer is Lenalidomid, Pembrolizumab, Trastuzumab, Bevacizumab, Rituximab, Ibrutinib, Human Papillomavirus Quadrivalent (Types 6, 11, 16, and 18) Vaccine, Pertuzumab, Pemetrexed, Nilotinib, Nilotinib, Denosumab, Abiraterone acetate, Promacta, Imatinib, Everolimus, Palbociclib, Erlotinib, Bortezomib, Bortezomib, or a generic equivalent thereof.

In some embodiments, the test subject has been treated with an agent for cancer and the tumor fraction estimate for the test subject is used to determine whether to intensify or discontinue the agent for cancer in the test subject. For instance, in some embodiments, observation of at least a tumor fraction estimate (e.g., greater than 0.05, 0.10, 0.15, 0.20, 0.25, or 0.30, etc.) is used as a basis for intensifying (e.g., increasing the dosage, increasing radiation level in radiation treatment) of the agent for cancer in the test subject. In some embodiments, observation of less than a threshold tumor fraction estimate (e.g., less than 0.05, 0.10, 0.15, 0.20, 0.25, or 0.30, etc.) is used as a basis for discontinuing use of the agent for cancer in the test subject.

In some embodiments, the test subject has been subjected to a surgical intervention to address the cancer and the tumor fraction estimate for the test subject is used to evaluate a condition of the test subject in response to the surgical intervention. In some embodiments the condition is a metric based upon the tumor fraction estimate using the methods provided in the present disclosure.

Detect Contamination.

In some embodiments, the systems and methods of the present disclosure comprise using the plurality of variant calls, optionally after application of one or more of the filters described in the present disclosure, to detect contamination using SNPs. For instance, in some embodiments the plurality of variant calls, optionally after application of one or more of the filters described in the present disclosure are used to detecting cross-contamination using the techniques disclosed in U.S. patent application Ser. No. 15/900,645, entitled “Detecting cross-contamination in sequencing data using regression techniques,” filed Feb. 20, 2018 and published as US 2018/0237838, U.S. patent application Ser. No. 16/019,315, entitled “Detecting cross-contamination in sequencing data,” filed Jun. 26, 2018 and published as US 2018/0373832, and/or U.S. Application No. 63/080,670, entitled “Detecting cross-contamination in sequencing data,” filed Sep. 18, 2020.

### Additional Embodiments

### Example 1—Difficulties of Identifying Somatic Variants

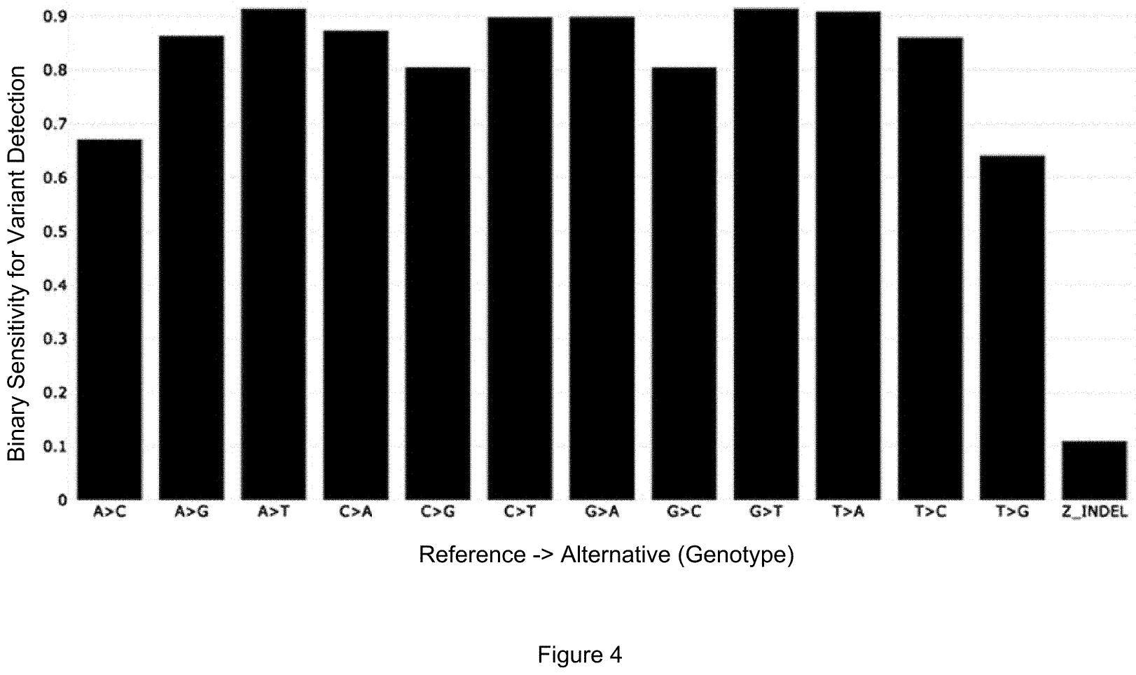

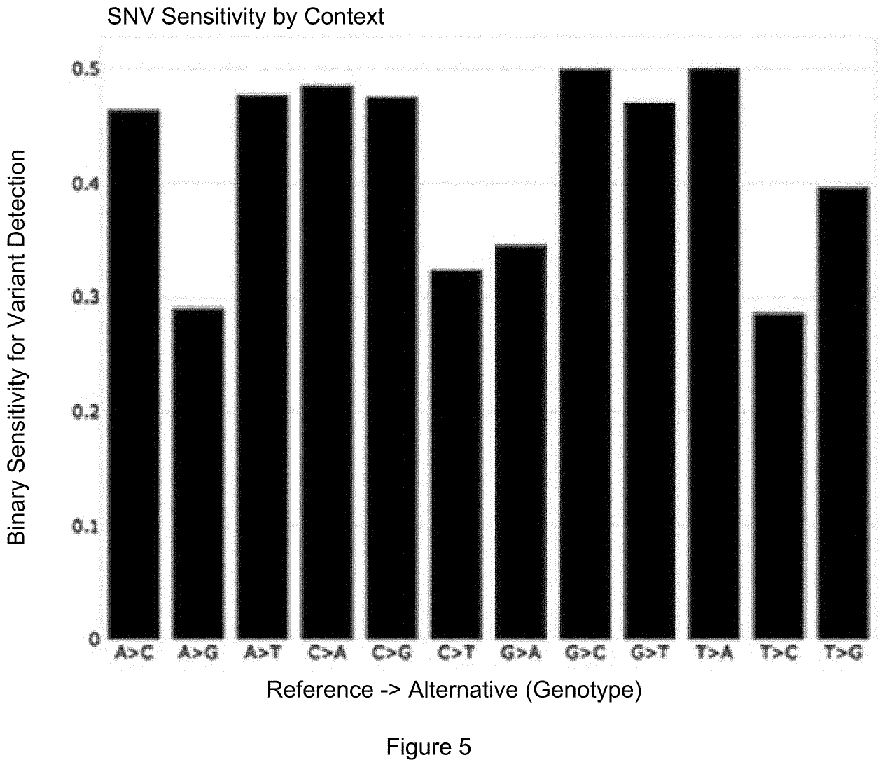

Given a single biological sample, it can be difficult to distinguish between germline and somatic variants. Since somatic variants are more closely connected with the development of cancer this impacts the ability of healthcare providers to determine appropriate treatment recommendations for patients. As seen in FIG. 4, over 60% of germline variants can be identified from bisulfite-treated biological samples, excluding indel variants. Both WGS and WGBS sequencing (e.g., as described with reference to FIG. 3A) were used to call the variants shown in FIG. 4. As further illustrated in FIG. 5, the ability to detect variants decreases when only single strand support is available.

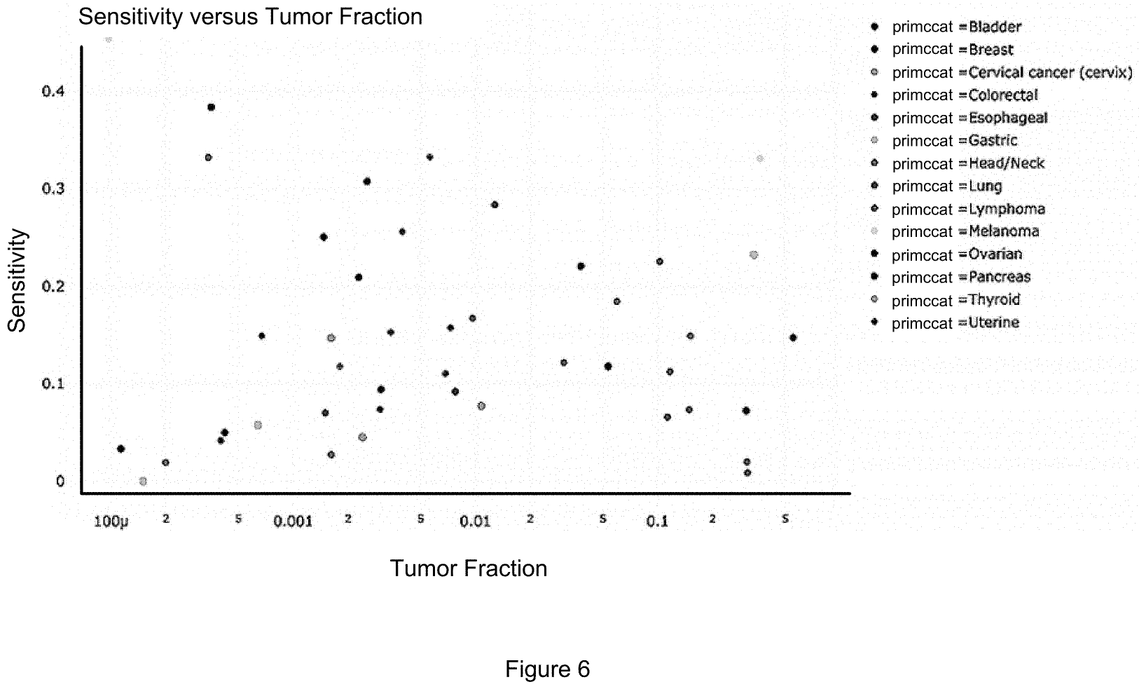

The detection rate of somatic variants is much lower. FIG. 6 provides an example. In FIG. 6, 44 paired WGBS and WGS cfDNA human samples were analyzed for variants on chromosome 1. The overall sensitivity for determining somatic variants using previously known methods was only 15%, regardless of known tumor fraction of the samples. Such a low percentage does not enable accurate detection of somatic variants, and improved detection methods are required.

An analysis of WGS data alone using multiple variant identification methods (e.g., including dbSNP and gnomad) revealed an aggregate sensitivity rate of 15.35% that is similar to, or slightly higher than, the sensitivity rate from the combination of WGS and WGBS data, as exemplified by FIG. 6. In particular, WGS analysis identified 12,124 true positive and 7,750 false-positive variants, out of a total number of 78,975 somatic variants.

In light of the issues highlighted here for identifying somatic variants, new methods are needed in the art.

### Example 2—Obtaining a Plurality of Sequence Reads

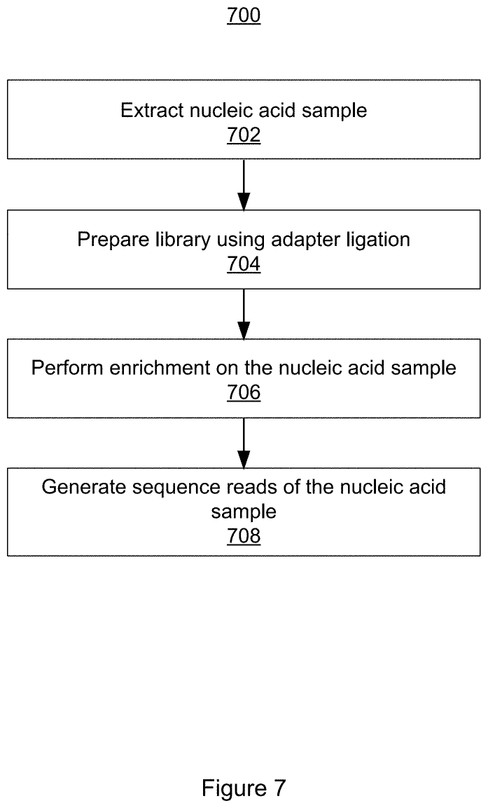

FIG. 7 is a flowchart of method 700 for preparing a nucleic acid sample for sequencing according to some embodiments of the present disclosure. The method 700 includes, but is not limited to, the following steps. For example, any step of method 700 may comprise a quantitation sub-step for quality control or other laboratory assay procedures known to one skilled in the art.

In block 702, a nucleic acid sample (DNA or RNA) is extracted from a subject. The sample may be any subset of the human genome, including the whole genome. The sample may be extracted from a subject known to have or suspected of having cancer. The sample may include blood, plasma, serum, urine, fecal, saliva, other types of bodily fluids, or any combination thereof. In some embodiments, methods for drawing a blood sample (e.g., syringe or finger prick) may be less invasive than procedures for obtaining a tissue biopsy, which may require surgery. The extracted sample may comprise cfDNA and/or ctDNA. For healthy individuals, the human body may naturally clear out cfDNA and other cellular debris. If a subject has a cancer or disease, ctDNA in an extracted sample may be present at a detectable level for diagnosis.

In block 704, a sequencing library is prepared. During library preparation, unique molecular identifiers (UMI) are added to the nucleic acid molecules (e.g., DNA molecules) through adapter ligation. The UMIs are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. During PCR amplification following adapter ligation, the UMIs are replicated along with the attached DNA fragment. This provides a way to identify sequence reads that came from the same original fragment in downstream analysis.

In block 706, targeted DNA sequences are enriched from the library. During enrichment, hybridization probes (also referred to herein as “probes”) are used to target, and pull down, nucleic acid fragments informative for the presence or absence of cancer (or disease), cancer status, or a cancer classification (e.g., cancer class or tissue of origin). For a given workflow, the probes may be designed to anneal (or hybridize) to a target (complementary) strand of DNA. In some embodiments each probe is between 8 and 5000 bases in length, between 12 and 2500 bases in length, or between 15 and 1225 bases in length. The target strand may be the “positive” strand (e.g., the strand transcribed into mRNA, and subsequently translated into a protein) or the complementary “negative” strand. In some embodiments the probes may range in length from tens, hundreds or thousands of base pairs.

In some embodiments, the probes are designed based on a methylation site panel.

In some embodiments, the probes are designed based on a panel of targeted genes and/or genomic regions to analyze particular mutations or target regions of the genome (e.g., of the human or another organism) that are suspected to correspond to certain cancers or other types of diseases. For instance in some embodiments, each of the probes uniquely maps to a genomic region described in International Patent Publication Nos. WO2020154682A3, WO2020/069350A1, or WO2019/195268A2, each of which is hereby incorporated by reference.

In some embodiments, the probes cover overlapping portions of a target region. With reference to block 708, in some embodiments the probes are used to generate sequence reads of the nucleic acid sample.

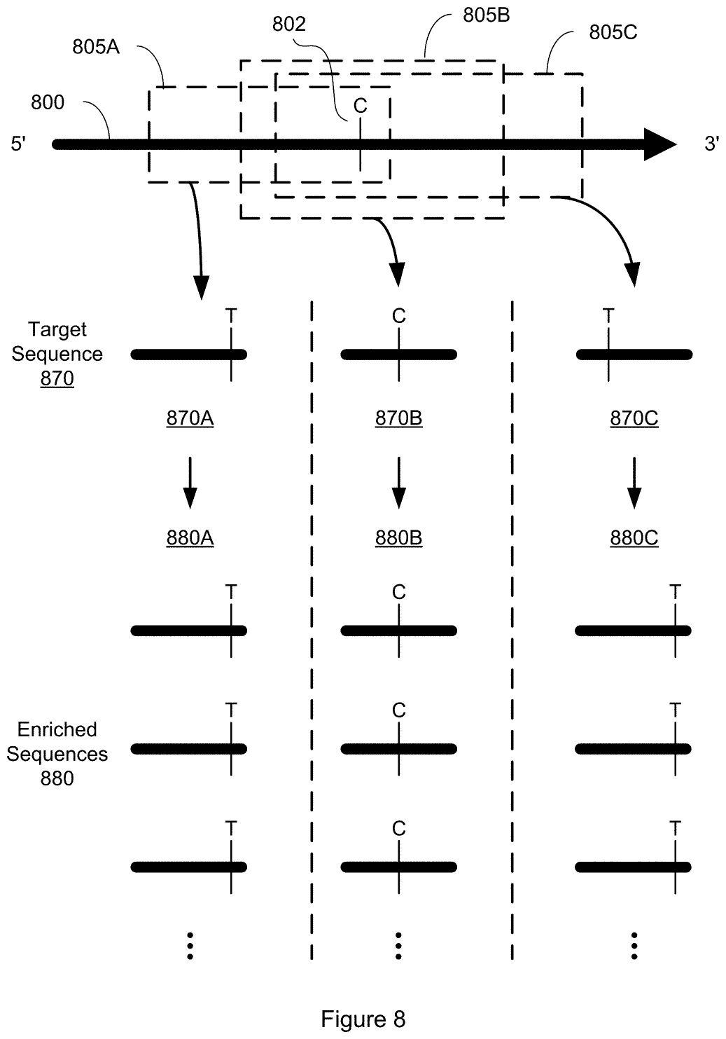

FIG. 8 is a graphical representation of the process for obtaining sequence reads according to one embodiment. FIG. 8 depicts one example of a nucleic acid segment 800 from the sample. Here, the nucleic acid segment 800 can be a single-stranded nucleic acid segment. In some embodiments, the nucleic acid segment 800 is a double-stranded cfDNA segment. The illustrated example depicts three regions 805A, 805B, and 805C of the nucleic acid segment that can be targeted by different probes. Specifically, each of the three regions 805A, 805B, and 805C includes an overlapping position on the nucleic acid segment 800. An example overlapping position is depicted in FIG. 8 as the cytosine (“C”) nucleotide base 802. The cytosine nucleotide base 802 is located near a first edge of region 805A, at the center of region 805B, and near a second edge of region 805C.

In some embodiments, one or more (or all) of the probes are designed based on a gene panel or methylation site panel to analyze particular mutations or target regions of the genome (e.g., of the human or another organism) that are suspected to correspond to certain cancers or other types of diseases. By using a targeted gene panel or methylation site panel rather than sequencing all expressed genes of a genome, also known as “whole-exome sequencing,” the method 800 may be used to increase sequencing depth of the target regions, where depth refers to the count of the number of times a given target sequence within the sample has been sequenced. Increasing sequencing depth reduces required input amounts of the nucleic acid sample. For instance, in some embodiments, a targeted gene panel or methylation site panel comprises a plurality of probes where each of the probes uniquely maps to a genomic region described in International Patent Publication Nos. WO2020154682A3, WO2020/069350A1, or WO2019/195268A2, each of which is hereby incorporated by reference.

Hybridization of the nucleic acid sample 800 using one or more probes results in an understanding of a target sequence 870. As shown in FIG. 8, the target sequence 870 is the nucleotide base sequence of the region 805 that is targeted by a hybridization probe. The target sequence 870 can also be referred to as a hybridized nucleic acid fragment. For example, target sequence 870A corresponds to region 805A targeted by a first hybridization probe, target sequence 870B corresponds to region 805B targeted by a second hybridization probe, and target sequence 870C corresponds to region 805C targeted by a third hybridization probe. Given that the cytosine nucleotide base 802 is located at different locations within each region 805A-C targeted by a hybridization probe, each target sequence 870 includes a nucleotide base that corresponds to the cytosine nucleotide base 802 at a particular location on the target sequence 870.

After a hybridization step, the hybridized nucleic acid fragments are captured and may also be amplified using PCR. For example, the target sequences 870 can be enriched to obtain enriched sequences 880 that can be subsequently sequenced. In some embodiments, each enriched sequence 880 is replicated from a target sequence 870. Enriched sequences 880A and 880C that are amplified from target sequences 870A and 870C, respectively, also include the thymine nucleotide base located near the edge of each sequence read 880A or 880C. As used hereafter, the mutated nucleotide base (e.g., thymine nucleotide base) in the enriched sequence 880 that is mutated in relation to the reference allele (e.g., cytosine nucleotide base 802) is considered as the alternative allele. Additionally, each enriched sequence 880B amplified from target sequence 870B includes the cytosine nucleotide base located near or at the center of each enriched sequence 880B.

In block 708 of FIG. 7, sequence reads are generated from the enriched DNA sequences, e.g., enriched sequences 880 shown in FIG. 8. Sequencing data may be acquired from the enriched DNA sequences by known means in the art. For example, the method 800 may include next-generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators.

In some embodiments, the sequence reads may be aligned to a reference genome using known methods in the art to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene.

In some embodiments, an average sequence read length of a corresponding plurality of sequence reads obtained by the methylation sequencing for a respective fragment is between 140 and 280 nucleotides.

In various embodiments, a sequence read is comprised of a read pair denoted as R₁ and R₂. For example, the first read R₁ may be sequenced from a first end of a nucleic acid fragment whereas the second read R₂ may be sequenced from the second end of the nucleic acid fragment. Therefore, nucleotide base pairs of the first read R₁ and second read R₂ may be aligned consistently (e.g., in opposite orientations) with nucleotide bases of the reference genome. Alignment position information derived from the read pair R₁ and R₂ may include a beginning position in the reference genome that corresponds to an end of a first read (e.g., R₁) and an end position in the reference genome that corresponds to an end of a second read (e.g., R₂). In other words, the beginning position and end position in the reference genome represent the likely location within the reference genome to which the nucleic acid fragment corresponds. An output file having SAM (sequence alignment map) format or BAM (binary) format may be generated and output for further analysis such as methylation state determination.

### Example 3—Ability to Detect Cancer as a Function of cfDNA Fraction

In some embodiments, the method further comprises training a classifier to determine a cancer condition of the subject or a likelihood of the subject obtaining the cancer condition using at least tumor fraction estimation information associated with the plurality of variant calls (e.g., based at least in part on one or more respective called variants for one or more corresponding allelic positions of the subject).

For example, in some embodiments, an untrained classifier is trained on a training set comprising one or more reference pluralities of variant calls, where each reference plurality of variant calls is associated with corresponding tumor fraction estimation information.

In some embodiments, the classifier is logistic regression. In some embodiments, the classifier is a neural network algorithm, a support vector machine algorithm, a Naive Bayes algorithm, a nearest neighbor algorithm, a boosted trees algorithm, a random forest algorithm, a decision tree algorithm, a multinomial logistic regression algorithm, a linear model, or a linear regression algorithm.

Classifiers for use in some embodiments are described in further detail in, e.g., U.S. patent application Ser. No. 17/119,606,” filed Dec. 11, 2020, and United States Patent Publication No. 2020-0385813 A1, entitled “Systems and Methods for Estimating Cell Source Fractions Using Methylation Information,” filed Dec. 18, 2019, each of which is hereby incorporated herein by reference in its entirety.

In some embodiments, the classifier is based on a neural network algorithm, a support vector machine algorithm, a decision tree algorithm, an unsupervised clustering algorithm, a supervised clustering algorithm, or a logistic regression algorithm, a mixture model, or a hidden Markov model. In some embodiments, the trained classifier is a multinomial classifier.

In some embodiments the classifier makes use of the B score classifier described in United States Patent Publication Number US 2019-0287649 A1, entitled “Method and System for Selecting, Managing, and Analyzing Data of High Dimensionality,” filed Mar. 13, 2019, which is hereby incorporated by reference.

In some embodiments, the classifier makes use of the M score classifier described in United States Patent Publication No. US 2019-0287652 A1, entitled “Methylation Fragment Anomaly Detection,” filed Mar. 13, 2019, which is hereby incorporated by reference.

In some embodiments, the classifier is a neural network or a convolutional neural network. See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. See also, U.S. Patent Application No. 62/679,746, entitled “Convolutional Neural Network Systems and Methods for Data Classification,” filed Jun. 1, 2018, which is hereby incorporated by reference, for its disclosure of convolutional neural networks that can be used for classifying methylation patterns in accordance with the present disclosure.

In some embodiments, the classifier is a support vector machine (SVM). SVMs are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space corresponds to a non-linear decision boundary in the input space.

In some embodiments, the classifier is a decision tree. Decision trees are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

In some embodiments, the classifier is an unsupervised clustering model. In some embodiments, the classifier is a supervised clustering model. Clustering is described at pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (e.g., similarity measure) is used to ensure that the samples in one cluster are more like one another than they are to samples in other clusters. Second, a mechanism for partitioning the data into clusters using the similarity measure is determined. Similarity measures are discussed in Section 6.7 of Duda 1973, where it is stated that one way to begin a clustering investigation is to define a distance function and to compute the matrix of distances between all pairs of samples in the training set. If distance is a good measure of similarity, then the distance between reference entities in the same cluster will be significantly less than the distance between the reference entities in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” An example of a nonmetric similarity function s(x, x′) is provided on page 218 of Duda 1973. Once a method for measuring “similarity” or “dissimilarity” between points in a dataset has been selected, clustering requires a criterion function that measures the clustering quality of any partition of the data. Partitions of the data set that extremize the criterion function are used to cluster the data. See page 217 of Duda 1973. Criterion functions are discussed in Section 6.8 of Duda 1973. More recently, Duda et al., Pattern Classification, 2^{nd} edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J., each of which is hereby incorporated by reference. Particular exemplary clustering techniques that can be used in the present disclosure include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering (e.g., with no preconceived number of clusters and/or no predetermination of cluster assignments).

In some embodiments, the classifier is a regression model, such as the multi-category logit models described in Agresti, An Introduction to Categorical Data Analysis, 1996, John Wiley & Sons, Inc., New York, Chapter 8, which is hereby incorporated by reference in its entirety. In some embodiments, the classifier makes use of a regression model disclosed in Hastie et al., 2001, The Elements of Statistical Learning, Springer-Verlag, New York.

In some embodiments, the classifier is a Naive Bayes algorithm, such as the tool developed by Rosen et al. to deal with metagenomic reads (See, Bioinformatics 27(1):127-129, 2011). In some embodiments, the classifier is a nearest neighbor algorithm, such as the non-parametric methods described by Kamvar et al., Front Genetics 6:208 doi: 10.3389/fgene.2015.00208, 2015). In some embodiments, the classifier is a mixture model, such as that described in McLachlan et al., Bioinformatics 18(3):413-422, 2002. In some embodiments, in particular, those embodiments including a temporal component, the classifier is a hidden Markov model such as described by Schliep et al., 2003, Bioinformatics 19(1):i255-i263.

In some embodiments, the classifier is an A score classifier. The A score classifier is a classifier of tumor mutational burden based on targeted sequencing analysis of nonsynonymous mutations. For example, a classification score (e.g., “A score”) can be computed using logistic regression on tumor mutational burden data, where an estimate of tumor mutational burden for each individual is obtained from the targeted cfDNA assay. In some embodiments, a tumor mutational burden can be estimated as the total number of variants per individual that are: called as candidate variants in the cfDNA, passed noise-modeling and joint-calling, and/or found as nonsynonymous in any gene annotation overlapping the variants. The tumor mutational burden numbers of a training set can be fed into a penalized logistic regression classifier to determine cutoffs at which 95% specificity is achieved using cross-validation. Additional details on A score can be found, for example, in R. Chaudhary et al., 2017, “Journal of Clinical Oncology, 35(5), suppl.e14529, pre-print online publication, which is hereby incorporated by reference herein in its entirety.

In some embodiments, the classifier is an B score classifier. The B score classifier is described in United States Patent Publication Number US 2019-0287649 A1, entitled “Method and System for Selecting, Managing, and Analyzing Data of High Dimensionality,” which is hereby incorporated by reference. In accordance with the B score method, a first set of sequence reads of nucleic acid samples from healthy subjects in a reference group of healthy subjects are analyzed for regions of low variability. Accordingly, each sequence read in the first set of sequence reads of nucleic acid samples from each healthy subject is aligned to a region in the reference genome. From this, a training set of sequence reads from sequence reads of nucleic acid samples from subjects in a training group is selected. Each sequence read in the training set aligns to a region in the regions of low variability in the reference genome identified from the reference set. The training set includes sequence reads of nucleic acid samples from healthy subjects as well as sequence reads of nucleic acid samples from diseased subjects who are known to have the cancer. The nucleic acid samples from the training group are of a type that is the same as or similar to that of the nucleic acid samples from the reference group of healthy subjects. From this it is determined, using quantities derived from sequence reads of the training set, one or more parameters that reflect differences between sequence reads of nucleic acid samples from the healthy subjects and sequence reads of nucleic acid samples from the diseased subjects within the training group. Then, a test set of sequence reads associated with nucleic acid samples comprising cfNA fragments from a test subject whose status with respect to the cancer is unknown is received, and the likelihood of the test subject having the cancer is determined based on the one or more parameters.

In some embodiments, the classifier is an M score classifier. The M score classifier is described in United States Patent Publication No. US 2019-0287652 A1, entitled “Anomalous Fragment Detection and Classification,” which is hereby incorporated by reference.

### Example 4—Whole Genome Bisulfite Sequencing (WGBS)

WGBS is described in United States Patent Application Publication No. US 2019-0287652 A1 entitled “Anomalous Fragment Detection and Classification,” which is hereby incorporated by reference.

### Example 5—Cell-Free Genome Atlas Study (CCGA) Cohorts

Subjects from the CCGA [NCT02889978] were used in the Examples of the present disclosure. CCGA is a prospective, multi-center, observational cfDNA-based early cancer detection study that has enrolled 15,254 demographically-balanced participants at 141 sites. Blood samples were collected from the 15,254 enrolled participants (56% cancer, 44% non-cancer) from subjects with newly diagnosed therapy-naive cancer (C, case) and participants without a diagnosis of cancer (noncancer [NC], control) as defined at enrollment. \

In a first cohort (pre-specified substudy) (CCGA-1), plasma cfDNA extractions were obtained from 3,583 CCGA and STRIVE participants (CCGA: 1,530 cancer subjects and 884 non-cancer subjects; STRIVE 1,169 non-cancer participants). STRIVE is a multi-center, prospective, cohort study enrolling women undergoing screening mammography (99,259 participants enrolled). Blood was collected (n=1,785) from 984 CCGA participants with newly diagnosed, untreated cancer (20 tumor types, all stages) and 749 participants with no cancer diagnosis (controls) for plasma cfDNA extraction. This preplanned substudy included 878 cases, 580 controls, and 169 assay controls (n=1627) across twenty tumor types and all clinical stages.

Three sequencing assays were performed on the blood drawn from each participant: 1) paired cfDNA and white blood cell (WBC)-targeted sequencing (60,000×, 507 gene panel) for single nucleotide variants/indels (the ART sequencing assay); a joint caller removed WBC-derived somatic variants and residual technical noise; 2) paired cfDNA and WBC whole-genome sequencing (WGS; 35×) for copy number variation; a novel machine learning algorithm generated cancer-related signal scores; joint analysis identified shared events; and 3) cfDNA whole-genome bisulfite sequencing (WGBS; 34×) for methylation; normalized scores were generated using abnormally methylated fragments. In addition, tissue samples were obtained from participants with cancer only, such that 4) whole-genome sequencing (WGS; 30×) was performed on paired tumor and WBC gDNA for identification of tumor variants for comparison.

Within the context of the CCGA-1 study, several methods were developed for estimating tumor fraction of a cfDNA sample. See, International Patent Publication No. WO/2019/204360, entitled “SYSTEMS AND METHODS FOR DETERMINING TUMOR FRACTION IN CELL-FREE NUCLEIC ACID,” International Patent Publication No. WO 2020/132148, entitled “SYSTEMS AND METHODS FOR ESTIMATING CELL SOURCE FRACTIONS USING METHYLATION INFORMATION,” and United States Patent Publication Number US 2020-0340064 A1, entitled “SYSTEMS AND METHODS FOR TUMOR FRACTION ESTIMATION FROM SMALL VARIANTS,” each of which is hereby incorporated by reference.

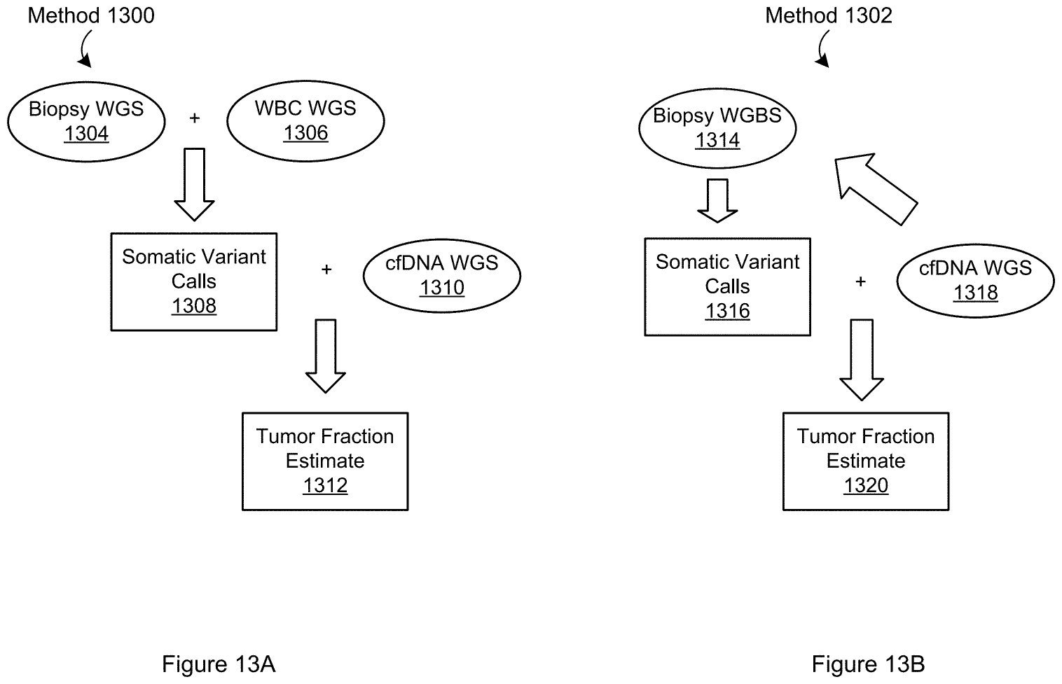

For example, one of the approaches was illustrated as method 1300 in FIG. 13A. In this approach, nucleic acid samples from formalin-fixed, paraffin-embedded (FFPE) tumor tissues (e.g., 1304) and nucleic acid samples from white blood cells (WBC) from the matching patient (e.g., 1306) were sequenced by whole-genome sequencing (WGS). Somatic variants identified based on the sequencing data (e.g., 1308) were analyzed against matching cfDNA sequencing data from the same patient (e.g., 1310) were used to determine a tumor fraction estimate (e.g., 1312).

In particular, method 1300 in FIG. 13A requires the use of whole genome sequencing of a biopsy 1304 and matched white blood cell whole genome sequencing 1306 to determine a set of potentially informative somatic variant calls (e.g., 1308). Germline variants are typically not involved with the development of cancer and as such typically provide less information than somatic variants in terms of detecting and/or identifying cancer. Method 1300, in some embodiments, continues by obtaining 1310 whole genome sequencing information of cell-free DNA of a test subject. The combination of known somatic variant calls 1308 as the search space and subject-specific variants 1310 then can be used to provide a tumor fraction estimate 1312 for the subject.

Method 1302 in FIG. 13B, in contrast, does not incorporate information from white blood cell sequencing. Instead, method 1302 uses information from biopsy whole genome bisulfite sequencing 1314 to generate a set of somatic variant calls 1316. In some embodiments, the set of somatic variants differs 1316 from the set of somatic variants 1308 determined in method 1300. Method 1302, in some embodiments, proceeds by obtaining whole genome sequencing of cell-free DNA 1318 for a test subject. The combination of somatic variant calls 1316 as the search space and subject-specific variants from the cell-free DNA sequencing 1318 can then be used to provide a tumor fraction estimate 1312 for the subject. In some embodiments, for methods 1300 and 1302, blocks 1304, 1306, and 1314 are performed for a set of reference subjects. In some embodiments of methods 1300 and 1302, one or more of the blocks 1304, 1306, or 1314 are performed on the respective test subject.

FIG. 14 provides an example process for the method outlined in FIG. 13B, while FIG. 15 illustrates an example of filtering variants in order to improve the positive predictive value (PPV) of variant calls in accordance with the method of FIG. 13B.

In a second pre-specified substudy (CCGA-2), a targeted, rather than whole-genome, bisulfite sequencing assay was used to develop a classifier of cancer versus non-cancer and tissue-of-origin based on a targeted methylation sequencing approach. For CCGA-2, 3,133 training participants and 1,354 validation samples (775 having cancer; 579 not having cancer as determined at enrollment, prior to confirmation of cancer versus non-cancer status) were used. Plasma cfDNA was subjected to a bisulfite sequencing assay (the COMPASS assay) targeting the most informative regions of the methylome, as identified from a unique methylation database and prior prototype whole-genome and targeted sequencing assays, to identify cancer and tissue-defining methylation signal. Of the original 3,133 samples reserved for training, only 1,308 samples were deemed clinically evaluable and analyzable. Analysis was performed on a primary analysis population n=927 (654 cancer and 273 non-cancer) and a secondary analysis population n=1,027 (659 cancer and 373 non-cancer). Finally, genomic DNA from formalin-fixed, paraffin-embedded (FFPE) tumor tissues and isolated cells from tumors was subjected to whole-genome bisulfite sequencing (WGBS) to generate a large database of cancer-defining methylation signals for use in panel design and in training to optimize performance.

These data demonstrate the feasibility of achieving >99% specificity for invasive cancer, and support the promise of cfDNA assay for early cancer detection. See, e.g., Klein et al., 2018, “Development of a comprehensive cell-free DNA (cfDNA) assay for early detection of multiple tumor types: The Circulating Cell-free Genome Atlas (CCGA) study,” J. Clin. Oncology 36(15), 12021-12021; doi: 10.1200/JCO.2018.36.15_suppl.12021, and Liu et al., 2019, “Genome-wide cell-free DNA (cfDNA) methylation signatures and effect on tissue of origin (TOO) performance,” J. Clin. Oncology 37(15), 3049-3049; doi: 10.1200/JCO.2019.37.15 suppl.3049, each of which is hereby incorporated herein by reference in its entirety.

Within the context of the CCGA-2 study, multiple methods were developed for estimating tumor fraction of a cfDNA sample based on methylation data (obtained by targeted methylation or WGBS) (see e.g., International Patent Publication No. WO 2020/132148, entitled “SYSTEMS AND METHODS FOR ESTIMATING CELL SOURCE FRACTIONS USING METHYLATION INFORMATION,” and U.S. Provisional Pat. Appl. No. 62/983,443 entitled “Identifying Methylation Patterns that Discriminate or Indicate a Cancer Condition,” filed Feb. 28, 2020, each of which is hereby incorporated by reference in its entirety). For example, one of the approaches was illustrated as method 1302 in FIG. 13B. In this approach, nucleic acid samples from formalin-fixed, paraffin-embedded (FFPE) tumor tissues (e.g., 1314) were analyzed by whole-genome bisulfite sequencing (WGBS). Somatic variants identified based on the sequencing data (e.g., 1316) were analyzed against matching cfDNA WGBS sequencing data from the same patient (e.g., 1318) were used to determine a tumor fraction estimate (e.g., 1320). An example of tumor fraction analysis based on WGBS sequencing data can be found in Example 7.

### Example 6—Generation of a Methylation State Vector in Accordance with Some Embodiments of the Present Disclosure

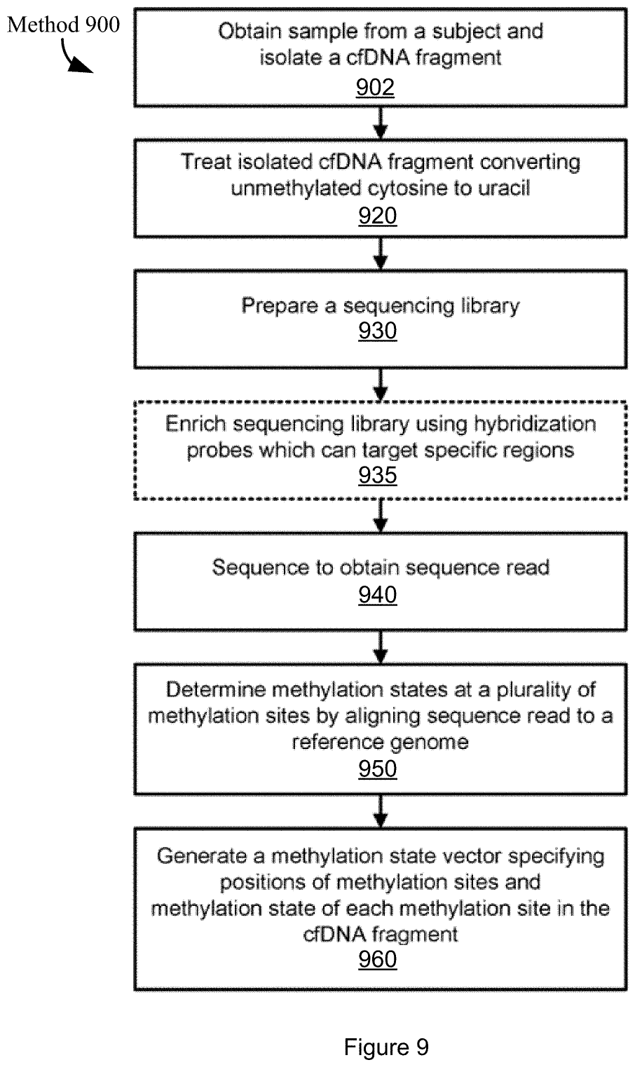

FIG. 9 is a flowchart describing a process 900 of sequencing a fragment of cfDNA to obtain a methylation state vector, according to an embodiment in accordance with the present disclosure.

Referring to block 902, the cfDNA fragments are obtained from the biological sample (e.g., as discussed above in conjunction with FIGS. 3A-3D). Referring to block 920, the cfDNA fragments are treated to convert unmethylated cytosines to uracils. In some embodiments, the cfDNA is subjected to a bisulfite treatment that converts the unmethylated cytosines of the fragment of cfDNA to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, Calif.)) is used for the bisulfite conversion in some embodiments. In other embodiments, the conversion of unmethylated cytosines to uracils is accomplished using an enzymatic reaction. For example, the conversion can use a commercially available kit for converting unmethylated cytosines to uracils, such as APOBEC-Seq (NEBiolabs, Ipswich, Mass.).

From the converted cfDNA fragments, a sequencing library is prepared (block 930). Optionally, the sequencing library is enriched 935 for cfDNA fragments, or genomic regions, that are informative for cancer status using a plurality of hybridization probes. The hybridization probes are short oligonucleotides capable of hybridizing to particularly specified cfDNA fragments, or targeted regions, and enriching for those fragments or regions for subsequent sequencing and analysis. Hybridization probes may be used to perform targeted, high-depth analysis of a set of specified CpG sites of interest to the researcher. Once prepared, the sequencing library or a portion thereof can be sequenced to obtain a plurality of sequence reads (940). The sequence reads may be in a computer-readable, digital format for processing and interpretation by computer software.

From the sequence reads, a location and methylation state for each of CpG site is determined based on the alignment of the sequence reads to a reference genome (950). A methylation state vector for each fragment specifying a location of the fragment in the reference genome (e.g., as specified by the position of the first CpG site in each fragment, or another similar metric), a number of CpG sites in the fragment, and the methylation state of each CpG site in the fragment (960).

### Example 7—Tumor Fraction Estimation Based on Detection of Somatic Variants

Tumor fraction was estimated from the observed counts of fragments with tumor features in cfDNA. Genetic small nucleotide variant and methylation variant tumor features were determined from WGBS of tumor tissue biopsies. A subset of 231 participants had matched tumor biopsy and cfDNA sequencing in the training set and were used in the tumor fraction estimations. This set of participants excluded those whose biopsies were used in target selection.

More specifically, to calculate the tumor-fraction from SNVs, a joint analysis of WGBS of tumor tissue and WGS of cfDNA was performed to identify tumor-associated somatic small nucleotide variants, for example, as illustrated in method 1302 in FIG. 13B. Method 1302 of FIG. 13B includes calling SNVs within WGBS tissue using the variant caller detailed above in conjunction with FIG. 3 that accounted for the effects of bisulfite conversion (unmethylated C-to-T conversion) by using strand-specific pileups and a Bayesian genotype model. Additional elements of method 1302 are provided in FIG. 14B (e.g., blocks 1402-1420).

Specifically, method 1302 comprises calling WGBS tissue somatic variant calls 1402/1404 using WGBS tissue sequencing data 1402 (and the methods disclosed in FIGS. 3B through 3D) and WGS cfDNA sequencing data 1418. WGS cfDNA data 1418 is analyzed (e.g., using the freebayes package) to determine a plurality of germline variant calls 1420. Meanwhile, WGBS tissue sequencing data 1402 is used as the baseline from which various uninformative sets of variants are removed (e.g., blocks 1404-1416), resulting in a set of somatic variant calls.

In accord with block 1404 of FIG. 14, each variant allele that is identified using the systems and methods described in conjunction with FIGS. 3B through 3D (block 1404) as a candidate WGBS variant (block 1406), in order to be retained must not be identified as a germline variant (block 1408).

In accord with block 1408 of FIG. 14, in some embodiments, a candidate variant allele from block 1406 is identified as a germline variant and removed from the list of candidate variants when a variant caller algorithm, such as FreeBayes, VarDict, MuTect, MuTect2, MuSE, FreeBayes, VarDict, and/or MuTect (see Bian, 2018, “Comparing the performance of selected variant callers using synthetic data and genome segmentation,” BMC Bioinformatics 19:429, which is hereby incorporated by reference) identifies the variant as a germline variant, private to a test subject within sample-matched WGS cfDNA (blocks 1418 and 1420).

In accord with block 1410 of FIG. 14, in addition to removal of germline variants private to the test subject 14A (block 1408), variants that are known germline variants in public databases such as the gnomAD and dbDNP datasets are also removed from the list of candidate WGBS variants. For information on such datasets, see Karczewski et al., 2019, “Variation across 141,456 human exomes and genomes reveals the spectrum of loss-of-function intolerance across human protein-coding genes,” bioRxiv doi.org/10.1101/531210 and Sherry et al., 2011, “dbSNP: the NCBI database of genetic variation” Nuc. Acids. Res. 29, 308-311.

In accord with block 1412 of FIG. 14, in addition to the removal of germline variants private to the test subject (block 1408), as well as variants that are known germline variants in public databases such as the gnomAD and dbDNP datasets (block 1410) the list of candidate WGBS variants (block 1406), candidate WGBS variants that appear at least twice in the CCGA I dataset of 642 subjects are also removed from the list of WGBS variants. In some embodiments, rather than using a threshold of 2, a threshold of 3, 4, 5, 6, 7, 8, 9 or 10 is used, meaning that the variant must appear in 3, 4, 5, 6, 7, 8, 9 or 10 more subjects in the cohort (e.g., the CCGA I dataset of 642 subjects) to be eliminated in block 1412.

In accord with block 1414 of FIG. 14, in addition to the removal of germline variants private to the test subject (block 1408), variants that are known germline variants in public databases such as the gnomAD and dbDNP datasets (block 1410), respective variants that appear at least twice in a reference cohort (block 1412), variants that appear with less than a minimum frequency across the unique test fragments of the test subject mapping to such variants (minimum variant allele frequency) or greater than a maximum frequency (maximum variant allele frequency) across the unique test fragments of the test subject mapping to such variants are removed from the list of candidate WGBS variant allele fragments. For instance, in some embodiments a respective variant allele must occur in at least 20% of the nucleic acid fragments from the test subject mapping to the respective allele position for the variant allele to be retained in block 1414. In alternative embodiments, the minimum allele frequency is at least 3%, at least 5%, at least 10%, at least 15%, at least 25%, at least 30%, at least 35%, at least 40%, at least 45%, or at least 50% of the nucleic acid fragments from the test subject. Moreover, in some embodiments, each candidate variant allele must have a maximum variant allele frequency (maximum VAF) of 90% in order to be retained in block 1414. That is, the variant allele must occur in no more than 90% of the nucleic acid fragments from the test subject. In alternative embodiments, the maximum allele frequency 95% or less, 85% or less, 80% or less, 75% or less, 70% or less, 65% or less, 60% or less, 55% or less, or 50% or less of the nucleic acid fragments from the test subject. Further still, in order to be retained for further use in a pipeline, in some embodiments the variant allele must be supported by an overall sequencing depth of at least 10 in order to not be eliminated in block 1414. In other words, the sequence reads from the test subject must include sequencing information for at least 10 different nucleic acid fragments from the test subject that map to the genomic region of the variant allele. This depth requirement does not impose a requirement that each of these nucleic acid fragments have the variant allele. In alternative embodiments, the sequence reads from the test subject must include sequencing information for at least 15, 20, 25, 30, 35, 40, 45, 50, 55, 60, 65, 70, 75, 80, 85, 90, 95, 100, 200, 300, 400, 500, or 1000 nucleic acid fragments from the test subject that map to the genomic region of the variant allele in order for the variant allele to not be eliminated from the candidate WGBS variants in block 1414.

With respect to FIG. 15, in accord with method 1302 of FIG. 13B and FIG. 14, once a candidate list of 44 SNVs is generated (e.g., the tissue minimum alternate allele 1432), analysis of the performance of tumor fraction estimation after each of the filtering stages detailed above in FIG. 14 (e.g., 1434-1446) was analyzed. These performance statistics show that the filtering stages enrich for somatic variants even though a matched-normal reference for these individuals was not available. These filters included a minimum variant allele frequency 1434 (e.g., a minimum VAF of 20%) of block 1416 of FIG. 14, and maximum variant allele frequency 1436 (e.g., a maximum VAF of 90%) of block 1416 of FIG. 14, a minimum depth 1438 (e.g., a depth of 10) of block 1416 of FIG. 14, a custom blacklist of known noisy sites 1444 (which is, in some embodiments, based on a set of 642 samples from the CCGA Approach 1 method described above in Example 5) of block 1412 of FIG. 14, the removal of germline-variants private to a test subject as marked by freebayes within sample-matched WGS cfDNA 1446 of block 1408 of FIG. 14, and the removal (e.g., blacklisting) of generally known germline variants using the dbSNP and gnomAD datasets (see e.g., 1440 and 1442, respectively) of block 1410 of FIG. 14. In some embodiments, these filters are applied to a dataset in any ordering.

Counts of fragments supporting and not supporting each variant were generated from WGS sequencing of corresponding cfDNA samples matched to the WGBS data. Posterior tumor fraction estimates were calculated using a grid search over tumor fractions and employing a per-variant likelihood defined as a mixture of binomial likelihoods. The mixture components accounted for (1) observing fragments due to tumor shedding as well as (2) various error modes including germline variants and falsely called variants. Median and 95% credible intervals were calculated for each participant's tumor fraction.

The resulting combination (e.g., 1448—the homozygous reference likelihood) of the above-described filters results in improved performance over the use of any one or any other combination of a subset of the individual filters (e.g., 1434-1446). For example, the filter 1448 has a resulting sensitivity of 32.2% and positive predictive value of 49.5%. In contrast, the tissue minimum alternate allele set 1432 provides a high sensitivity (e.g., 68.72%); however, there is a concurrent low positive predictive value of only 0.02%. The sensitivity (sens) and positive predictive value (PPV) of each other filter is indicated in FIG. 15. The positive predictive value (PPV) refers to the proportion of variants that are correctly categorized as associated with cancer (e.g., the number of true positives divided by the sum of the number of true positives and the number of false positives).

### CONCLUSION

The terminology used herein is for the purpose of describing particular cases only and is not intended to be limiting. As used herein, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

As used herein, the term “if” may be construed to mean “when” or “upon” or “in response to determining” or “in response to detecting,” depending on the context. Similarly,

1. A method of calling a variant at an allelic position in a test subject, the method comprising:
at a computer system having one or more processors, and memory storing one or more programs for execution by the one or more processors:
(A) deriving a prior probability of genotype at the allelic position, for each respective candidate genotype in a set of candidate genotypes, using nucleic acid data acquired from a reference population;
(B) obtaining, for the allelic position, a strand-specific base count set, wherein the strand-specific base count set comprises a strand-specific count for each base in the set of bases {A, C, T, G} at the allelic position, in a forward direction and a reverse direction, that is acquired by determining (i) a strand orientation and (ii) an identity of a respective base at the allelic position in each respective nucleic acid fragment sequence in a first plurality of nucleic acid fragment sequences, in electronic form, that map to the allelic position, acquired from a first plurality of nucleic acid fragments in a first biological sample of the test subject by a methylation sequencing, wherein the first plurality of nucleic acid fragment sequences is 5 or more nucleic acid fragment sequences, wherein the obtaining comprises determining the first plurality of nucleic acid fragments from 10,000 or more sequence reads from the first biological sample of the test subject by the methylation sequencing, and wherein bases at the allelic position in the first plurality of nucleic acid fragment sequences whose identity can be affected by conversion of methylated or unmethylated cytosine do not contribute to the strand-specific base count set;
(C) computing a respective forward strand conditional probability and a respective reverse strand conditional probability for each respective candidate genotype in the set of candidate genotypes for the allelic position using the strand-specific base count set and a sequencing error estimate thereby computing a plurality of forward strand conditional probabilities and a plurality of reverse strand conditional probabilities;
(D) computing a plurality of likelihoods, each respective likelihood in the plurality of likelihoods for a respective candidate genotype in the set of candidate genotypes, using a combination of (i) the respective forward strand conditional probability for the respective candidate genotype in the plurality of forward strand conditional probabilities, (ii) the respective reverse strand conditional probability for the respective candidate genotype in the plurality of reverse strand conditional probabilities, and (iii) the prior probability of genotype for the respective candidate genotype; and
(E) determining whether the plurality of likelihoods support a variant call at the allelic position.

2. The method of claim 1, wherein the first biological sample is a liquid biological sample and each respective nucleic acid fragment sequence in the first plurality of nucleic acid fragment sequences represents all or a portion of a respective cell-free nucleic acid molecule in a population of cell-free nucleic acid molecules in the liquid biological sample.

3-4. (canceled)

5. The method of claim 1, wherein the reference population comprises at least one hundred reference subjects.

6. The method of claim 1, wherein the first biological sample comprises or consists of blood, whole blood, plasma, serum, urine, cerebrospinal fluid, fecal, saliva, sweat, tears, pleural fluid, pericardial fluid, or peritoneal fluid of the test subject.

7-8. (canceled)

9. The method of claim 1, wherein the forward direction is a F1R2 read orientation and the reverse direction is a F2R1 read orientation.

10. The method of claim 1, wherein each respective candidate genotype in the set of genotypes is of the form X/Y, wherein:
X is an identity of the base in the set of bases set of bases {A, C, T, G} at the allelic position in a reference genome,
Y is an identity of the base in the set of bases set of bases {A, C, T, G} at the allelic position in the test subject.

11. The method of claim 10, wherein the set of candidate genotypes consists of between two and ten genotypes in the set {A/A, A/C, A/G, A/T, C/C, C/G, C/T, G/G, G/T, and T/T}.

12-34. (canceled)

35. The method of claim 1, wherein the methylation sequencing is whole genome methylation sequencing.

36. The method of claim 1, wherein the methylation sequencing is targeted DNA methylation sequencing using one hundred or more probes.

37. (canceled)

38. The method of claim 1, wherein the methylation sequencing detects one or more 5-methylcytosine (5mC) and/or 5-hydroxymethylcytosine (5hmC) in respective nucleic acid fragments in the first plurality of nucleic acid fragments.

39-41. (canceled)

42. The method of claim 1, wherein the methylation sequencing is bisulfite sequencing.

43. The method of claim 1, wherein the allelic position is a single base position and the variant is a single nucleotide polymorphism.

44. The method of claim 1, wherein the sequencing error estimate is 0.01 to 0.0001.

45. The method of claim 10, wherein the determining whether the plurality of likelihoods support a variant call at the allelic position comprises:
determining whether the likelihood in the plurality of likelihood corresponding to the reference genotype for the allelic position satisfies a variant threshold, wherein when the allelic position satisfies a variant threshold, a variant at the allelic position is called.

46. The method of claim 45, wherein the likelihood is expressed as a log-likelihood and the variant threshold is satisfied when the log-likelihood for the reference genotype for the allelic position is less than −10.

47. The method of claim 45, wherein the likelihood is expressed as a log-likelihood and the variant threshold is between −25 and −5.

48. The method of claim 45, wherein the method further comprises, when a variant at the allelic position is called, determining an identity of the variant by selecting the candidate genotype in the set of candidate genotypes for the allelic position that has the best likelihood in the plurality of likelihoods as the variant.

49. The method of claim 45, wherein the reference genotype for the allelic position is A/A, G/G, C/C or T/T.

50. The method of claim 1, the method further comprising performing the (A) obtaining, (B) obtaining, (C) computing, (D) computing, and (E) determining for each allelic position in a plurality of allelic positions thereby obtaining a plurality of variant calls for the test subject, wherein each variant call in the plurality of variant calls is at a different genomic position in a reference genome.

51. The method of claim 1, the method further comprising performing the (A) obtaining, (B) obtaining, (C) computing, (D) computing, and (E) determining for each allelic position in a plurality of allelic positions thereby obtaining a plurality of variant calls for the test subject, wherein each variant call in the plurality of variant calls is at a different genomic position in a reference genome, and wherein
the plurality of variant calls comprises 200 variant calls,
the first biological sample is a tissue sample, and
the methylation sequencing is whole genome bisulfite sequencing.

52. (canceled)

53. The method of claim 51, the method further comprising:
obtaining a second plurality of variant calls using a second plurality of nucleic acid fragment sequences, in electronic form, acquired from a second plurality of nucleic acid fragments in a second biological sample of the test subject by whole genome sequencing, wherein the second plurality of nucleic acid fragments are cell-free nucleic acid fragments and wherein the second biological sample is a liquid biological sample; and
removing a respective variant call from the plurality of variant calls that is also in the second plurality of variant calls.

54. The method of claim 51, the method further comprising:
removing a respective variant call from the plurality of variant calls that is in a list of known germline variants,
removing a respective variant call from the plurality of variant calls when the respective variant call is found in a tissue sample of a subject other than the test subject,
removing a respective variant call from the plurality of variant calls when the respective variant call fails to satisfy a quality metric.

55-56. (canceled)

57. The method of claim 54, wherein the quality metric is a minimum variant allele fraction in the first plurality of nucleic acid fragment sequences, in electronic form, that map to the allelic position of the respective variant call.

58. The method of claim 57, wherein the minimum variant allele fraction is ten percent.

59. The method of claim 56, wherein:
the quality metric is a maximum variant allele fraction in the first plurality of nucleic acid fragment sequences, in electronic form, that map to the allelic position of the respective variant call, or
the quality metric is a minimum depth in the first plurality of nucleic acid fragment sequences, in electronic form, that map to the allelic position of the respective variant call.

60-62. (canceled)

63. The method of claim 53, the method further comprising using the plurality of variant calls, after the removing, to perform tumor fraction estimation, to quantify white blood cell clonal expansion, or to assess a genetic risk of the subject through germline analysis using the plurality of variant calls.

64-65. (canceled)

66. The method of claim 50, wherein the determining (E) step further comprises filtering the plurality of variant calls by one or more filters, wherein the one or more filters are selected from the group consisting of a minimum variant allele frequency, a maximum variant allele frequency, a minimum depth, blacklisting germline variants from the test subject, and blacklisting germline variants from a reference database.

67. (canceled)

68. A computing system, comprising:
one or more processors;
memory storing one or more programs to be executed by the one or more processor, the one or more programs comprising instructions for calling a variant at an allelic position in a test subject by a method comprising:
A) obtaining a prior probability of genotype at the allelic position, for each respective candidate genotype in a set of candidate genotypes, using nucleic acid data acquired from a reference population;
(B) obtaining, for the allelic position, a strand-specific base count set, wherein the strand-specific base count set comprises a strand-specific count for each base in the set of bases {A, C, T, G} at the allelic position, in a forward direction and a reverse direction, that is acquired by determining (i) a strand orientation and (ii) an identity of a respective base at the allelic position in each respective nucleic acid fragment sequence in a first plurality of nucleic acid fragment sequences, in electronic form, that map to the allelic position, acquired from a first plurality of nucleic acid fragments in a first biological sample of the test subject by a methylation sequencing, wherein the first plurality of nucleic acid fragment sequences is 5 or more nucleic acid fragment sequences, the first plurality of nucleic acid fragments is collectively determined from 10,000 or more sequence reads obtained from the first biological sample of the test subject by the methylation sequencing, and wherein bases at the allelic position in the first plurality of nucleic acid fragment sequences whose identity can be affected by conversion of unmethylated cytosine to uracil do not contribute to the strand-specific base count set;
(C) computing a respective forward strand conditional probability and a respective reverse strand conditional probability for each respective candidate genotype in the set of candidate genotypes for the allelic position using the strand-specific base count set and a sequencing error estimate thereby computing a plurality of forward strand conditional probabilities and a plurality of reverse strand conditional probabilities;
(D) computing a plurality of likelihoods, each respective likelihood in the plurality of likelihoods for a respective candidate genotype in the set of candidate genotypes, using a combination of (i) the respective forward strand conditional probability for the respective candidate genotype in the plurality of forward strand conditional probabilities, (ii) the respective reverse strand conditional probability for the respective candidate genotype in the plurality of reverse strand conditional probabilities, and (iii) the prior probability of genotype for the respective candidate genotype; and
(E) determining whether the plurality of likelihoods support a variant call at the allelic position.

69. A non-transitory computer readable storage medium storing one or more programs for calling a variant at an allelic position in a test subject, the one or more programs configured for execution by a computer, wherein the one or more programs comprise instructions for:
A) obtaining a prior probability of genotype at the allelic position, for each respective candidate genotype in a set of candidate genotypes, using nucleic acid data acquired from a reference population;
(B) obtaining, for the allelic position, a strand-specific base count set, wherein the strand-specific base count set comprises a strand-specific count for each base in the set of bases {A, C, T, G} at the allelic position, in a forward direction and a reverse direction, that is acquired by determining (i) a strand orientation and (ii) an identity of a respective base at the allelic position in each respective nucleic acid fragment sequence in a first plurality of nucleic acid fragment sequences, in electronic form, that map to the allelic position, acquired from a first plurality of nucleic acid fragments in a first biological sample of the test subject by a methylation sequencing, wherein the first plurality of nucleic acid fragment sequences is 5 or more nucleic acid fragment sequences, the first plurality of nucleic acid fragments is collectively determined from 10,000 or more sequence reads obtained from the first biological sample of the test subject by the methylation sequencing, and wherein bases at the allelic position in the first plurality of nucleic acid fragment sequences whose identity can be affected by conversion of unmethylated cytosine to uracil do not contribute to the strand-specific base count set;
(C) computing a respective forward strand conditional probability and a respective reverse strand conditional probability for each respective candidate genotype in the set of candidate genotypes for the allelic position using the strand-specific base count set and a sequencing error estimate thereby computing a plurality of forward strand conditional probabilities and a plurality of reverse strand conditional probabilities;
(D) computing a plurality of likelihoods, each respective likelihood in the plurality of likelihoods for a respective candidate genotype in the set of candidate genotypes, using a combination of (i) the respective forward strand conditional probability for the respective candidate genotype in the plurality of forward strand conditional probabilities, (ii) the respective reverse strand conditional probability for the respective candidate genotype in the plurality of reverse strand conditional probabilities, and (iii) the prior probability of genotype for the respective candidate genotype; and
(E) determining whether the plurality of likelihoods support a variant call at the allelic position.
